(19) United States
(12) Patent Application Publication
(10) Pub. No.: US 2010/0286112 A1
(43) Pub. Date: Nov. 11, 2010

(54) COMPOUNDS FOR THE TREATMENT OF METABOLIC DISORDERS

(76) Inventors: Oscar Barba, Oxfordshire (GB); Stuart Edward Bradley, Oxfordshire (GB); Matthew Colin Thor Fyfe, Oxfordshire (GB); Patrick Eric Hanrahan, Oxfordshire (GB); Thomas Martin Krulle, Oxfordshire (GB); Martin James Procter, Oxfordshire (GB); Christine Reynet McCormack, Oxfordshire (GB); Karen Lesley Schofield, Oxfordshire (GB); Donald Smyth, Oxfordshire (GB); Alan John William Stewart, Oxfordshire (GB); Simon Andrew Swain, Oxfordshire (GB); Peter Widdowson, Oxfordshire (GB)

Correspondence Address: OSI PHARMACEUTICALS, INC., 420 Saw Mill River Road, Ardsley, NY, 10502, US

(21) Appl. No.: 12/677,153

(22) PCT Filed: Sep. 10, 2008
(86) PCT No.: PCT/GB08/50814

§ 371 Date: Jun. 30, 2010

(30) Foreign Application Priority Data
Sep. 10, 2007 (GB) 0717568.0
Sep. 10, 2007 (GB) 0717569.8
Sep. 10, 2007 (GB) 0717570.6
Sep. 10, 2007 (GB) 0717571.4
Sep. 10, 2007 (GB) 0717572.2
Sep. 10, 2007 (GB) 0717573.0

(51) Int. Cl. A61K 31/454; C07D 417/14; A61P 3/10; A61P 3/04; A61P 3/06; A61P 9/12; C07D 401/04; A61K 31/506; C07D 207/04; A61K 31/40; C07D 277/04; A61K 31/426

(52) U.S. Cl. 514/210.18; 546/209; 514/326; 544/332; 514/275; 548/540; 514/423; 548/200; 514/365

(57) ABSTRACT

The present invention is directed to therapeutic compounds which have dual activity as agonists of GPR119 and inhibitors of DPP-IV and are useful for the treatment of metabolic disorders including type II diabetes.

### BACKGROUND OF THE INVENTION

The present invention is directed to therapeutic compounds useful for the treatment of metabolic disorders including type II diabetes. In particular, the present invention is directed to compounds which have dual activity as agonists of GPR119 and inhibitors of DPP-IV.

Drugs aimed at the pathophysiology associated with non-insulin dependent Type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) places people at high risk of coronary artery disease, and is characterized by a cluster of risk factors including central obesity (excessive fat tissue in the abdominal region), glucose intolerance, high triglycerides and low HDL cholesterol, and high blood pressure. Myocardial ischemia and microvascular disease is an established morbidity associated with untreated or poorly controlled metabolic syndrome.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

There is a continuing need for novel antidiabetic agents, particularly ones that are well tolerated with few adverse effects and in particular for agents which are weight neutral or preferably cause weight loss.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of diabetes.

GPR119 agonists have been shown to stimulate the release of GLP-1 from the GI tract. In doing so, GPR119 agonists (1) enhance glucose-dependent insulin release from the pancreas leading to improvements in oral glucose tolerance; (2) attenuate disease progression by increasing β-cell cAMP concentrations; and (3) induce weight loss possibly through GLP-1's ability to reduce food intake.

International Patent Applications WO2005/061489, WO2006/070208, WO2006/067532, WO2006/067531, WO2007/003960, WO2007/003961, WO2007/003962 and WO2007/003964, WO2007/116229, WO2007/116230, WO2008/081204, WO2008/081205, WO2008/081206, WO2008/081207, WO2008/081208 disclose GPR119 receptor agonists.

Dipeptidyl peptidase IV (DPP-IV) is a ubiquitous, yet highly specific, serine protease that cleaves N-terminal dipeptides from polypeptides with L-proline or L-alanine at the penultimate position. F or the treatment of diabetes, studies with DPP-IV inhibitors show the principle role of DPP-IV in the inactivation GLP-1. By extending the duration of action of GLP-1, insulin secretion is stimulated, glucagon release inhibited, and gastric emptying slowed. Examples of DPP-IV inhibitors include vildagliptin, sitagliptin and saxagliptin.

The possibility of using a combination of a GPR119 agonist and a DPP-IV has been suggested, however this requires the administration of two separately formulated products to the patient or the co-formulation of two active ingredients with the inherent problems of achieving compatability in the physicochemical and pharmacokinetic and pharmacodynamic properties of the two active ingredients.

### SUMMARY OF THE INVENTION

The present invention is directed to compounds which have dual activity as agonists of GPR119 and inhibitors of DPP-IV and are useful for the treatment of metabolic disorders including type II diabetes.

### DETAILED DESCRIPTION OF THE INVENTION

The present invention is directed to a compound which is an agonist of GPR119 and an inhibitor of DPP-IV, or a pharmaceutically acceptable salt thereof.

The compounds of the invention preferably comprise an α-aminoacylpyrrolidine or α-aminoacylthiazolidine group.

Specific compounds of the invention which may be mentioned are compounds of formula (I) and pharmaceutically acceptable salts thereof:

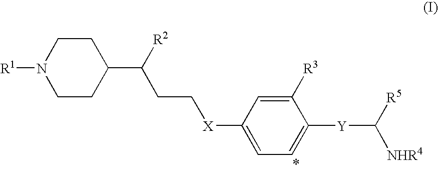

wherein R¹ is —C(O)—O—C₂₋₄ alkyl, or R¹ is:

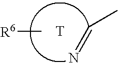

where T together with the —N═C— to which it is attached forms a 5- or 6-membered heteroaryl ring optionally containing up to 2 additional heteroatoms selected from N, O and S;

when T together with the —N═C— to which it is attached forms a 5-membered heteroaryl ring, R⁶ is C₂₋₄ alkyl, and when T together with the —N═C— to which it is attached forms a 6-membered heteroaryl ring, R⁶ is C₂₋₄ alkyl, fluoro or chloro;

R² is hydrogen or methyl;

R³ is hydrogen, fluoro or chloro, or when R⁷ is cyano, R³ may be methyl;

R⁴ is hydrogen or, when Y is —CH₂— or —CHMe-, R⁴ can be —CH₂— linked to position * on the phenyl ring to form a fused 6-membered N-containing heterocycle;

R⁵ is benzyl optionally substituted by one or more fluoro, chloro, cyano or methyl groups, or R⁵ is:

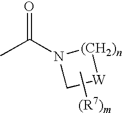

where n is 1 or 2 and m is 0, 1 or 2;

W is CH₂ or, when n is 2, W may be S;

when W is CH₂, R⁷ is fluoro or cyano, and when W is S, R⁷ is cyano;

X is —O— or —CH₂—; and

Y is a bond, —CH₂— or —CHMe-.

In the compounds of formula (I) R¹ is preferably —C(O)—O-isopropyl or —C(O)—O-tert-butyl, or R¹ is:

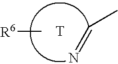

where T together with the —N═C— to which it is attached forms a 5- or 6-membered heteroaryl ring optionally containing up to 2 additional heteroatoms selected from N and O, e.g. oxadiazole or pyrimidine, especially oxadiazole, and R⁶ is as described in formula (I).

When R² is methyl the stereochemistry at the carbon to which it is attached is preferably in the (R)-configuration.

R³ is preferably hydrogen, fluoro or chloro, more preferably hydrogen or fluoro.

R⁵ is preferably:

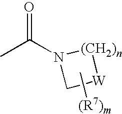

n is preferably 2.

W is preferably CH₂.

Y is preferably —CH₂— or —CHMe-.

The stereochemistry of the compounds of formula (I) is preferably as shown below:

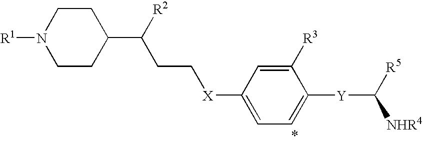

While the preferred groups for each variable have generally been listed above separately for each variable, preferred compounds of this invention include those in which several or each variable in formula (I) is selected from the preferred groups for each variable. Therefore, this invention is intended to include all combinations of preferred listed groups.

Representative compounds of the invention which may be mentioned are those provided in the Examples as the free base or a pharmaceutically acceptable salt thereof.

The molecular weight of the compounds of the invention is preferably less than 800, more preferably less than 600, especially less than 500.

As used herein, unless stated otherwise, “alkyl” means carbon chains which may be linear or branched. Examples of alkyl groups include ethyl, propyl, isopropyl, butyl, sec- and tert-butyl.

The term “heteroaryl” rings means 5- or 6-membered N-containing heteroaryl rings containing up to 2 additional heteroatoms selected from N, O and S. Examples of such heteroaryl rings are pyrrolyl, pyrazolyl, imidazolyl, oxazolyl, isoxazolyl, thiazolyl, isothiazolyl, triazolyl, oxadiazolyl, thiadiazolyl, pyridinyl, pyridazinyl, pyrimidinyl, pyrazinyl and triazinyl.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The present invention includes all stereoisomers of the compounds of the invention and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of the invention exists, the present invention includes any possible tautomers and pharmaceutically acceptable salts thereof, and mixtures thereof, except where specifically drawn or stated otherwise.

When the compound of the invention and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of the invention are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, wherein R¹, R², R³, R⁵, R⁶, R⁷, X, Y, W, m and n are as defined for formula (I). R⁴ is as defined for formula (I) or can be a protecting group, for example para-OMe phenyl. R⁸ is one or more fluoro, chloro, cyano or methyl groups, PG is a protecting group, Ak is C₁₋₂ alkyl, Hal is halo and G is 5- or 6-membered heteroaryl.

Compounds of formula (I) where X═O and R⁵ is an amide as defined as above, can be synthesized as outlined in Scheme 1. Compounds of formula (IV) can be synthesized by reaction of an alcohol of formula (II) with a phenol of formula (III) under, for example, Mitsunobu conditions using azodicarboxylic dipiperidide. Alternatively, compounds of formula (IV) can be synthesized by reaction of an alkoxide of a compound of formula (III) with a mesylate of a compound of formula (II), in a suitable solvent such as DMF. Saponification of the ester, followed by amide bond formation and deprotection of the amine functionality, using standard conditions well known to those with skill in the art, yields compounds of formula (I) as described above. Chiral compounds of formula (I) can be synthesized through use of a chiral building block of formula (III) or through isolation of the desired enantiomer by chiral-HPLC at one of the stages of the synthesis.

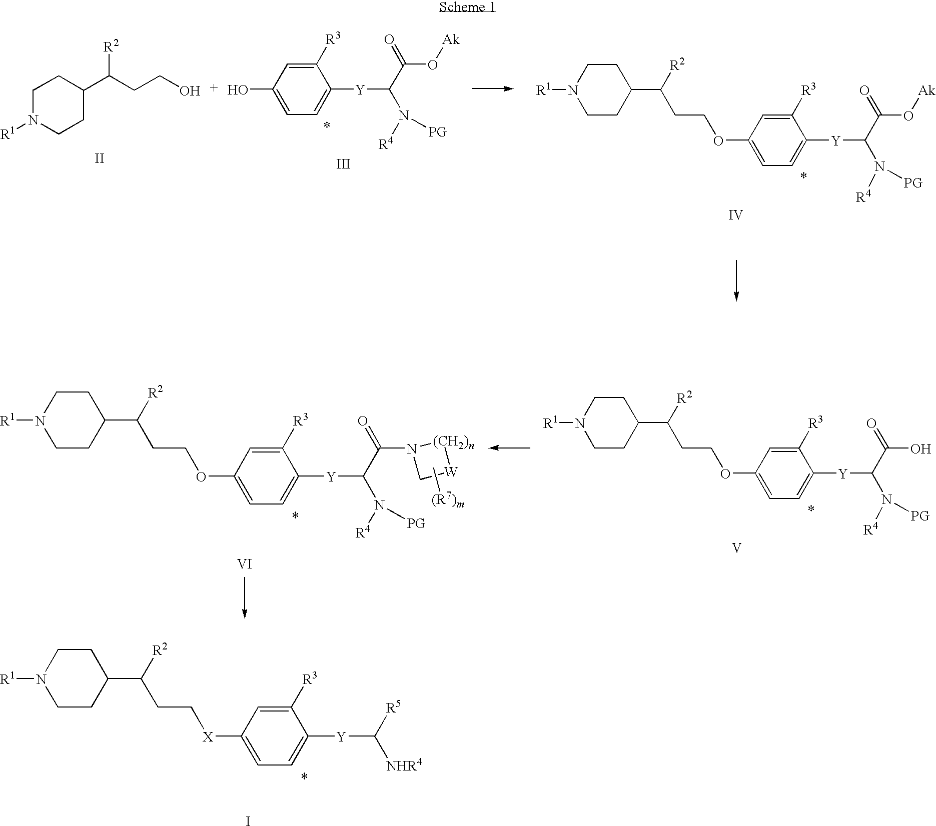

Unichiral building blocks for compounds of formula (II), where R² is Me, can be readily prepared from known compounds (Scheme 2). For example, the ethyl ester of compound (VII) where PG is tert-butoxy carbonyl (Boc) has been previously reported (U.S. Pat. No. 6,518,423). Saponification and hydrogenation, under standard conditions, will yield the racemic compound of formula (VIII). Chiral reduction of the alkenoic acid (VII) under suitable conditions, such as a hydrogenation in the presence of a chiral catalyst, yields compounds of formula (VIII) in high enantiomeric excess. An example of a suitable catalyst is [Rh(norbornadiene)₂]BF₄ and (5)-1-[(R)-2-(di-tert-butylphosphino)ferrocenyl]ethylbis(2-methylphenyl)phosphine. Compounds of formula (IX) can then be obtained by reduction of the carboxylic acids of formula (VIII) under standard conditions, for example borane in a suitable solvent such as THF.

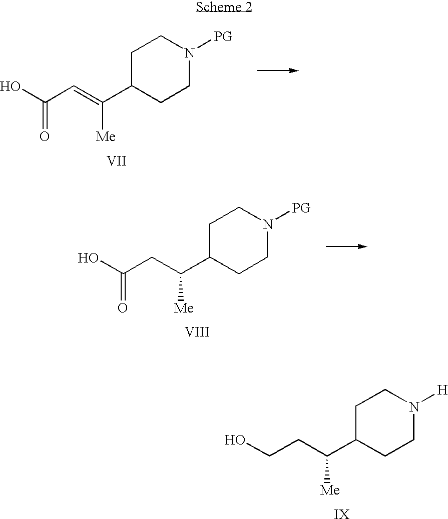

Building blocks of formula (II), where R¹ is a substituted oxadiazole, can be prepared as outlined in Scheme 3. The compound of formula (X) where R²═H is a known compound (Siegel, M. G. et al. Tetrahedron 1999, 55, 11619-11639). Compounds of formula (XI) can be prepared from compounds of formula (X) under standard conditions. For example, treatment of compounds of formula (X) with cyanogen bromide followed by condensation of the resultant cyanamide (XI) with a compound of formula (XII) under standard conditions yields compounds of formula (II) where R¹ is a substituted oxadiazole. Compounds of formula (XII) are either commercially available, or readily prepared from the corresponding carboxylic acids using well known techniques. Alternatively, synthesis of the regioisomeric oxadiazole can be achieved by heating compounds of formula (XI) with hydroxylamine to give N-hydroxyguanidines of formula (XIII) that may be condensed with a carboxylic acid of formula (XIV) under suitable conditions. Acids of formula (XIV) are commercially available.

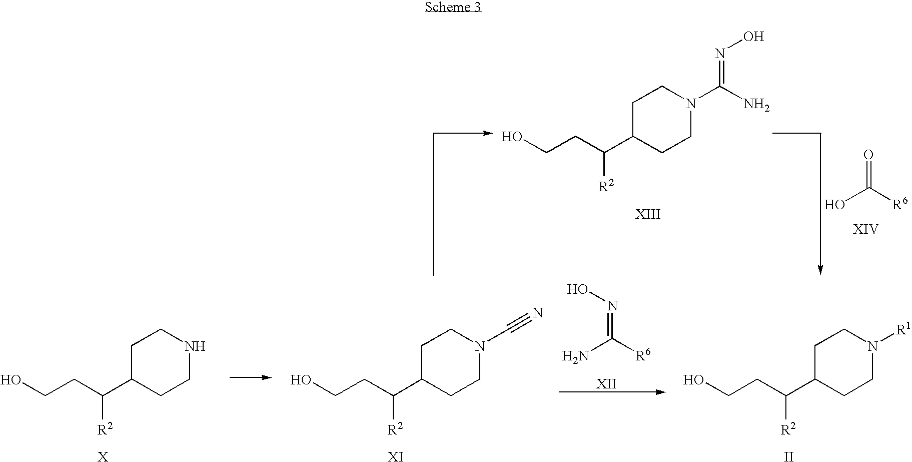

Compounds of formula (II) where R¹ is 5- or 6-membered heteroaryl may also be prepared by condensation of amine (X) with a 5- or 6-membered heteroaryl halide of formula (XV), as illustrated in Scheme 4 (Buscemi, S. et al. JCS Perkin I. Org. and Bioorg. Chem., 1988, 1313 and Adembri, G, et al. JCS Perkin I. Org. and Bioorg. Chem., 1981, 1703).

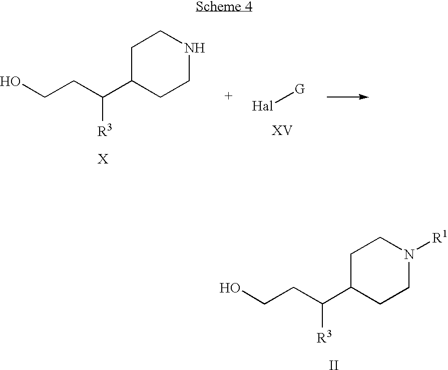

Compounds of formula (III) are either commercially available, are known compounds or can be prepared as outlined in Schemes 5-8.

For example, the compound of formula (III) with S stereochemistry where R³ and R⁴ are hydrogen, Y is a CH₂, Ak is Me and PG is Boc is commercially available. The compound of formula (III) with (S)-stereochemistry where R³ and R⁴ are hydrogen, Y is a bond, Ak is Me and PG is Boc, is a known compound (Grimm, J. B. et al. Tett. Letts., 2007, 48(26), 4509). The compound of formula (III) where R³ is hydrogen, Y is CH₂ and R⁴ is —CH₂— linked to position * on the phenyl ring to form a fused 6-membered N-containing heterocycle is a known compound (Wang, Aihua et al, US20020061885).

Specifically, chiral compounds of formula (III) where R³ is fluorine, Y is a bond and R⁴ is para-OMe phenyl can be prepared as outlined in Scheme 5. The compound of formula (XVIII) can be prepared by reaction of the aldehyde of formula (XVI) with 4-methoxyphenyl-amine under standard reductive amination conditions, followed by protection of the phenol with tert-butylchlorodimethylsilane. Protection of the amine with, for example, di-tert-butyl dicarbonate, under standard conditions, provides compounds of the formula (XIX). Subsequent reaction with lithium-2-butanide and (−)-sparteine, followed by the addition of methylchloroformate affords compounds of the formula (XX). Deprotection of the phenol with, for example, TBAF provides compounds of formula (III) as described above.

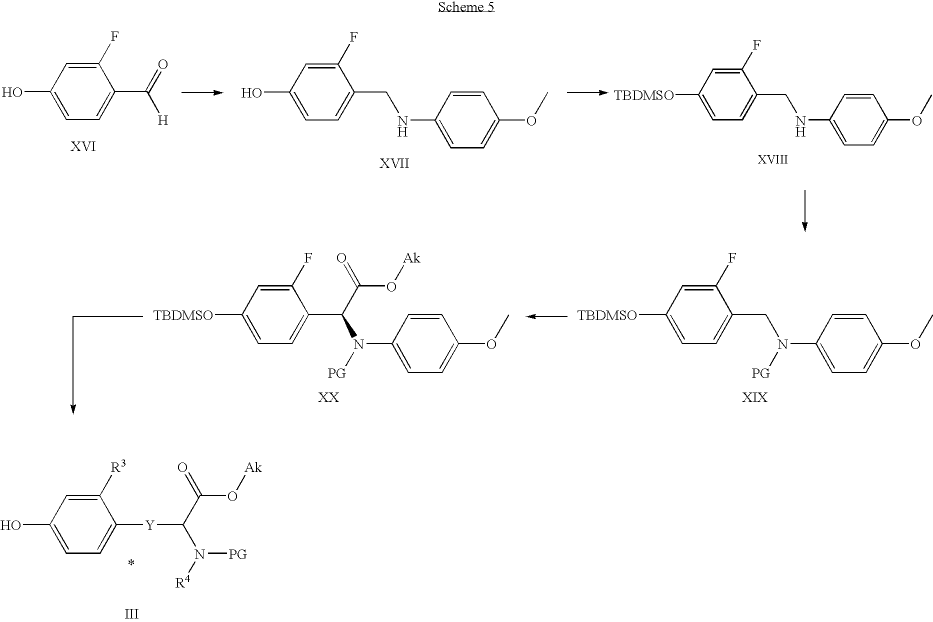

Specifically, compounds of formula (III) where R³ is either fluorine or methyl, Y is a bond and R⁴ is hydrogen can be prepared as outlined in Scheme 6. Compounds of formula (XXII) can be prepared by reaction of an aldehyde of formula (XXI) with sodium cyanide and ammonia. Hydrolysis, under standard acidic conditions, followed by protection of the amine with, for example, di-tert-butyldicarbonate, affords compounds of the formula (XXIV). Subsequent formation of an alkyl ester, using standard conditions well known to those with skill in the art, yields compounds of formula (III) as described above.

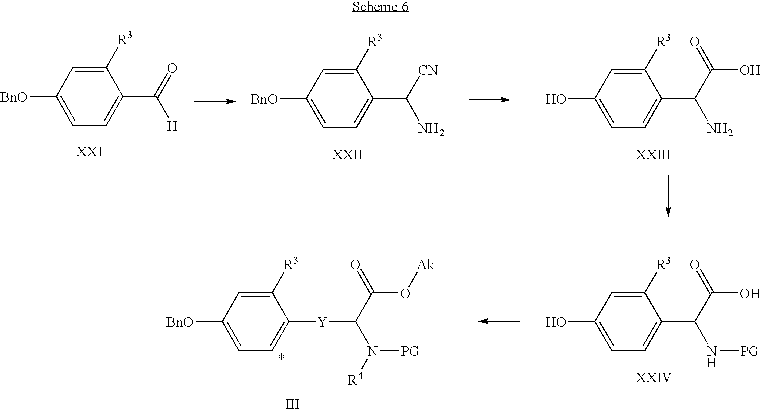

Specifically, compounds of formula (III) where R³ is fluorine, Y is CH₂ and R⁴ is hydrogen can be prepared as outlined in Scheme 7. Compounds of formula (XXVI) can be prepared by reaction of 2-fluoro-4-methoxybenzaldehyde (XXV) with sodium acetate and acetylaminoacetic acid at 120° C. in acetic anhydride. Reduction of the resulting alkenoic acid (XXVI), under standard conditions, affords a racemic compound of formula (XXVII). Reduction of the alkenoic acid (XXVI) with a chiral catalyst, such as [Rh(cod)(PP)]OTf and (S, S)-Et-Duphos, affords a compound of formula (XXVII) in high enantiomeric excess. Removal of the acetyl group, under standard acidic conditions, followed by protection of the amine group with, for example, di-tert-butyldicarbonate yields compounds of formula (XXIX). Subsequent formation of an alkyl ester, using conditions well known to those with skill in the art, yields compounds of formula (III) as described above.

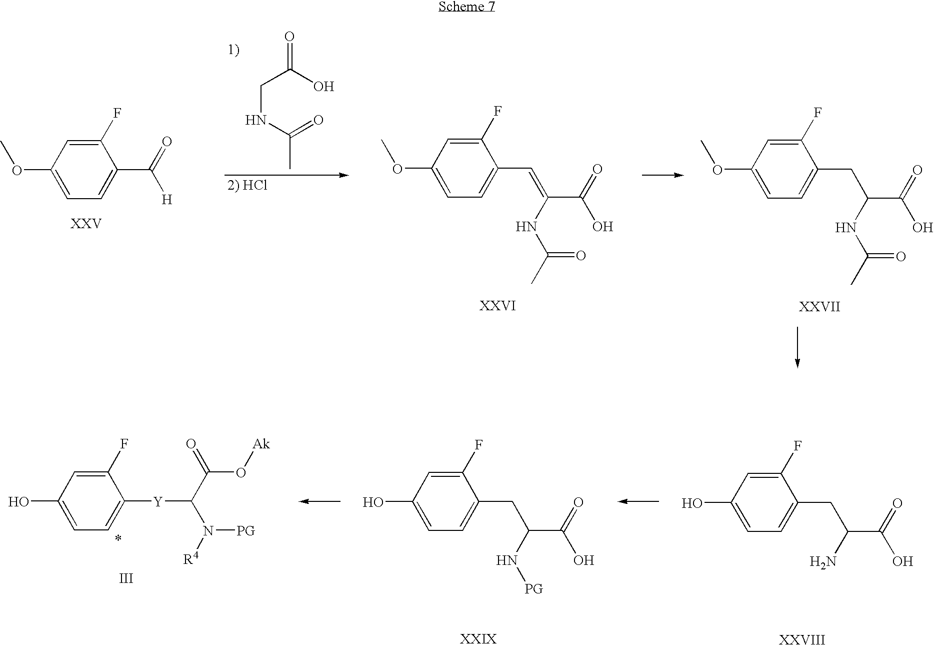

Specifically, chiral compounds of formula (III) where R³ is fluorine, Y is CHMe and R⁴ is hydrogen can be prepared as outlined in Scheme 8. The compound of formula (XXXI) can be synthesized by reaction of 4-benzyloxy-2-fluorobenzaldehyde (XXX) with (triphenyl-lambda*5*-phospanylidene)acetic acid methyl ester in a suitable solvent, such as THF, under reflux conditions. Saponification, followed by activation of the resulting carboxylic acid (XXXII) with, for example, pivaloyl chloride, followed by reaction with (R)-(−)-4-phenyl-2-oxazolidinone which has been deprotonated with a suitable base, such as n-butyllithium, affords the compound of formula (XXXIII). Reaction with dimethyl sulfide, methyl magnesium bromide and copper (I) bromide-dimethyl sulfide in a suitable solvent, such as THF, yields the compound of formula (XXXIV). Subsequent reaction with dibutylborontriflate, followed by reaction with N,N,N′,N′-tetramethylguanidinium azide, affords the compound of formula (XXXVI). Removal of the phenyloxazolidin-2-one group, with hydrogen peroxide and sodium hydroxide, gives the compound of formula (XXXVII). Reduction, under standard conditions, followed by protection of the resulting amine group with, for example, di-tert-butyldicarbonate, affords compounds of the formula (XXXIX). Subsequent formation of an alkyl ester, using conditions well known to those with skill in the art, yields compounds of formula (III) as described above.

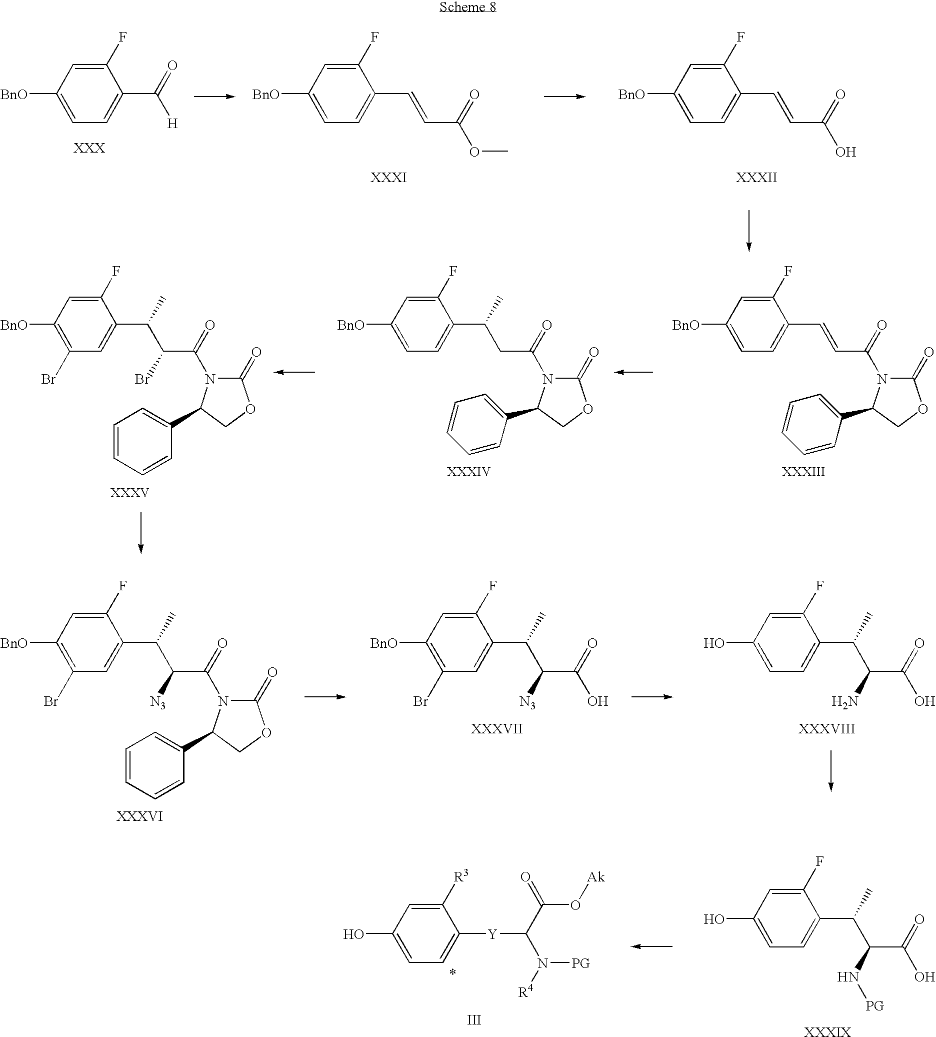

Specifically, chiral compounds of formula (I) where X═O, R³ is methyl, R⁴ is hydrogen, R⁵ is an amide as defined as above, and Y is CH₂ can be prepared as outlined in Scheme 9. Compounds of formula (XLI) can be synthesized by reaction of an alcohol of formula (II) with a phenol of formula (XL) under, for example, Mitsunobu conditions using azodicarboxylic dipiperidide. Alternatively, compounds of formula (XLI) can be synthesized by reaction of an alkoxide of a compound of formula (XL) with a mesylate of a compound of formula (II), in a suitable solvent such as DMF. Compounds of formula (XLI) can be converted to compounds of formula (XLIII) by reaction with an appropriate organozinc reagent, formed from an iodide of formula (XLII), using standard palladium coupling conditions, such as palladium acetate and tributylphosphine. An amide coupling reaction, followed by deprotection of the amine, using standard conditions well known to those with skill in the art, affords compounds of formula (I) as described above.

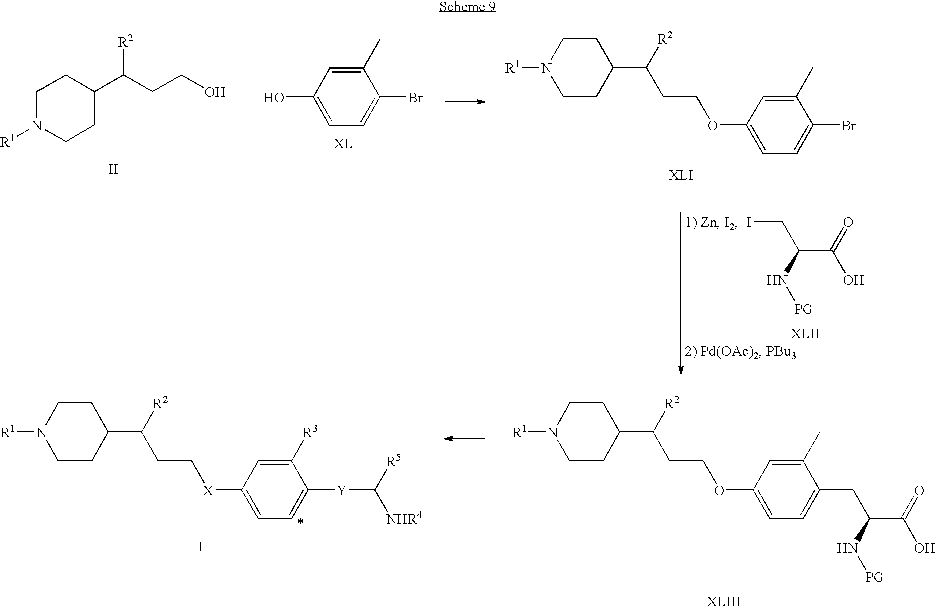

Specifically, compounds of formula (I) where X═O, R⁴ is hydrogen, R⁵ is a benzyl group as defined above and Y is a bond can be prepared as outlined in Scheme 10. Compounds of formula (XLV) can be prepared by reaction of an alcohol of formula (II) with a phenol of formula (XLIV) under, for example, Mitsunobu conditions using azodicarboxylic dipiperidide. Alternatively, compounds of formula (XLV) can be synthesized by reaction of an alkoxide of a compound of formula (XLIV) with a mesylate of a compound of formula (II), in a suitable solvent such as DMF. Subsequent reaction of compounds of formula (XLV) with LiHMDS, followed by reaction of the resultant imine with a Grignard reagent of formula (XLVI) affords compounds of formula (I) as described above.

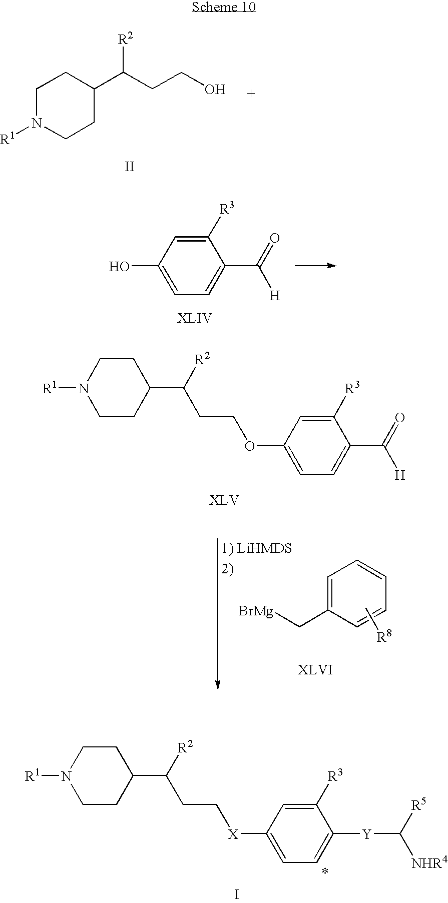

Specifically, chiral compounds of formula (I) where X and Y are CH₂, R³ is fluorine, R⁴ is hydrogen and R⁵ is an amide as defined as above, can be prepared as outlined in Scheme 11. An alkyne of formula (XLVIII) can be prepared from an alcohol of formula (II) by oxidation to the corresponding aldheyde (XLVII) using a standard oxidizing reagent, such as Dess-Martin Periodinane, and subsequent reaction of the aldehyde of formula (XLVII) with a suitable base, such as nBuLi, followed by reaction with trimethylsilyldiazomethane. A triflate of formula (L) can be synthesized from a phenol of formula (XXIX) via formation of an alkyl ester of formula (XLIX), using standard conditions well known to those with skill in the art, followed by formation of the triflate using, for example, N-phenyl-bis(trifluoromethylsulfonimide). Compounds of formula (LI) can be prepared from a compound of formula (XLVIII) and a compound of formula (L) via standard Sonogashira coupling conditions. Saponification of the ester, followed by amide bond formation, using conditions well known to those with skill in the art, provides compounds of formula (LIII). Reduction of the alkyne and subsequent deprotection of the amine functionality, using standard conditions well known to those with skill in the art, yields compounds of formula (I) as described above.

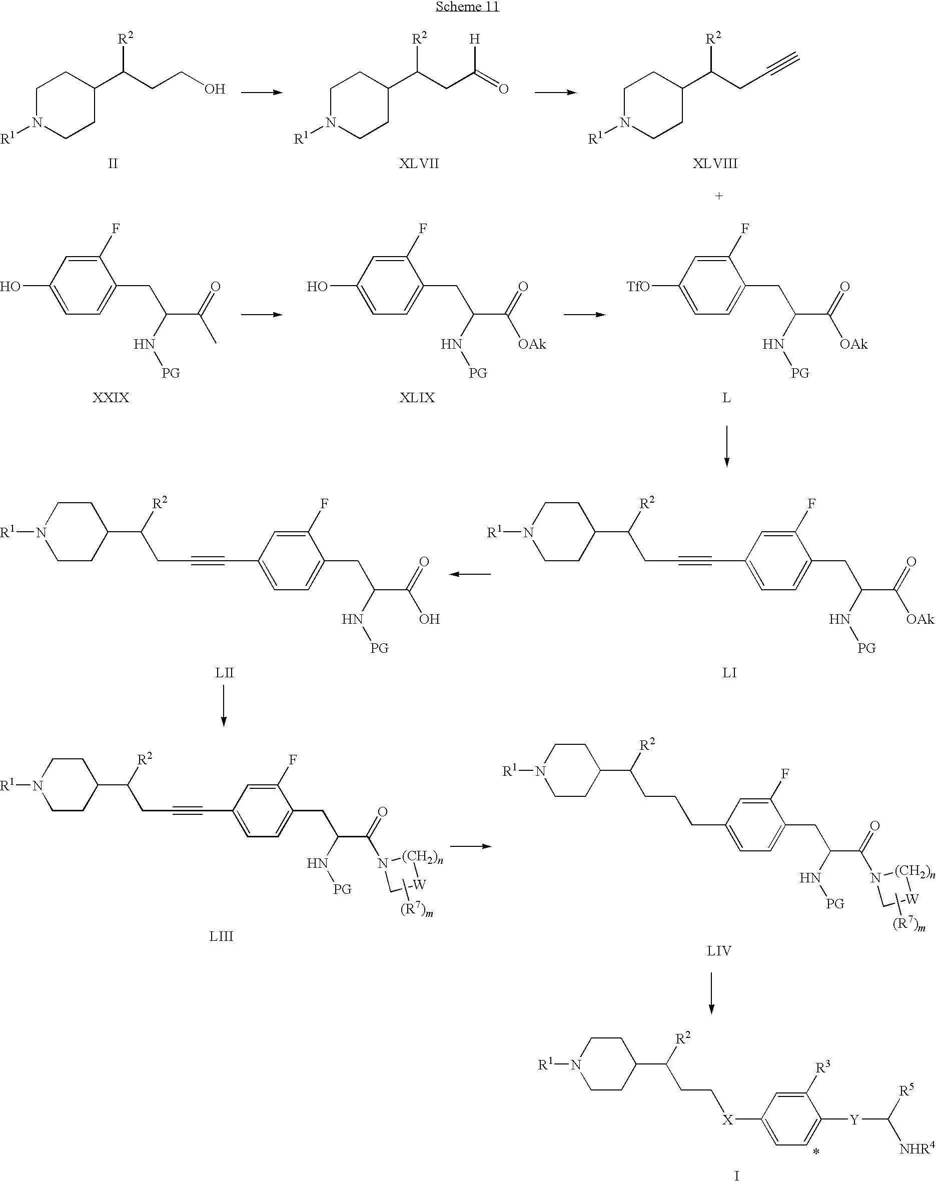

Other compounds of formula (I) may be prepared by methods analogous to those described above or by methods known per se. Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

The processes for the production of the compounds of formula (I) and intermediates thereto as described above are also included as further aspects of the present invention.

Any novel intermediates as defined in the Schemes above or in the Examples, are also included within the scope of the invention. Therefore according to a further aspect of the invention there is provided a compound of any one of formulae (IV), (V), (VI), (XLIII), (XLV), (LI), (LII), (LIII) or (LIV) as defined above. The preferred groups for variables recited above in relation to the compounds of formula (I) also apply to the intermediates compounds.

As indicated above the compounds of the invention are useful as dual GPR119 agonists/DPP-IV inhibitors, e.g. for the treatment and/or prophylaxis of diabetes. For such use the compounds of the invention will generally be administered in the form of a pharmaceutical composition.

The invention also provides a compound of the invention, or a pharmaceutically acceptable salt thereof, for use as a pharmaceutical.

The invention also provides a pharmaceutical composition comprising a compound of the invention, in combination with a pharmaceutically acceptable carrier.

Preferably the composition is comprised of a pharmaceutically acceptable carrier and a non-toxic therapeutically effective amount of a compound of the invention, or a pharmaceutically acceptable salt thereof.

Moreover, the invention also provides a pharmaceutical composition for the treatment of disease by modulating GPR119 and DPP-IV, resulting in the prophylactic or therapeutic treatment of diabetes, comprising a pharmaceutically acceptable carrier and a non-toxic therapeutically effective amount of compound of the invention, or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of the invention, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of the invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of the invention may be used in the treatment of diseases or conditions in which GPR119 and DPP-IV play a role.

Thus the invention also provides a method for the treatment of a disease or condition in which GPR119 and DPP-IV play a role comprising a step of administering to a subject in need thereof an effective amount of a compound of the invention, or a pharmaceutically acceptable salt thereof. Such diseases or conditions diabetes, obesity, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The invention also provides a method for the treatment of type II diabetes, comprising a step of administering to a patient in need thereof an effective amount of a compound of the invention, or a pharmaceutically acceptable salt thereof.

The invention also provides a method for the treatment of obesity, metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of the invention, or a pharmaceutically acceptable salt thereof.

The invention also provides a compound of the invention, or a pharmaceutically acceptable salt thereof, for use in the treatment of a condition as defined above.

The invention also provides the use of a compound of the invention, or a pharmaceutically acceptable salt thereof, in the manufacture of a medicament for the treatment of a condition as defined above.

In the methods of the invention the term “treatment” includes both therapeutic and prophylactic treatment.

The compounds of the invention may exhibit advantageous properties compared to known compounds or combination therapies for the treatment of diabetes.

The compounds of the invention, or pharmaceutically acceptable salts thereof, may be administered alone or in combination with one or more other therapeutically active compounds. The other therapeutically active compounds may be for the treatment of the same disease or condition as the compounds of the invention or a different disease or condition. The therapeutically active compounds may be administered simultaneously, sequentially or separately.

The compounds of the invention may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, RXR agonists, fatty acid oxidation inhibitors, α-glucosidase inhibitors, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of the invention, or a pharmaceutically acceptable salt thereof, and at least one other agent represents a further aspect of the invention.

The present invention also provides a method for the treatment of diabetes in a mammal, such as a human, which method comprises administering an effective amount of a compound of the invention, or a pharmaceutically acceptable salt thereof, and another agent, to a mammal in need thereof.

The invention also provides the use of a compound of the invention, or a pharmaceutically acceptable salt thereof, and another agent for the treatment of diabetes.

The invention also provides the use of a compound of the invention, or a pharmaceutically acceptable salt thereof, in the manufacture of a medicament for use in combination with another agent, for the treatment of diabetes.

The compound of the invention, or a pharmaceutically acceptable salt thereof, and the other agent(s) may be co-administered or administered sequentially or separately.

Co-administration includes administration of a formulation which includes both the compound of the invention, or a pharmaceutically acceptable salt thereof, and the other agent(s), or the simultaneous or separate administration of different formulations of each agent. Where the pharmacological profiles of the compound of the invention, or a pharmaceutically acceptable salt thereof, and the other agent(s) allow it, coadministration of the two agents may be preferred.

The invention also provides the use of a compound of the invention, or a pharmaceutically acceptable salt thereof, and another agent in the manufacture of a medicament for the treatment of diabetes.

The invention also provides a pharmaceutical composition comprising a compound of the invention, or a pharmaceutically acceptable salt thereof, and another antiobesity agent, and a pharmaceutically acceptable carrier. The invention also encompasses the use of such compositions in the methods described above.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### Examples

### Materials and Methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O—CH₃CN solution containing 0.1% HCO₂H over 6 min with UV detection at 220 nm. Gradient information: 0.0-0.3 min 100% H₂O; 0.3-4.25 min: Ramp up to 10% H₂O-90% CH₃CN; 4.25-4.4 min: Ramp up to 100% CH₃CN; 4.4-4.9 min: Hold at 100% CH₃CN; 4.9-6.0 min: Return to 100% H₂O. The mass spectra were obtained using an electrospray ionisation source in either the positive (ES⁺) or negative (ES⁻) ion modes.

LCMS data (method 2) were obtained as follows: Chromolith SpeedROD column (4.6×50.0 monolith, flow rate=3.0 mL/min) eluting with a H₂O—CH₃CN solution containing 0.1% TFA over 3 min with UV detection at 220 nm. Gradient information: 0-2 min: 99% H₂O 1% MeCN to 100% MeCN; 2-3 min: Hold at 100% CH₃CN; The mass spectra were obtained using an electrospray ionisation source in the positive (ES⁺) mode.

Chiral-HPLC was performed on a Daicel chiral pack IA 250×20 mm, 5 μM column.

Abbreviations and acronyms: Ac: Acetyl; ADDP: Azodicarboxylic dipiperidide; t-Bu: tert-Butyl; DBU: 1,8-Diazabicyclo[5.4.0]undec-7-ene; DCE: 1,2-Dichloroethane; DCM: Dichloromethane; DIPEA: N,N-Diisopropylethylamine; DMF: Dimethylformamide; EDCI: 1-(3-Dimethylaminopropyl)-3-ethylcarbodiimide hydrochloride; Et: Ethyl; h: hour(s); min: minute/s; HATU: O-(7-Azabenzotriazol-1-yl)-N,N,N′,N′-tetramethyluronium hexafluorophosphate; HPLC: High performance liquid chromatography; HOBt: 1-Hydroxybenzotriazole; IH: Isohexane; LiHMDS: Lithium bis(trimethylsilyl)amide; Me: Methyl; MeCN: Acetonitrile; MP: Macroporous Polystyrene; PBu₃: tri-tent-butyl phosphine; PE-AX column: silica based quaternary amine column; RP: Reverse Phase; RT: Retention time; TBAF: Tetra-butyl ammonium fluoride; THF: Tetrahydrofuran; TFA: Trifluoroacetic acid; TMS: Trimethylsilyl.

The syntheses of the following compounds have been described elsewhere: 4-benzyloxy-2-fluorobenzaldehyde: WO2008/052658; tert-butyl 4-((E)-2-ethoxycarbonyl-1-methylvinyl)piperidine-1-carboxylate: U.S. Pat. No. 6,518,423; 3-fluoro-4-hydroxymethyl-phenol: WO01/20995; 4-(3-hydroxypropyl)piperidine-1-carboxylic acid isopropyl ester: WO2007/003962. All other compounds were available from commercial sources.

### Preparation 1: 4-(3-Hydroxypropyl)piperidine-1-carbonitrile

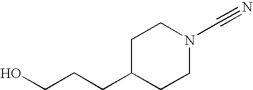

A slurry of NaHCO₃ (35.2 g, 0.42 mol) in H₂O (70 mL) was added to a stirred solution of 3-piperidin-4-ylpropan-1-ol (20.0 g, 0.14 mol) in DCM at 0° C. A solution of BrCN (17.8 g, 170 mmol) in DCM (19 mL) was added to the reaction over 1 min, then stiffing was continued at 0° C. for 0.5 h. The reaction was then stirred at 20° C. for 2 h, before being washed with saturated aqueous NaHCO₃ solution and brine. The DCM solution was dried (MgSO₄), filtered and concentrated in vacuo to furnish an oil that was dissolved in a small amount of DCM, before being filtered through a SiO₂ pad, eluting with EtOAc. The filtrate was concentrated under reduced pressure to afford the title compound: m/z (ES⁺)=169.1 [M+H]⁺.

### Preparation 2: 3-[1-(3-Isopropyl[1,2,4]oxadiazol-5-yl)piperidin-4-yl]propan-1-ol

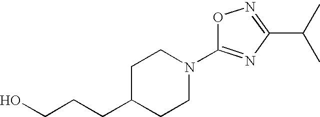

ZnCl₂ (1M in Et₂O, 145 mL, 145 mmol) was added over 20 min to a stirred solution of 4-(3-hydroxypropyl)piperidine-1-carbonitrile (Preparation 1, 20.3 g, 121 mmol) and N-hydroxyisobutyramidine (14.8 g, 145 mmol) in EtOAc (290 mL) and THF (270 mL). After 2 h, the white precipitate that had formed was collected and washed with THF-EtOAc (1:1, 50 mL). This precipitate was dissolved in EtOH (550 mL) and 12M HCl (70 mL), then the solution was stirred with heating to 70° C. for 16 h. The EtOH was removed in vacuo, the remainder was diluted with H₂O, then the pH was adjusted to pH 7 with solid NaHCO₃. The mixture was extracted with EtOAc (3×), then the combined extracts were washed with brine, before being dried (MgSO₄). Filtration and solvent removal furnished the title compound: m/z (ES⁺)=254.1 [M+H]⁺.

### Preparation 3: Methanesulfonic acid 3-[1-(3-isopropyl-[1,2,4]oxadiazol-5-yl)piperidin-4-yl]-propyl ester

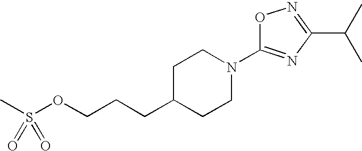

Methanesulfonyl chloride (1.64 mL, 21.2 mmol) in DCM (5 mL) was added dropwise to a solution of 3-[1-(3-isopropyl [1,2,4]oxadiazol-5-yl)piperidin-4-yl]propan-1-ol (Preparation 2, 4.46 g, 17.6 mmol) and NEt₃ (4.90 mL, 35.3 mmol) in DCM (35 mL) at 0° C. The reaction mixture was stirred at ambient temperature for 0.5 h, then partitioned between EtOAc (250 mL) and 0.5M HCl (150 mL). The organic layer was separated, washed with H₂O, saturated aqueous NaHCO₃ solution and brine, before being dried (MgSO₄), filtered, and concentrated in vacuo to afford the title compound: RT=3.32 min; m/z (ES⁺)=332.08 [M+H]⁺.

### Preparation 4: tert-Butyl 4-((E)-2-carboxy-1-methylvinyl)piperidine-1-carboxylate

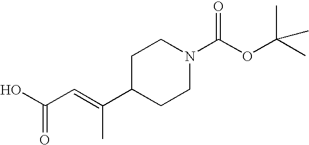

A solution of tert-butyl 4-((E)-2-ethoxycarbonyl-1-methylvinyl)piperidine-1-carboxylate (18.7 g, 62.9 mmol) in MeOH (90 mL) and H₂O (25 mL) was treated with 2M NaOH (94.5 mL, 189 mmol). The reaction was stirred for 16 h, the MeOH was removed under reduced pressure, then the remainder was partitioned between EtOAc and H₂O. The aqueous layer was separated and acidified to pH 2 with 12M HCl, before being extracted with EtOAc (2×). The organic extracts were washed with brine, dried (MgSO₄), filtered, and concentrated in vacuo, then the remainder was recrystallized from EtOAc-IH to provide the title compound: m/z (ES⁻)=268.3 [M−H]⁻.

### Preparation 5: tert-Butyl 4-((R)-2-carboxy-1-methylethyl)piperidine-1-carboxylate

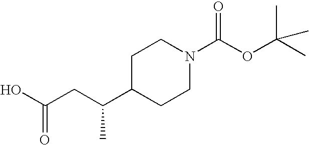

tert-Butyl 4-((E)-2-carboxy-1-methylvinyl)piperidine-1-carboxylate (Preparation 4, 130 g, 483 mmol) was placed in a hydrogenation flask under an Ar atmosphere, then degassed MeOH (400 mL) was added. [Rh(norbornadiene)₂]BF₄ (1.80 g, 4.81 mmol) and (S)-1-[(R)-2-(di-tert-butylphosphino)ferrocenyl]ethylbis(2-methylphenyl)phosphine (2.90 g, 5.08 mmol) were placed in a separate Schlenk flask under Ar, before being treated with degassed MeOH (200 mL). This catalyst mixture was stirred for 15 min at ambient temperature, before being transferred via cannula into the hydrogenation flask. The Schlenk flask was rinsed with more degassed MeOH (100 mL). These washings were transferred to the hydrogenation flask, then more degassed MeOH (300 mL) was added. The hydrogenation flask was sealed, the Ar replaced by H₂, and the pressure set to 1.05 bar. The reaction mixture was heated to 35° C., and stirring/shaking was started. After 48 h, the reaction was stopped and a representative sample of the reaction mixture was analyzed by HPLC and ¹H NMR. The conversion was 100% and the enantiomeric purity of the crude (R)-acid was 98.2%, as ascertained by the following HPLC method: Column: CHIRALPAK AD-H (previously used with CF₃CO₂H-containing solvents) 4.6×250 mm; Solvent: C₆H₁₄-iPrOH (97:3 isocratic); Temperature: 20° C.; Flow rate: 1 mL/min; UV-detection (210, 230 nm); Sample: 100 μL reaction solution dissolved with 1 mL MeOH. Retention times: (S)-acid: 19.3 min, (R)-acid: 20.6 min, starting enoic acid: 22.1 min. Isolation procedure: The MeOH was evaporated, then the crude hydrogenation product was dissolved in t-BuOMe and extracted with aqueous NaOH. The aqueous phase was added to a mixture of 1M HCl and EtOAc. The aqueous phase was extracted further with EtOAc, then the combined organic extracts were washed with brine and dried (MgSO₄). The title compound was isolated following filtration and complete removal of the solvent.

### Preparation 6: tert-Butyl 4-((R)-3-hydroxy-1-methylpropyl)piperidine-1-carboxylate

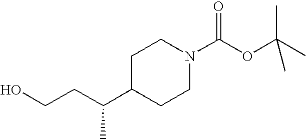

BH₃.THF (1M, 15.7 mL, 15.7 mmol) was added dropwise over 5 min to a stirred solution of tert-butyl 4-((R)-2-carboxy-1-methylethyl)piperidine-1-carboxylate (Preparation 5, 1.70 g, 6.30 mmol) in anhydrous THF at 0° C. After 1 h, the reaction was treated with Et₂O, then with 2M HCl. The organic layer was washed with brine, before being dried (Na₂SO₄). Filtration, solvent evaporation, and column chromatography (EtOAc-CH₂Cl₂, 1:3) provided the title compound: RT=3.17 min; m/z (ES⁺)=258.1 [M+H]⁺.

### Preparation 7: 4-((R)-3-Hydroxy-1-methylpropyl)piperidine-1-carbonitrile

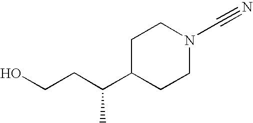

A mixture of tert-butyl 4-((R)-3-hydroxy-1-methylpropyl)piperidine-1-carboxylate (Preparation 6, 6.20 g, 14.9 mmol) and 4M HCl in dioxane (10 mL) were stirred at ambient temperature. After 3 h, the solvents were removed under reduced pressure to furnish the hydrochloride salt of (R)-3-piperidin-4-yl-butan-1-ol: δ_{H} ({CD₃}₂SO) 0.83 (d, 3H), 1.19-1.28 (m, 1H), 1.38-1.59 (m, 5H), 1.64-1.76 (m, 2H), 2.75-2.87 (m, 2H), 3.20-3.30 (m, 2H), 3.35-3.60 (m, 4H). A stirred mixture of this compound (930 mg, 4.80 mmol) and NaHCO₃ (1.61 g, 19.2 mmol) in DCM-H₂O (4:1, 15 mL) at 0° C. was treated with a solution of BrCN (610 mg, 5.80 mmol) in DCM (2 mL). The reaction was stirred at 20° C. for 2 h, before being partitioned between H₂O and DCM. The organic phase was separated and dried (MgSO₄). Filtration, solvent evaporation, and column chromatography (EtOAc) provided the title compound: RT=2.45 min; m/z (ES⁺)=183.1 [M+H]⁺.

### Preparation 8: (R)-3-[1-(3-Isopropyl-[1,2,4]oxadiazol-5-yl)piperidin-4-yl]butan-1-ol

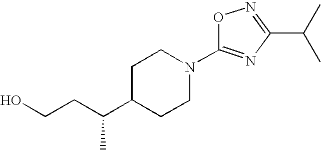

Condensation of 4-((R)-3-hydroxy-1-methylpropyl)piperidine-1-carbonitrile (Preparation 7, 530 mg, 2.90 mmol) with N-hydroxyisobutyramidine (0.36 g, 3.5 mmol), employing a procedure similar to that outlined in Preparation 2, afforded the title compound: RT=2.92 min; m/z (ES⁺)=268.1 [M+H]⁺.

### Preparation 9: Methanesulfonic acid (R)-3-[1-(3-isopropyl-[1,2,4]oxadiazol-5-yl)piperidin-4-yl]butyl ester

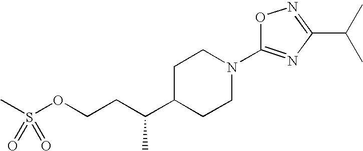

Methanesulfonyl chloride (610 μL, 7.90 mmol) and NEt₃ (2.01 mL, 15.0 mmol) were added to a solution of (R)-3-[1-(3-isopropyl-[1,2,4]oxadiazol-5-yl)piperidin-4-yl]butan-1-ol (Preparation 8, 2.00 g, 7.50 mmol) in DCM (30 mL) at 0° C. After stirring for 10 min, the reaction was diluted with DCM (100 mL) and poured into saturated aqueous NaHCO₃ solution (100 mL). The organic layer was separated, washed with 0.1M HCl (100 mL), dried (MgSO₄), filtered and concentrated in vacuo. Purification by column chromatography (EtOAc-1H, 1:1) afforded the title compound: RT=3.42 min; m/z (ES⁺)=346.1 [M+H]⁺.

### Preparation 10: (S)-2-tert-Butoxycarbonylamino-3-(4-{3-[1-(3-isopropyl-[1,2,4]oxadiazol-5-yl)piperidin-4-yl]propoxy}phenyl)propionic acid

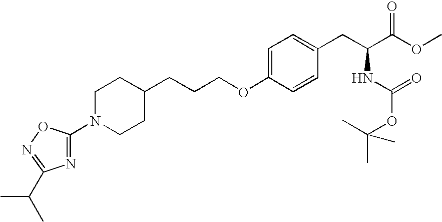

(S)-2-tert-Butoxycarbonylamino-3-(4-hydroxyphenyl)propionic acid methyl ester (100 mg, 340 μmol) and K₂CO₃ (47.0 mg, 340 μmol) were added to a solution of methanesulfonic acid 3-[1-(3-isopropyl-[1,2,4]oxadiazol-5-yl)piperidin-4-yl]propyl ester (Preparation 3, 112 mg, 340 μmol) in acetone (4 mL) and the resulting solution was heated at 56° C. for 16 h. Analysis by chiral HPLC revealed greater than 90% enantiomeric excess. The reaction was stirred at ambient temperature for 24 h, then at 56° C. for 24 h. The reaction mixture was diluted with H₂O (20 mL) and extracted with EtOAc, then the organic extract was washed with 0.2M NaOH, dried (MgSO₄), filtered and concentrated in vacuo. Purification by column chromatography (EtOAc-IH, 1:3) afforded the title compound: RT=4.24 min; m/z (ES⁺)=531.27 [M+H]⁺.

### Preparation 11: (S)-2-tert-Butoxycarbonylamino-3-(4-{3-[1-(3-isopropyl-[1,2,4]oxadiazol-5-yl)piperidin-4-yl]propoxy}phenyl)propionic acid

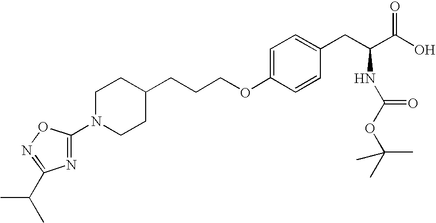

A mixture of LiOH.H₂O (127 mg, 3.03 mmol) and (S)-2-tert-butoxycarbonylamino-3-(4-{3-[1-(3-isopropyl-[1,2,4]oxadiazol-5-yl)piperidin-4-yl]propoxy}phenyl)propionic acid (Preparation 10, 535 mg, 1.01 mmol) in MeOH (10 mL) and H₂O (2 mL) was stirred at 0° C. for 5 h. The MeOH was removed under reduced pressure, then the remainder was diluted with H₂O (20 mL) and acidified to pH 4-5 with citric acid, before being extracted with EtOAc (2×). The combined organic extracts were dried (MgSO₄), filtered and concentrated in vacuo. Analysis by chiral HPLC revealed 90% enantiomeric excess. Purification by column chromatography (EtOAc) afforded the title compound: RT=3.87 min; m/z (ES⁺)=517.27 [M+H]⁺.

### Preparation 12: [(S)-1-(4-{3-[1-(3-Isopropyl-[1,2,4]oxadiazol-5-yl)piperidin-4-yl]propoxy}-benzyl)-2-oxo-2-thiazolidin-3-ylethyl]carbamic acid tert-butyl ester

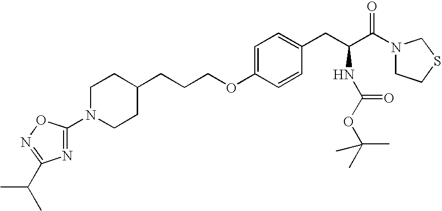

A solution of (S)-2-tert-butoxycarbonylamino-3-(4-{3-[1-(3-isopropyl-[1,2,4]oxadiazol-5-yl)piperidin-4-yl]propoxy}phenyl)propionic acid (Preparation 11, 90 mg, 174 μmol), HOBt.H₂O (40.0 mg, 259 μmol), EDCI (42.0 mg, 218 μmol) and DIPEA (45 μL, 218 μmol) in THF (5 mL) was stirred at ambient temperature for 30 min. Thiazolidine (20.3 μL, 259 μmol) was added and the resulting solution was stirred at ambient temperature for 16 h. The THF was removed in vacuo, then the remainder was dissolved in DCM, washed with dilute aqueous citric acid, saturated aqueous Na₂CO₃ solution and brine, before being dried (MgSO₄), filtered and concentrated in vacuo. Purification by column chromatography (EtOAc-IH, 3:7) afforded the title compound: RT=4.15 min; m/z (ES⁺)=588.14 [M+H]⁺.

### Preparation 13: [(S)-2-((S)-3-Fluoropyrrolidin-1-yl)-1-(4-{3-[1-(3-isopropyl-[1,2,4]oxodiazol-5-yl)piperidin-4-yl]propoxy}benzyl)-2-oxoethyl]carbamic acid tert-butyl ester

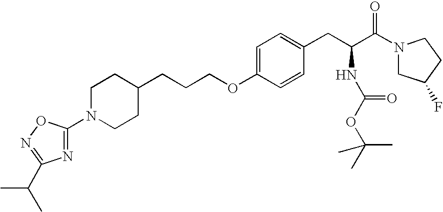

The title compound was synthesized from (S)-2-tert-butoxycarbonylamino-3-(4-{3-[1-(3-isopropyl-[1,2,4]oxadiazol-5-yl)piperidin-4-yl]propoxylphenyl)propionic acid (Preparation 11, 100 mg, 190 μmol) and (S)-3-fluoropyrrolidine hydrochloride (30.0 mg, 240 μmol) employing a procedure similar to that outlined in Preparation 12: RT=3.95 min; m/z (ES⁺)=588.31 [M+H]⁺.

### Preparation 14: (S)-tert-Butoxycarbonylamino-(4-hydroxyphenyl)acetic acid

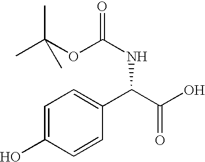

(S)-4-Hydroxyphenylglycine (10.00 g, 59.8 mmol) was added to H₂O (50 mL) and THF (50 mL) at 0° C. under argon. K₂CO₃ (16.40 g, 119.8 mmol) and di-tert-butyl dicarbonate (14.4 g, 66.0 mmol) were added and the reaction was stirred at ambient temperature for 16 h. The THF was removed in vacuo, then the aqueous layer was extracted with EtOAc (50 mL), acidified to pH 4 with citric acid and further extracted with EtOAc (2×50 mL). The combined organic extracts were dried (MgSO₄), filtered and concentrated in vacuo, azeotroping several times with DCM to afford the title compound: RT=2.55 min, m/z (ES⁺)=268.1 8 M+H]⁺.

### Preparation 15: (S)-tert-Butoxycarbonylamino-(4-hydroxyphenyl)acetic acid methyl ester

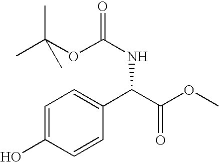

Trimethylsilyldiazomethane (5.40 mL, 7.62 mmol) was added dropwise to a solution of (S)-tert-butoxycarbonylamino-(4-hydroxyphenyl)acetic acid (Preparation 14, 2.00 g, 7.48 mmol) in toluene:MeOH (4:1, 50 mL). The clear solution turned yellow and the MeOH was removed in vacuo. The remainder was diluted with EtOAc (100 mL), washed with H₂O (50 mL), saturated NaHCO₃ solution (50 mL) and brine (50 mL) before being dried (MgSO₄), filtered and concentrated in vacuo, azeotroping several times with Et₂O, to afford the title compound: RT=2.98 min, m/z (ES⁺)=282.1 [M+H]⁺.

### Preparation 16: tert-Butoxycarbonylamino-(4-{3-[1-(3-isopropyl-[1,2,4]oxadiazol-5-yl)-piperidin-4-yl]propoxylphenyl)acetic acid methyl ester

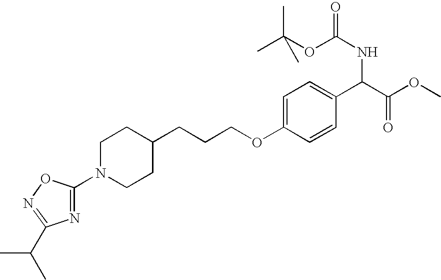

(S)-tert-Butoxycarbonylamino-(4-hydroxyphenyl)acetic acid methyl ester (Preparation 15, 1.00 g, 3.55 mmol) was added to a solution of methanesulfonic acid 3-[1-(3-isopropyl-[1,2,4]oxadiazol-5-yl)piperidin-4-yl]propyl ester (Preparation 3, 1.23 g, 3.72 mmol) and K₂CO₃ (990 mg, 7.10 mmol) in DMF (5 mL) under argon and heated to 80° C. for 12 h. The solvent was removed in vacuo and the residue was partitioned between H₂O (75 mL) and EtOAc (75 mL). The organic phase was washed with saturated aqueous NaHCO₃ solution (75 mL) and brine (75 mL), before being dried (MgSO₄), filtered and concentrated in vacuo. Purification by column chromatography (DCM-EtOAc-MeOH, 12:2:1) afforded the title compound: RT=4.18 min, m/z (ES⁺)=517.3 [M+H]⁺. Chiral HPLC confirmed that the starting material had recemized during the reaction.

### Preparation 17: tert-Butoxycarbonylamino-(4-{3-[1-(3-isopropyl-[1,2,4]oxadiazol-5-yl)-piperidin-4-yl]propoxylphenyl)acetic acid

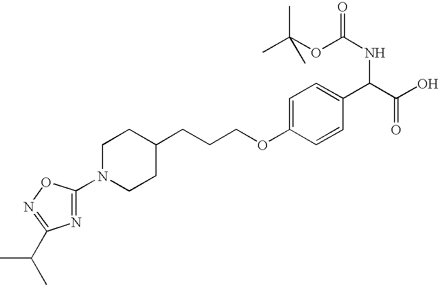

tert-Butoxycarbonylamino-(4-{3-[1-(3-isopropyl-[1,2,4]oxadiazol-5-yl)piperidin-4-yl]-propoxy}phenyl)acetic acid methyl ester (Preparation 16, 600 mg, 1.16 mmol) was dissolved in MeOH (30 mL) and H₂O (30 mL). K₂CO₃ (481 mg, 3.48 mmol) was added and the mixture was stirred under argon for 16 h before removal of the MeOH in vacuo. The aqueous layer was washed with Et₂O (50 mL), acidified to pH 3.5 by the addition of citric acid and extracted with EtOAc (2×50 mL). The combined organic extracts were dried (MgSO₄), filtered and concentrated in vacuo to afford the title compound: RT=3.85 min, m/z (ES⁺)=503.3 [M+H]⁺.

### Preparation 18: [2-((S)-2-Carbamoylpyrrolidin-1-yl)-1-(4-{3-[1-(3-isopropyl-[1,2,4]oxadiazol-5-yl)piperidin-4-yl]propoxylphenyl)-2-oxoethyl]carbamic acid tert-butyl ester

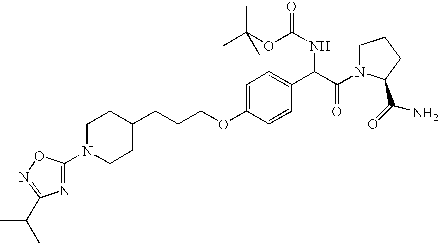

tert-Butoxycarbonylamino-(4-{3-[1-(3-isopropyl-[1,2,4]oxadiazol-5-yl)piperidin-4-yl]-propoxy}phenyl)acetic acid (Preparation 17, 520 mg, 1.03 mmol) was added to a solution of HATU (490 mg, 1.29 mmol) and DIPEA (1 mL) in DCM and the resulting solution was stirred at ambient temperature for 30 min. (S)-Proline-2-carboxamide (120 mg, 1.05 mmol) was added and the reaction was stirred at ambient temperature for 12 h. The solvent was removed in vacuo and the residue was partitioned between H₂O (50 mL) and EtOAC (50 mL). The organic phase was washed with saturated aqueous Na₂CO₃ solution (50 mL), before being dried (MgSO₄), filtered and concentrated in vacuo. Purification by RP-HPLC afforded the title compound: RT=3.67 min, m/z (ES⁺)=599.4 [M+H]⁺.

### Preparation 19: [2-((S)-2-Cyanopyrrolidin-1-yl)-1-(4-{3-[1-(3-isopropyl-[1,2,4]oxadiazol-5-yl)piperidin-4-yl]propoxylphenyl)-2-oxo-ethyl]carbamic acid tert-butyl ester

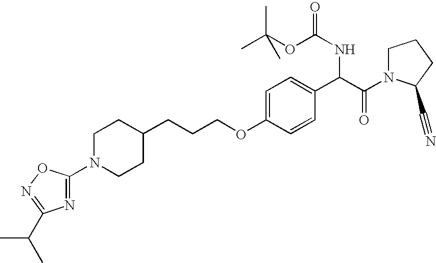

[2-((S)-2-Carbamoylpyrrolidin-1-yl)-1-(4-{3-[1-(3-isopropyl-[1,2,4]oxadiazol-5-yl)-piperidin-4-yl]propoxylphenyl)-2-oxoethyl]carbamic acid tert-butyl ester (Preparation 18, 360 mg, 600 μmol) was dissolved in anhydrous THF (15 mL) at 0° C. Trifluoroacetic anhydride (834 μL, 6.00 mmol) was added dropwise and the reaction stirred for 45 min, before diluting with DCM (50 mL) and washing with saturated aqueous NaHCO₃ solution (50 mL). The organic phase was dried (MgSO₄), filtered and concentrated in vacuo. Purification by RP-HPLC afforded the title compound. RT=4.05 min, m/z (ES⁺)=581.3 [M+H]⁺.

### Preparation 20: [(S)-2-((S)-2-Cyanopyrrolidin-1-yl)-1-(4-{3-[1-(3-isopropyl-[1,2,4]oxadiazol-5-yl)piperidin-4-yl]propoxy}phenyl)-2-oxoethyl]carbamic acid tert-butyl ester

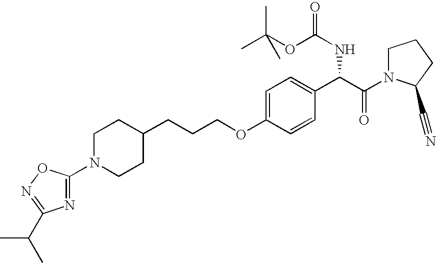

The title compound was isolated from [2-((S)-2-cyanopyrrolidin-1-yl)-1-(4-{3-[1-(3-isopropyl-[1,2,4]oxadiazol-5-yl)piperidin-4-yl]propoxylphenyl)-2-oxoethyl]carbamic acid tert-butyl ester (Preparation 19) by chiral-HPLC: RT=4.05 min, m/z (ES⁺)=581.3 [M+H]⁺ (diastereomeric excess >98%).

### Preparation 21: [(R)-2-((S)-2-Cyanopyrrolidin-1-yl)-1-(4-{3-[1-(3-isopropyl-[1,2,4]oxadiazol-5-yl)piperidin-4-yl]propoxylphenyl)-2-oxoethyl]carbamic acid tert-butyl ester

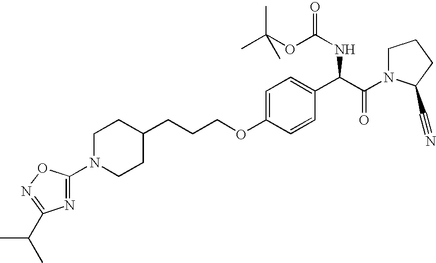

The title compound was isolated from [2-(S)-2-cyanopyrrolidin-1-yl)-1-(4-{3-[1-(3-isopropyl-[1,2,4]oxadiazol-5-yl)piperidin-4-yl]propoxy}phenyl)-2-oxoethyl]carbamic acid tert-butyl ester (Preparation 19) by chiral-HPLC: RT=4.05 min, m/z (ES⁺)=581.3 [M+H]⁺ (diastereomeric excess >98%).

### Preparation 22: 2-tert-Butoxycarbonylamino-3-(4-{(R)-3-[1-(3-isopropyl-[1,2,4]oxadiazol-5-yl)piperidin-4-yl]butoxy}phenyl)propionic acid methyl ester

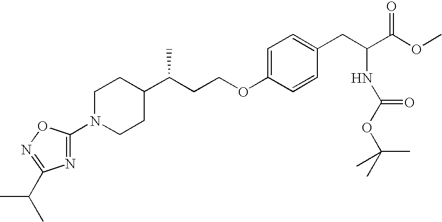

The title compound was synthesized from methanesulfonic acid (R)-3-[1-(3-isopropyl-[1,2,4]oxadiazol-5-yl)piperidin-4-yl]butyl ester (Preparation 9, 117 mg, 340 μmol) and 2-tert-butoxycarbonylamino-3-(4-hydroxyphenyl)propionic acid methyl ester (120 mg, 408 μmol) employing a procedure similar to that outlined in Preparation 16: RT=4.30 min, m/z (ES⁺)=545.2 [M+H]⁺.

### Preparation 23: 2-tert-Butoxycarbonylamino-3-(4-{(R)-3-[1-(3-isopropyl-[1,2,4]oxadiazol-5-yl)piperidin-4-yl]butoxy}phenyl)propionic acid

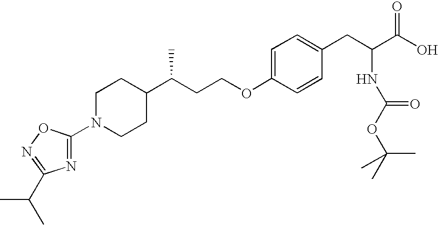

A mixture of LiOH.H₂O (12.0 mg, 600 μmol) and 2-tert-butoxycarbonylamino-3-(4-{(R)-3-[1-(3-isopropyl-[1,2,4]oxadiazol-5-yl)piperidin-4-yl]butoxy]phenyl)propionic acid methyl ester (Preparation 22, 160 mg, 300 μmol) in THF (4 mL) and H₂O (2 mL) was stirred at ambient temperature for 16 h. The THF was removed under reduced pressure, then the remainder was diluted with H₂O (20 mL), washed with EtOAc and acidified to pH 4 with 1M citric acid, before being extracted with EtOAc (2×). The combined organic extracts were dried (MgSO₄), filtered and concentrated in vacuo to afford the title compound: RT=3.98 min; m/z (ES⁺)=531.3 [M+H]⁺.

### Preparation 24: [1-(4-{(R)-3-[1-(3-Isopropyl-[1,2,4]oxadiazol-5-yl)piperidin-4-yl]butoxy}-benzyl)-2-oxo-2-thiazolidin-3-ylethyl]carbamic acid tert-butylester

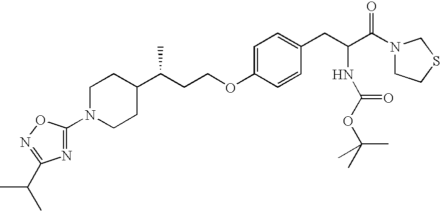

A solution of 2-tert-butoxycarbonylamino-3-(4-{(R)-3-[1-(3-isopropyl-[1,2,4]oxadiazol-5-yl)piperidin-4-yl]butoxy}phenyl)propionic acid (Preparation 23, 150 mg, 283 μmol), HOBt.H₂O (38.0 mg, 283 μmol), EDCI (54.0 mg, 283 μmol) and thiazolidine (25.0 mg, 283 μmol) in DCM (5 mL) was stirred at ambient temperature for 16 h. The reaction mixture was concentrated in vacuo and purified by column chromatography (DCM-MeOH, 1:0 to 24:1) to afford a crude material which was dissolved in EtOAc and washed with 1M citric acid (3×). The organic layer was dried (MgSO₄), filtered and concentrated in vacuo to afford the title compound: RT=4.12 min; m/z (ES⁺)=602.3 [M+H]⁺.

### Preparation 25: (S)-2-tert-Butoxycarbonylamino-3-(4-{(R)-3-[1-(3-isopropyl-[1,2,4]-5-yl)piperidin-4-yl]butoxy}phenyl)propionic acid methyl ester

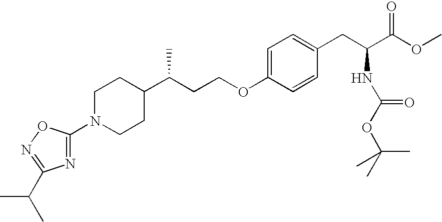

ADDP (857 mg, 34.0 mmol) in toluene (5 mL) was added to a solution of (S)-2-tert-butoxycarbonylamino-3-(4-hydroxyphenyl)propionic acid methyl ester (500 mg, 17.0 mmol), (R)-3-[1-(3-isopropyl-[1,2,4]oxadiazol-5-yl)piperidin-4-yl]butan-1-ol (Preparation 8, 454 mg, 17.0 mmol) and PBu₃ (840 μL, 34.0 mmol) in toluene (80 mL) and the resulting solution was stirred at ambient temperature for 24 h. IH (50 mL) was added to the reaction mixture and after stirring at ambient temperature for 30 min, the solid that formed was removed by filtration. The filtrate was concentrated in vacuo and purified by column chromatography (toluene-MeOH, 1:0 to 23:4) to afford the crude product. Further purification by column chromatography (EtOAc-1H, 3:7) afforded the title compound: RT=4.53 min; m/z (ES⁺)=545.2 EM+H]⁺.

### Preparation 26: (S)-2-tert-Butoxycarbonylamino-3-(4-{(R)-3-[1-(3-isopropyl-[1,2,4]oxadiazol-5-yl)piperidin-4-yl]butoxy}phenyl)propionic acid

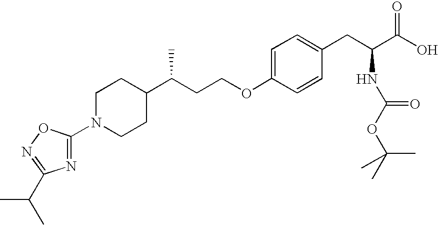

The title compound was synthesized from (S)-2-tert-butoxycarbonylamino-3-(4-{(R)-3-[1-(3-isopropyl-[1,2,4]oxadiazol-5-yl)piperidin-4-yl]butoxy}phenyl)propionic acid methyl ester (Preparation 25, 800 mg, 1.47 mmol) employing a procedure similar to that outlined in Preparation 23: RT=4.14 min; m/z (ES⁺)=531.3 [M+H]⁺.

### Preparation 27: [(S)-2-((S)-2-Carbamoylpyrrolidin-1-yl)-1-(4-{(R)-3-[1-(3-isopropyl-[1,2,4]oxadiazol-5-yl)piperidin-4-yl]butoxy}benzyl)-2-oxoethyl]carbamic acid tert-butyl ester

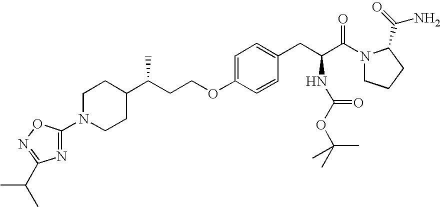

A solution of (S)-2-tert-butoxycarbonylamino-3-(4-{(R)-3-{1-(3-isopropyl-[1,2,4]oxadiazol-5-yl)piperidin-4-yl]butoxy}phenyl)propionic acid (Preparation 26, 500 mg, 943 μmol), HOBt.H₂O (143 mg, 943 μmol), EDCI (270 mg, 1.41 mmol) and (S)-pyrrolidine-2-carboxylic acid amide (160 mg, 1.41 mmol) in DCM (20 mL) was stirred at ambient temperature for 16 h. DMF (2 mL) was added to the reaction mixture to aid solubility and the resulting solution was stirred at ambient temperature for 16 h. The reaction mixture was concentrated in vacuo, then the residue was dissolved in EtOAc (45 mL) and washed with 1M NaOH (40 mL), 1M citric acid (2×50 mL) and brine (50 mL), before being dried (MgSO₄), filtered and concentrated in vacuo to afford the title compound: RT=4.12 min; m/z (ES⁺)=602.3 [M+H]⁺.

### Preparation 28: [(S)-2-((S)-2-Cyanopyrrolidin-1-yl)-1-(4-{(R)-3-[1-(3-isopropyl-[1,2,4]oxadiazol-5-yl)piperidin-4-yl]butoxy}benzyl)-2-oxoethyl]carbamic acid tert-butyl ester

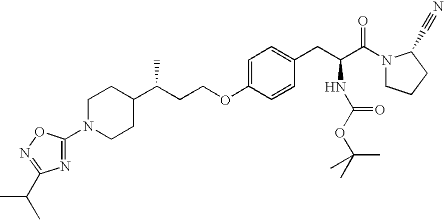

The title compound was synthesized from [(S)-2((S)-2-carbamoylpyrrolidin-1-yl)-1-(4-{(R)-3-[1-(3-isopropyl-[1,2,4]oxadiazol-5-yl)piperidin-4-yl]butoxy}benzyl)-2-oxoethyl]-carbamic acid tert-butyl ester (Preparation 27, 590 mg, 940 μmol) employing a procedure similar to that outlined in Preparation 19: RT=4.22 min; m/z (ES⁺)=609.94 [M+H]⁺.

### Preparation 29: 2-tert-Butoxycarbonylamino-3-(4-{3-[1-(3-isopropyl-[1,2,4]oxadiazol-5-yl)-piperidin-4-yl]propoxylphenyl)propionic acid methyl ester

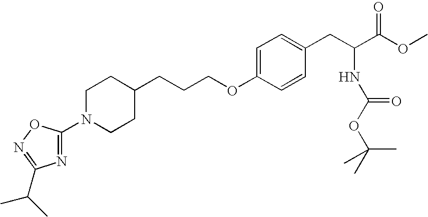

The title compound was synthesized from methanesulfonic acid 3-[1(3-isopropyl-[1,2,4]oxadiazol-5-yl)piperidin-4-yl]propyl ester (Preparation 3, 1.08 g, 3.41 mmol) and (S)-2-tert-butoxycarbonylamino-3-(4-hydroxyphenyl)propionic acid methyl ester (1.01 g, 3.41 mmol) employing a procedure similar to that outlined in Preparation 16: RT=4.24 min; m/z (ES⁺)=531.27 [M+H]⁺.

### Preparation 30: 2-tert-Butoxycarbonylamino-3-(4-{3-[1-(3-isopropyl-[1,2,4]oxadiazol-5-yl)-piperidin-4-yl]propoxylphenyl)propionic acid

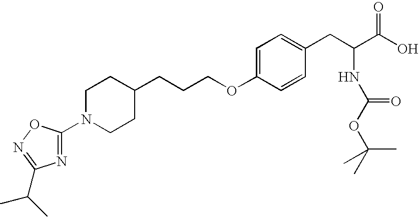

The title compound was synthesized from 2-tert-butoxycarbonylamino-3-(4-{3-[1-(3-isopropyl-[1,2,4]oxadiazol-5-yl)piperidin-4-yl]propoxylphenyl)propionic acid methyl ester (Preparation 29, 840 mg, 1.58 mmol) employing a procedure similar to that outlined in Preparation 23: RT=3.88 min; m/z (ES⁺)=517.30 [M+H]⁺.

### Preparation 31: [2-((S)-2-Carbamoylpyrrolidin-1-yl)-1-(4-{3-[1-(3-isopropyl-[1,2,4]oxadiazol-5-yl)piperidin-4-yl]propoxylbenzyl)-2-oxoethyl]carbamic acid tert-butyl ester

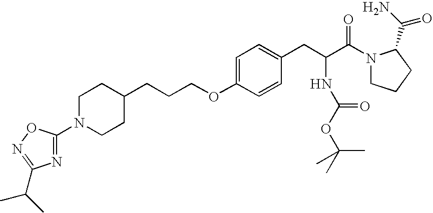

A solution of 2-tert-butoxycarbonylamino-3-(4-{3-[1-(3-isopropyl-[1,2,4]oxadiazol-5-yl)piperidin-4-yl]propoxy}phenyl)propionic acid (Preparation 30, 300 mg, 580 μmol), HATU (274 mg, 720 μmol), DIPEA (400 μL, 1.16 mmol) and (S)-pyrrolidine-2-carboxylic acid amide (82.0 mg, 720 μmol) in THF (10 mL) was stirred at ambient temperature for 20 h. The reaction mixture was diluted with DCM, washed with saturated aqueous Na₂CO₃ solution and dilute aqueous citric acid, before being dried (MgSO₄), filtered and concentrated in vacuo. Purification by column chromatography (EtOAc) afforded the title compound: RT=3.74 min, m/z (ES⁺)=613.35 [M+H]⁺.

### Preparation 32: [2-((S)-2-Cyanopyrrolidin-1-yl)-1-(4-{3-[1-(3-isopropyl-[1,2,4]oxadiazol-5-yl)piperidin-4-yl]propoxy}benzyl)-2-oxoethyl]carbamic acid tert-butyl ester

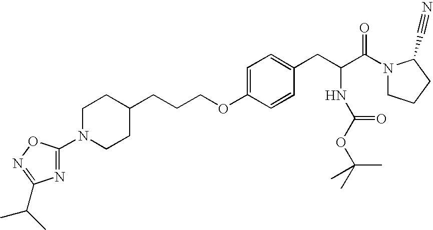

The title compound was synthesized from [2-((S)-2-carbamoylpyrrolidin-1-yl)-1-(4-{3-[1-(3-isopropyl-[1,2,4]oxadiazol-5-yl)piperidin-4-yl]propoxylbenzyl)-2-oxoethyl]carbamic acid tert-butyl ester (Preparation 31, 115 mg, 190 μmol) employing a procedure similar to that outlined in Preparation 19: RT=4.07 min, m/z (ES⁺)=595.30 [M+H]⁺.

### Preparation 33: [((S)-1-(4-{3-[1-(3-Isopropyl-[1,2,4]oxadiazol-5-yl)piperidin-4-yl]propoxy}-benzyl)-2-oxo-2-pyrrolidin-1-ylethyl]carbamic acid tert-butyl ester

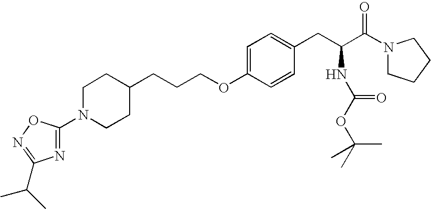

A solution of (S)-2-tert-butoxycarbonylamino-3-(4-{3-[1-(3-isopropyl-[1,2,4]oxadiazol-5-yl)piperidin-4-yl]propoxy}phenyl)propionic acid (Preparation 11, 225 mg, 436 μmol), HATU (207 mg, 545 μmol) and DIPEA (533 μL, 870 μmol) in THF (10 mL) was stirred at ambient temperature for 10 min. Pyrrolidine (73.0 μL, 870 μmol) was added and the resulting solution was stirred at ambient temperature for 3 h. The reaction mixture was diluted with DCM, washed with dilute aqueous NaOH solution, dried (MgSO₄), filtered and concentrated in vacuo.

Purification using a PE-AX column (eluting with DCM) afforded the title compound: RT=4.09 min; m/z (ES⁺)=570.39 [M+H]⁺.

### Preparation 34: 3-Fluoro-4-[(4-methoxyphenylamino)methyl]phenol

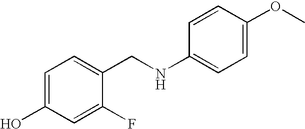

4-Methoxyphenylamine (14.0 g, 114 mol) and AcOH (6.50 mL, 114 mol) was added to a suspension of 2-fluoro-4-hydroxybenzaldehyde (5.31 g, 38.0 mol) in DCE (200 mL). After stirring at ambient temperature for 20 min, NaBH(OAc)₃ (24.1 g, 114 mol) was added in two portions and the reaction mixture was stirred at ambient temperature for 16 h. The reaction mixture was quenched with 2M NaOH, then the aqueous layer was extracted with DCM (2×100 mL) and the combined organic extracts were extracted with 2M NaOH (2×). The combined alkaline extracts were adjusted to pH 7 with 12M HCl and the resulting precipitate was collected by filtration and washed with H₂O to afford the title compound: RT=2.25 min; m/z (ES⁺)=248.09 [M+H]⁺.

### Preparation 35: [4-(tert-Butyldimethylsilanyloxy)-2-fluorobenzyl]-(4-methoxyphenyl)amine

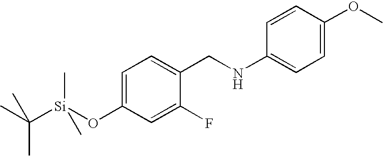

tert-Butylchlorodimethylsilane (303 mg, 2.01 mmol) was added to a solution of 3-fluoro-4-[(4-methoxyphenylamino)methyl]phenol (Preparation 34, 500 mg, 1.92 mmol) and imidazole (274 mg, 4.02 mmol) in DMF (6 mL) and the resulting solution was stirred at ambient temperature for 16 h. Further tert-butylchlorodimethylsilane was added and stirring at ambient temperature was continued for 2 h. The reaction mixture was concentrated in vacuo, then the remainder was partitioned between DCM and H₂O prior to filtration through a hydrophobic frit. The filtrate was concentrated in vacuo and purified by column chromatography (EtOAc-1H, 1:19) to afford the title compound: δ_{H} (CDCl₃) 0.21 (s, 6H), 0.98 (s, 9H), 3.75 (s, 3H), 4.26 (s, 2H), 6.54-6.66 (m, 4H), 6.76-6.82 (m, 2H), 7.17-7.24 (m, 1H).

### Preparation 36: [4-(tert-Butyldimethylsilanyloxy)-2-fluorobenzyl]-(4-methoxyphenyl)-carbamic acid tert-butyl ester

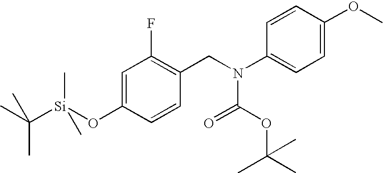

A solution of [4-(tert-butyldimethylsilanyloxy)-2-fluorobenzyl]-(4-methoxyphenyl)-amine (Preparation 35, 731 mg, 2.02 mmol), di-tert-butyl dicarbonate (464 mg, 2.10 mmol) and NEt₃ (846 μL, 6.06 mmol) in THF (35 mL) was heated at 110° C. for 3 h. The reaction mixture was concentrated in vacuo and purified by column chromatography (EtOAc-1H, 1:4) to afford the title compound: δ_{H} (CDCl₃) 0.19 (s, 6H), 0.97 (s, 9H), 1.42 (s, 9H), 3.78 (s, 3H), 4.77 (s, 2H), 6.43-6.52 (m, 1H), 6.54-6.61 (m, 1H), 6.74-6.84 (m, 2H), 6.92-7.09 (m, 2H), 7.10-7.20 (m, 1H).

### Preparation 37: (S)-[tert-Butoxycarbonyl-(4-methoxyphenyl)amino{-[4-(tert-butyldimethyl-silanyloxy)-2-fluorophenyl]acetic acid methyl ester

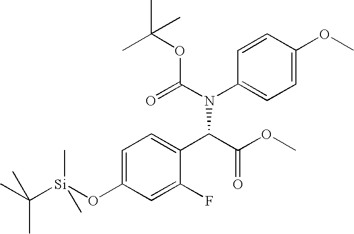

Lithium-2-butanide (1.4 M in cyclohexane, 1.12 mL, 1.56 mmol) was added to a solution of [4-(tert-butyldimethylsilanyloxy)-2-fluorobenzyl]-(4-methoxyphenyl)carbamic acid tert-butyl ester (Preparation 36, 601 mg, 1.30 mmol) and (−)-sparteine (367 mg, 1.56 mmol) in toluene (10 mL) at −78° C. The resulting solution was stirred for 1 h at −78° C. before the addition of methylchloroformate (160 mg, 1.69 mol). The reaction mixture was warmed to ambient temperature, quenched with saturated aqueous ammonium chloride solution and extracted with EtOAc (2×). The combined organic extracts were dried (MgSO₄), filtered and purified by column chromatography (EtOAc-1H, 1:9) to afford the title compound: RT=4.77 min; ink (ES⁺)=520.26 [M+H]⁺.

### Preparation 38: (S)-[tert-Butoxycarbonyl-(4-methoxyphenyl)amino{-(2-fluoro-4-hydroxy-phenyl)acetic acid methyl ester

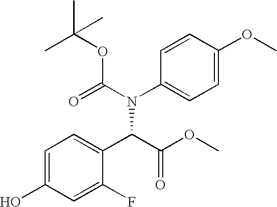

AcOH (19.0 μL, 330 μmol) and TBAF.H₂O (93.0 mg, 330 μmol) were added to a solution of (S)-[tert-butoxycarbonyl-(4-methoxyphenyl)amino{-[4-(tert-butyldimethyl-silanyloxy)-2-fluorophenyl]acetic acid methyl ester (Preparation 37, 157 mg, 300 μmol) in THF (5 mL) and the resulting solution was stirred at ambient temperature for 2 h. Saturated aqueous NaHCO₃ solution (20 mL) and EtOAc (30 mL) were added, then the aqueous layer was extracted with EtOAc (2×15 mL) and the combined organics were washed with brine, dried (MgSO₄), filtered and concentrated in vacuo. Purification by column chromatography (EtOAc-1H, 3:7) afforded the title compound: δ_{H} (CDCl₃) 1.40 (s br, 9H), 3.73 (s, 3H), 3.79 (s, 3H), 5.29 (s, 1H), 6.02 (s br, 1H), 6.35-6.48 (m, 2H), 6.66 (d, 2H), 6.82 (t, 1H), 6.92-7.07 (m, 2H).

### Preparation 39: [tert-Butoxycarbonyl-(4-methoxyphenyl)amino]-(2-fluoro-4-{3-[1-(3-isopropyl-[1,2,4]oxadiazol-5-yl)piperidin-4-yl]propoxy}phenyl)acetic acid methyl ester

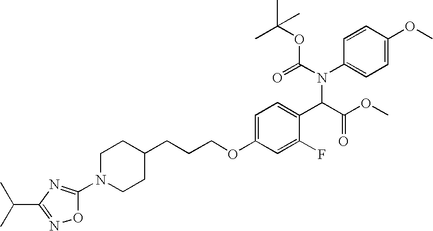

The title compound was synthesized from [tert-butoxycarbonyl-(4-methoxyphenyl)-amino]-(2-fluoro-4-{3-[1-(3-isopropyl-[1,2,4]oxadiazol-5-yl)piperidin-4-yl]propoxy}phenyl)-acetic acid methyl ester (Preparation 38, 100 mg, 250 μmol) employing a procedure similar to that outlined in Preparation 16: RT=4.49 min; m/z (ES⁺)=641.27 [M+H]⁺.

### Preparation 40: [tert-Butoxycarbonyl-(4-methoxyphenyl)amino[-(2-fluoro-4-{3-[1-(3-isopropyl-[1,2,4]oxadiazol-5-yl)piperidin-4-yl]propoxylphenyl)acetic acid

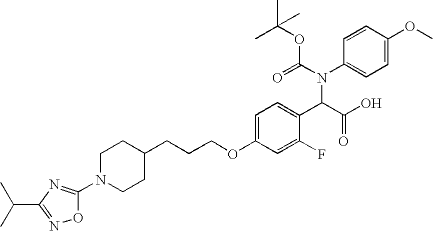

LiOH.H₂O (49.0 mg, 1.17 mmol) was added to a solution of [tert-butoxycarbonyl-(4-methoxyphenyl)amino]-(2-fluoro-4-{3-[1-(3-isopropyl-[1,2,4]oxadiazol-5-yl)piperidin-4-yl]-propoxyl}phenyl)acetic acid methyl ester (Preparation 39, 250 mg, 390 μmol) in THF (20 mL) and H₂O (2 mL) and the resulting reaction mixture was heated at 50° C. for 2 h. Further LiOH.H₂O (49.0 mg, 1.17 mmol) was added and heating at 60° C. was continued for 12 h. AcOH (134 μL, 2.34 mmol), EtOAc (80 mL) and H₂O (30 mL) were added to the reaction mixture and the resulting mixture was stirred vigorously for 1 h. The aqueous layer was extracted with EtOAc (4×20 mL) and the combined organics were washed with brine (30 mL), dried (MgSO₄), filtered and concentrated in vacuo to afford the title compound: RT=4.12 min; m/z (ES⁺)=627.24 [M+H]⁺.

### Preparation 41: [1-(2-Fluoro-4-{3-[1-(3-isopropyl-[1,2,4]oxadiazol-5-yl)piperidin-4-yl]-propoxy}phenyl)-2-oxo-2-pyrrolidin-1-ylethyl]-(4-methoxyphenyl)carbamic acid tert-butyl ester

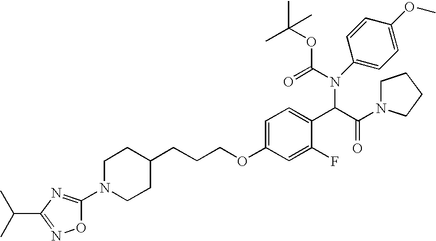

A solution of [tert-butoxycarbonyl-(4-methoxyphenyl)amino]-(2-fluoro-4-{3-[1-(3-isopropyl-[1,2,4]oxadiazol-5-yl)piperidin-4-yl]propoxy}phenyl)acetic acid (Preparation 40, 250 mg, 399 μmol), HOBt.H₂O (67.0 mg, 440 μmol) and EDCI (92.0 mg, 479 μmol) in DMF (4 mL) was stirred at ambient temperature for 5 min, followed by the addition of pyrrolidine (40.0 μL, 479 μmol), and stirring at ambient temperature was continued for 24 h. The reaction mixture was concentrated in vacuo and the remainder was partitioned between EtOAc (40 mL) and H₂O (20 mL). The aqueous layer was extracted with EtOAc (3×30 mL) and the combined organics were washed with brine, dried (MgSO₄), filtered and concentrated in vacuo. Purification by column chromatography (EtOAc-1H, 3:7) afforded the title compound: RT=4.40 min; m/z (ES⁺)=680.27 [M+H]⁺.

### Preparation 42: 2-(2-Fluoro-4-{3-[1-(3-isopropyl-[1,2,4]oxadiazol-5-yl)piperidin-4-yl]-propoxy}phenyl)-2-(4-methoxyphenylamino)-1-pyrrolidin-1-ylethanone

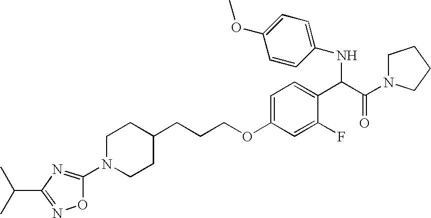

TFA (1 mL) was added to a solution of {1-(2-fluoro-4-{3-[1-(3-isopropyl-[1,2,4]oxadiazol-5-yl)piperidin-4-yl]propoxy}phenyl)-2-oxo-2-pyrrolidin-1-ylethyl]-(4-methoxyphenyl)carbamic acid tert-butyl ester (Preparation 41, 30.0 mg, 44.1 μmol) in DCM (3 mL) and the resulting solution was stirred at ambient temperature for 25 min. The solvent was removed in vacuo, then the residue was dissolved in DCM (30 mL), washed with saturated aqueous NaHCO₃ solution (2×20 mL), filtered through a hydrophobic frit and concentrated in vacuo to afford the title compound: RT=4.09 min; m/z (ES⁺)=580.26 [M+H]⁺.

### Preparation 43: (Z)-2-Acetylamino-3-(2-fluoro-4-methoxyphenyl)acrylic acid

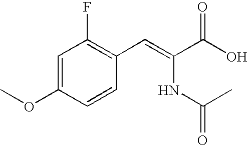

Sodium acetate (20.22 g, 246.6 mmol) and acetylaminoacetic acid (9.12 g, 77.9 mmol) were added to a solution of 2-fluoro-4-methoxybenzaldehyde (10.0 g, 64.9 mmol) in acetic anhydride (90 mL) and the resulting solution was heated at 120° C. for 5 h. The reaction mixture was added to H₂O (600 mL), extracted with EtOAc (3×200 mL) and the combined organic extracts were washed with brine, dried (MgSO₄), filtered and concentrated in vacuo. The resulting solid was suspended in dioxane (50 mL) and 0.25M HCl (400 mL) and heated at 106° C. for 1.5 h, prior to filtration of the hot reaction mixture. The filtrate was cooled to 0° C. and the resulting precipitate was collected by filtration and washed with EtOAc:MeOH (9:1). The precipitate was dissolved in THF (600 mL), washed with 1M HCl and brine, dried (MgSO₄), filtered and concentrated in vacuo to afford the title compound: δ_{H} (DMSO-d6) 1.96 (s, 3H), 3.79 (s, 3H), 6.77-6.93 (m, 2H), 7.21 (s, 1H), 7.65 (t, 1H), 9.40 (s, 1H).

### Preparation 44: 2-Acetylamino-3-(2-fluoro-4-methoxyphenyl)propionic acid

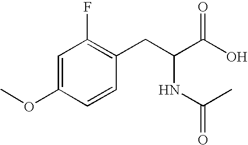

A suspension of (Z)-2-acetylamino-3-(2-fluoro-4-methoxyphenyl)acrylic acid (Preparation 43, 3.70 g, 14.6 mmol) and 10% palladium on carbon (180 mg) in EtOH (200 mL) was stirred under an atmosphere of hydrogen (30 bar) at 50° C. for 16 h. The reaction mixture was filtered and the filtrate was concentrated in vacuo to afford the title compound: δ_{H} (DMSO-d6) 1.76 (s, 3H), 2.65-2.78 (m, 1H), 3.00-3.12 (m, 1H), 3.73 (s, 3H), 4.24-4.38 (m, 1H), 6.62-6.80 (m, 2H), 7.12-7.24 (m, 1H), 7.86-7.99 (m, 1H).

### Preparation 45: 2-Amino-3-(2-fluoro-4-hydroxyphenyl)propionic acid

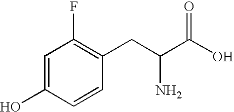

2-Acetylamino-3-(2-fluoro-4-methoxyphenyl)propionic acid (Preparation 44, 5.10 g, 19.98 mmol) was suspended in 3M HCl (200 mL) and stirred at 100° C. for 6 h. The reaction mixture was cooled to ambient temperature, filtered and the filtrate concentrated in vacuo to afford 2-amino-3-(2-fluoro-4-methoxyphenyl)propionic acid: RT=1.82 min; m/z (ES⁺)=214.00 [M+H]⁺. This product was dissolved in 48% aqueous HBr (60 mL) and stirred at 100° C. for 6 h, before diluting with H₂O (100 mL), filtering and concentrating the filtrate in vacuo. The remainder was dissolved in H₂O (100 mL), basified to pH 8 with dilute aqueous NH₃ solution and the resulting precipitate collected by filtration to afford the title compound: RT=0.82 min; m/z (ES⁺)=200.01 [M+H]⁺.

### Preparation 46: 2-tert-Butoxycarbonylamino-3-(2-fluoro-4-hydroxyphenyl)propionic acid

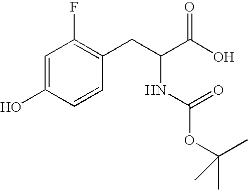

NEt₃ (2.80 mL, 20.1 mmol) and di-tert-butyl dicarbonate (3.40 g, 15.6 mmol) were added to a suspension of 2-amino-3-(2-fluoro-4-hydroxyphenyl)propionic acid (Preparation 45, 2.70 g, 13.6 mmol) in dioxane (80 mL) and H₂O (40 mL) at 0° C. The resulting suspension was stirred at 0° C. for 30 min and at ambient temperature for 16 h, before concentrating in vacuo. The remainder was dissolved in EtOAc and H₂O and acidified to pH 2 with 1M HCl. The aqueous layer was separated and extracted with EtOAc, then the combined organic extracts were washed with brine, dried (MgSO₄), filtered and concentrated in vacuo to afford the title compound: δ_{H} (DMSO-d6) 1.32 (s, 9H), 2.62-2.74 (m, 1H), 2.92-3.02 (m, 1H), 3.97-4.08 (m, 1H), 6.44-6.56 (m, 2H), 6.97-7.12 (m, 2H), 9.66 (s br, 1H).

### Preparation 47: 2-tert-Butoxycarbonylamino-3-(2-fluoro-4-hydroxyphenyl)propionic acid methyl ester

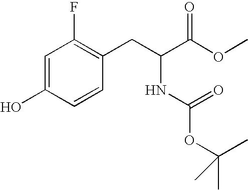

TMS-diazomethane (2M solution in hexane, 9.00 mL, 18.0 mmol) was added to a solution of 2-tert-butoxycarbonylamino-3-(2-fluoro-4-hydroxyphenyl)propionic acid (Preparation 46, 4.26 g, 13.6 mmol) in toluene:MeOH (4:1, 150 mL) at 0° C. The resulting solution was stirred at ambient temperature for 30 min, quenched with AcOH (0.5 mL) and concentrated in vacuo to afford the title compound: δ_{H} (DMSO-d6) 1.33 (s, 9H), 2.66-2.79 (m, 1H), 2.89-3.00 (m, 1H), 3.59 (s, 3H), 4.06-4.16 (m, 1H), 6.45-6.56 (m, 2H), 7.00-7.10 (m, 1H), 7.20-7.29 (m, 1H), 9.70 (s, 1H).

### Preparation 48: 2-tert-Butoxycarbonylamino-3-(2-fluoro-4-{3-[1-(3-isopropyl-[1,2,4]oxadiazol-5-yl)piperidin-4-yl]propoxy}phenyl)propionic acid methyl ester

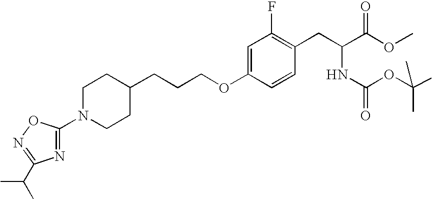

2-tert-Butoxycarbonylamino-3-(2-fluoro-4-hydroxyphenyl)propionic acid methyl ester (Preparation 47, 2.65 g, 8.46 mmol), KI (110 mg, 0.66 mmol) and K₂CO₃ (2.05 g, 14.8 mmol) were added to a solution of methanesulfonic acid 3-[1-(3-isopropyl-[1,2,4]oxadiazol-5-yl)-piperidin-4-yl]propyl ester (Preparation 3, 3.00 g, 9.10 mmol) in DMF (100 mL) and the resulting solution was stirred at 80° C. for 16 h. The reaction mixture was added to saturated aqueous NH₄Cl solution (300 mL) and H₂O (300 mL) and extracted with EtOAc (4×). The combined organic extracts were washed with H₂O and brine, dried (MgSO₄), filtered and concentrated in vacuo. Purification by column chromatography (EtOAc-1H, 1:2 to 1:1) afforded the title compound: RT=4.37 min; m/z (ES⁺)=549.31 [M+H]⁺.

### Preparation 49: 2-tert-Butoxycarbonylamino-3-(2-fluoro-4-{3-[1-(3-isopropyl-[1,2,4]oxadiazol-5-yl)piperidin-4-yl]propoxylphenyl)propionic acid

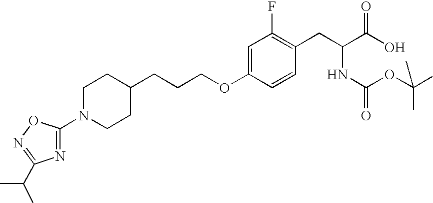

LiOH.H₂O (448 mg, 10.7 mmol) was added to a stirred solution of 2-tert-butoxycarbonylamino-3-(2-fluoro-4-{3-[1-(3-isopropyl-[1,2,4]oxadiazol-5-yl)piperidin-4-yl]-propoxy}phenyl)propionic acid methyl ester (Preparation 48, 1.95 g, 3.56 mmol) in MeOH (80 mL) and H₂O (20 mL) and the resulting solution was stirred at ambient temperature for 20 min. The MeOH was removed in vacuo, then the remainder was diluted with H₂O (100 mL), acidified to pH 4 with dilute citric acid and extracted with EtOAc (2×). The combined organics were dried (MgSO₄), filtered and concentrated in vacuo to afford the title compound: RT=3.90 min; m/z (ES⁺)=535.32 [M+H]⁺.

### Preparation 50: [S)-2-((S)-2-Carbamoylpyrrolidin-1-yl)-1-(2-fluoro-4-{3-[1-(3-isopropyl-[1,2,4]oxadiazol-5-yl)piperidin-4-yl]propoxylbenzyl)-2-oxoethyl]carbamic acid tert-butyl ester

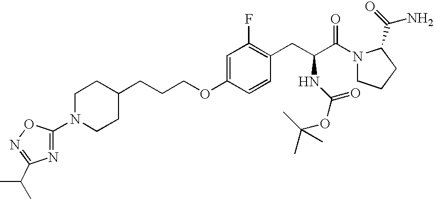

A solution of 2-tert-butoxycarbonylamino-3-(2-fluoro-4-{3-[1-(3-isopropyl-[1,2,4]oxadiazol-5-yl)piperidin-4-yl]propoxylphenyl)propionic acid (Preparation 49, 1.60 g, 3.00 mmol) and (S)-pyrrolidine-2-carboxylic acid amide (1.60 g, 3.00 mmol), HOBt.H₂O (596 mg, 3.74 mmol), EDCI (716 mg, 3.74 mmol) and DIPEA (1.05 mL, 6.00 mmol) in THF (60 mL) was stirred at ambient temperature for 15 min. (S)-pyrrolidine-2-carboxylic acid amide (427 mg, 3.74 mmol) was added and the resulting solution was stirred at ambient temperature for 16 h. The THF was removed in vacuo, then the remainder was dissolved in DCM, washed with saturated aqueous Na₂CO₃ solution and dilute citric acid, before being dried (MgSO₄), filtered and concentrated in vacuo. Recrystallization (EtOAc) afforded the unwanted stereoisomer, then the mother liquors were concentrated in vacuo and purified by column chromatography (EtOAc) to afford the title compound: RT=3.75 min; m/z (ES⁺)=631.33 [M+H]⁺.

### Preparation 51: (S)-2-((S)-2-Cyanopyrrolidin-1-yl)-1-(2-fluoro-4-{3-[1-(3-isopropyl-[1,2,4]oxadiazol-5-yl)piperidin-4-yl]propoxy}benzyl)-2-oxoethyl]carbamic acid tert-butyl ester

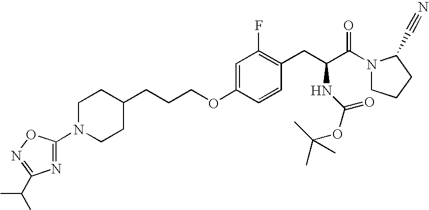

The title compound was synthesized from RS)-24(S)-2-carbamoylpyrrolidin-1-yl)-1-(2-fluoro-4-{3-[1-(3-isopropyl-[1,2,4]oxadiazol-5-yl)piperidin-4-yl]propoxy}benzyl)-2-oxoethyl]-carbamic acid tert-butyl ester (Preparation 50, 650 mg, 1.03 mmol) employing a procedure similar to that outlined in Preparation 19: RT=4.15 min; m/z (ES⁺)=613.00 [M+H]⁺.

### Preparation 52: [1-(4-{3-[1-(3-Isopropyl-[1,2,4]oxadiazol-5-yl)piperidin-4-yl]propoxy}-phenyl)-2-oxo-2-pyrrolidin-1-ylethyl]carbamic acid tert-butyl ester

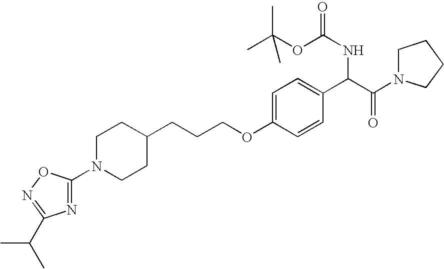

The title compound was synthesized from tert-butoxycarbonylamino-(4-{3-[1-(3-isopropyl-[1,2,4]oxadiazol-5-yl)piperidin-4-yl]propoxylphenyl)acetic acid (Preparation 17, 100 mg, 200 μmol) and pyrrolidine (16.0 mg, 220 μmol) employing a procedure similar to that outlined in Preparation 18: RT=4.18 min; m/z (ES⁺)=556.38 [M+H]⁺.

### Preparation 53: 2-tert-Butoxycarbonylamino-3-(2-fluoro-4-{(R)-3-[1-(3-isopropyl-[1,2,4]oxadiazol-5-yl)piperidin-4-yl]butoxy}phenyl)propionic acid methyl ester

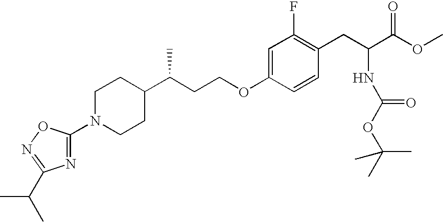

The title compound was synthesized from methanesulfonic acid (R)-3-[1-(3-isopropyl-[1,2,4]oxadiazol-5-yl)piperidin-4-yl]butyl ester (Preparation 9, 1.04 g, 3.00 mmol) and 2-tert-butoxycarbonylamino-3-(2-fluoro-4-hydroxyphenyl)propionic acid methyl ester (Preparation 47, 854 mg, 2.73 mmol) employing a procedure similar to that outlined in Preparation 16: RT=4.60 min, m/z (ES⁺)=563.3 [M+H]⁺.

### Preparation 54: 2-tert-Butoxycarbonylamino-3-(2-fluoro-4-{(R)-3-[1-(3-isopropyl-[1,2,4]oxadiazol-5-yl)piperidin-4-yl]butoxy}phenyl)propionic acid

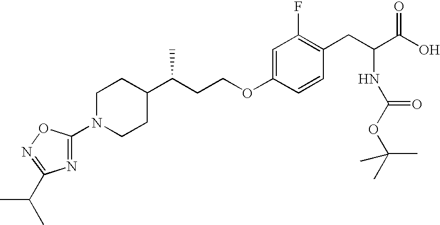

The title compound was synthesized from 2-tert-butoxycarbonylamino-3-(2-fluoro-4-{(R)-3-[1-(3-isopropyl-[1,2,4]oxadiazol-5-yl)piperidin-4-yl]butoxy}phenyl)propionic acid methylester (Preparation 53, 1.04 g, 3.00 mmol) employing a procedure similar to that outlined in Preparation 23: RT=4.18 min, m/z (ES⁺)=549.3 [M+H]⁺.

### Preparation 55: [1-(2-Fluoro-4-{(R)-3-[1-(3-isopropyl-[1,2,4]oxadiazol-5-yl)-piperidin-4-yl]-butoxy}benzyl)-2-oxo-2-thiazolidin-3-ylethyl]carbamic acid tert-butyl ester

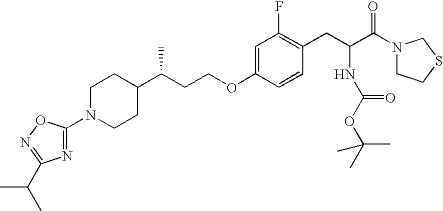

A solution of 2-tert-butoxycarbonylamino-3-(2-fluoro-4-{(R)-3-[1-(3-isopropyl-[1,2,4]oxadiazol-5-yl)piperidin-4-yl{butoxy}phenyl)propionic acid (Preparation 54, 488 mg, 891 μmol), HOBt.H₂O (121 mg, 891 μmol), EDCI (256 mg, 1.34 mmol), triethylamine (135 mg, 1.34 mmol) and thiazolidine (119 mg, 1.34 mmol) in DCM (10 mL) and DMF (3 mL) was stirred at ambient temperature for 72 h. The reaction mixture was diluted with DCM (80 mL), washed with 1M NaOH (50 mL), 1M citric acid (50 mL) and brine, then dried (MgSO₄), filtered and concentrated in vacuo. The crude product was purified by column chromatography (EtOAc-IH, 1:1) to afford the title compound: RT=4.49 min; m/z (ES⁺)=620.2 [M+H]⁺.

### Preparation 56: (S)-2-Amino-3-(2-fluoro-4-hydroxyphenyl)propionic acid hydrobromide

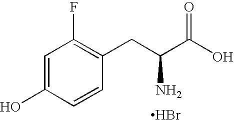

Acetic anhydride (540 g, 5.30 mol) was added under stiffing to a mixture of 2-fluoro-4-methoxybenzaldehyde (240 g, 1.56 mol), N-acetylglycine (219 g, 1.87 mol) and sodium acetate (128 g, 1.56 mol) at ambient temperature. The suspension was heated to 100° C. for 18 h. The solution was cooled to ambient temperature and the residue was alternately extracted with DCM (5×500 mL) and H₂O (5×200 mL). The remaining crystalline solid was dried to yield 4-[1-(2-fluoro-4-methoxyphenyl)meth-(E)-ylidene]-2-methyl-4H-oxazol-5-one. The DCM extracts were combined, dried (Na₂SO₄), filtered and concentrated in vacuo. The residue was recrystallized twice from EtOH to provide further 4-[1-(2-fluoro-4-methoxyphenyl)meth-(E)-ylidene{-2-methyl-4H-oxazol-5-one: RT=3.00 min (LCMS method 2).

To 4-[1-(2-fluoro-4-methoxyphenyl)meth-(E)-ylidene]-2-methyl-4H-oxazol-5-one (150.9 g, 0.642 mol) in dioxane (700 mL) was added 1 M HCl (1000 mL) and the mixture was heated under reflux conditions for 90 min. The dioxane was widely removed by evaporation and the aqueous layer was extracted with EtOAc (2×) and DCM (2×). The combined organic layers were evaporated and the residue was recrystallized from EtOAc/heptane to afford (Z)-2-acetylamino-3-(2-fluoro-4-methoxyphenyl)acrylic acid: RT=1.73 min (LCMS method 2). (Z)-2-Acetylamino-3-(2-fluoro-4-methoxyphenyl)acrylic acid (35.0 g, 138 mmol) was dissolved in MeOH (670 mL) and hydrogenated in an autoclave for 96 h with a pressure of 8 bar at 50° C. using [Rh(cod)(PP)]OTf (277 μmol) as catalyst and (S,S)-Et-Duphos as ligand (277 μmol). The solution was cooled and evaporated and the crude product was dissolved in EtOAc (550 mL). The mixture was heated to 60° C. followed by the slow addition of heptane (200 mL) before slowly cooling to ambient temperature. The solids were isolated to afford (Z)-2-acetylamino-3-(2-fluoro-4-methoxyphenyl)acrylic acid. RT=1.21 min (LCMS method 2). A tantal autoclave was charged with (Z)-2-acetylamino-3-(2-fluoro-4-methoxyphenyl)acrylic acid (71.0 g, 278 mmol), aqueous hydrobromic acid (48%, 420 mL) and acetic acid (320 mL) and heated to 105° C. for 16 h. The solvent was evaporated and the residue was subsequently triturated with Et₂O and tert-butylmethylether before being dried at 30° C. in vacuo for 3 h to afford the title compound: RT=0.815 min; m/z (ES⁺)=200 [M+H]⁺ (LCMS method 2).

### Preparation 57: (S)-2-tert-Butoxycarbonylamino-3-(2-fluoro-4-hydroxyphenyl)propionic acid

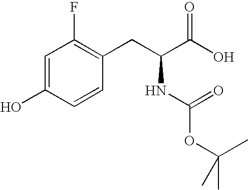

The title compound was synthesized from (S)-2-amino-3-(2-fluoro-4-hydroxyphenyl)-propionic acid hydrobromide (Preparation 56) employing a procedure similar to that outlined in Preparation 46: RT=2.82 min, m/z (ES⁺)=300.1 [M+H]⁺.

### Preparation 58: (S)-2-tert-Butoxycarbonylamino-3-(2-fluoro-4-hydroxyphenyl)propionic acid methyl ester

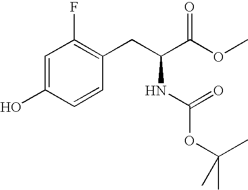

The title compound was synthesized from (S)-2-tert-butoxycarbonylamino-3-(2-fluoro-4-hydroxyphenyl)propionic acid (Preparation 57) employing a procedure similar to that outlined in Preparation 47: RT=3.18 min, m/z (ES⁺)=314.2 [M+H]⁺.

### Preparation 59: (S)-2-tert-Butoxycarbonylamino-3-(2-fluoro-4-{(R)-3-[1-(3-isopropyl-[1,2,4]oxadiazol-5-yl)piperidin-4-yl]butoxy}phenyl)propionic acid methyl ester

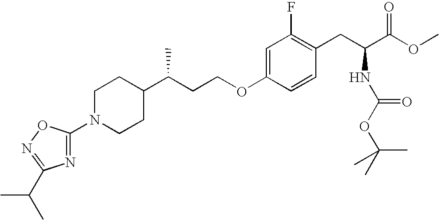

The title compound was synthesized from (R)-3-[1-(3-isopropyl-[1,2,4]oxadiazol-5-yl)piperidin-4-yl]butan-1-ol (Preparation 8, 1.30 g, 4.87 mmol) and (S)-2-tert-butoxycarbonylamino-3-(2-fluoro-4-hydroxyphenyl)propionic acid methyl ester (Preparation 58, 1.52 g, 4.87 mmol) employing a procedure similar to that outlined in Preparation 25: RT=4.35 min, m/z (ES⁺)=563.3 [M+H]⁺.

### Preparation 60: (S)-2-tert-Butoxycarbonylamino-3-(2-fluoro-4-{(R)-3-[1-(3-isopropyl-[1,2,4]oxadiazol-5-yl)piperidin-4-yl]butoxy}phenyl)propionic acid

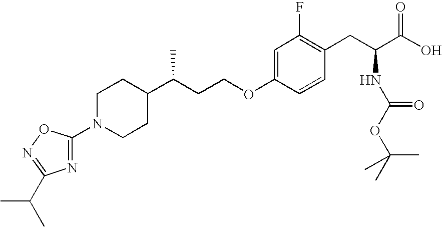

The title compound was synthesized from (S)-2-tert-butoxycarbonylamino-3-(2-fluoro-4-{(R)-3-[1-(3-isopropyl-[1,2,4]oxadiazol-5-yl)piperidin-4-yl]butoxy}phenyl)propionic acid methyl ester (Preparation 59, 1.60 g, 2.85 mmol) employing a procedure similar to that outlined in Preparation 23: RT=4.18 min, m/z (ES⁺)=549.3 [M+H]⁺.

### Preparation 61: [(S)-2-((S)-2-Carbamoylpyrrolidin-1-yl)-1-(2-fluoro-4-{(R)-3-[1-(3-isopropyl-[1,2,4]oxadiazol-5-yl)piperidin-4-yl]butoxy}benzyl)-2-oxoethyl]carbamic acid tert-butyl ester

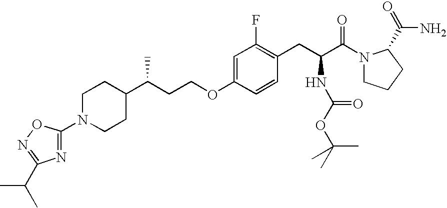

The title compound was synthesized from (S)-2-tert-butoxycarbonylamino-3-(2-fluoro-4-{(R)-3-[1-(3-isopropyl-[1,2,4]oxadiazol-5-yl)piperidin-4-yl]butoxy}phenyl)propionic acid (Preparation 60, 1.05 g, 1.92 mmol) and (S)-pyrrolidine-2-carboxylic acid amide (328 mg, 2.87 mmol) employing a procedure similar to that outlined in Preparation 55: RT=4.13 min, m/z (ES⁺)=645.3 [M+H]⁺.

### Preparation 62: [(S)-2-((S)-2-Cyanopyrrolidin-1-yl)-1-(2-fluoro-4-{(R)-3-[1-(3-isopropyl-[1,2,4]oxadiazol-5-yl)piperidin-4-yl]butoxy}benzyl)-2-oxoethyl]carbamic acid tert-butyl ester

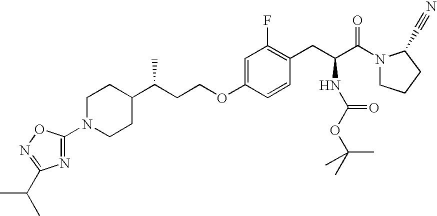

[(S)-2-((S)-2-Carbamoylpyrrolidin-1-yl)-1-(2-fluoro-4-{(R)-3-[1-(3-isopropyl-[1,2,4]oxadiazol-5-yl)piperidin-4-yl]butoxy}benzyl)-2-oxoethyl]carbamic acid tert-butyl ester (Preparation 61, 1.00 g, 1.55 mmol) was dissolved in anhydrous THF (40 mL) at 0° C. Pyridine (250 μL, 3.11 mmol) followed by trifluoroacetic anhydride (1.08 mL, 7.76 mmol) were added and the reaction was stirred for 45 min, before diluting with DCM (200 mL) and washing with saturated aqueous NaHCO₃ solution (2×200 mL), 1M citric acid (2×200 mL) and brine. The organic phase was dried (MgSO₄), filtered and concentrated in vacuo. Purification by column chromatography (EtOAc-IH, 1:1) afforded the title compound: RT=4.49 min; m/z (ES⁺)=627.3 [M+H]⁺.

### Preparation 63: [(S)-2-(3,3-Difluoroazetidin-1-yl)-1-(2-fluoro-4-{(R)-3-[1-(3-isopropyl-[1,2,4]oxadiazol-5-yl)piperidin-4-yl]butoxy}benzyl)-2-oxoethyl]carbamic acid tert-butyl ester

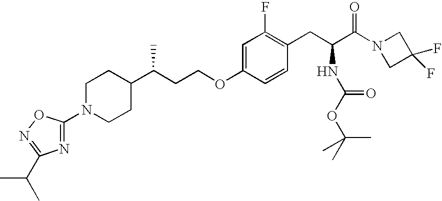

The title compound was synthesized from (S)-2-tert-butoxycarbonylamino-3-(2-fluoro-4-{(R)-3-[1-(3-isopropyl-[1,2,4]oxadiazol-5-yl)piperidin-4-yl]butoxy}phenyl)propionic acid (Preparation 60, 280 mg, 511 μmol) and 3,3-difluoroazetidine (71.0 mg, 613 μmol) employing a procedure similar to that outlined in Preparation 55: RT=4.32 min, m/z (ES⁺)=624.2 [M+H]⁺.

### Preparation 64: [(S)-2-(3,3-Difluoropyrrolidin-1-yl)-1-(2-fluoro-4-{(R)-3-[1-(3-isopropyl-[1,2,4]oxadiazol-5-yl)piperidin-4-yl]butoxy}benzyl)-2-oxoethyl]carbamic acid tert-butyl ester

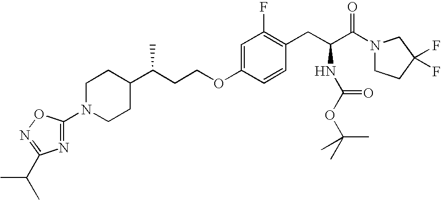

The title compound was synthesized from (S)-2-tert-butoxycarbonylamino-3-(2-fluoro-4-{(R)-3-[1-(3-isopropyl-[1,2,4]oxadiazol-5-yl)piperidin-4-yl]butoxy}phenyl)propionic acid (Preparation 60, 280 mg, 511 μmol) and 3,3-difluoropyrrolidine (79.0 mg, 613 μmol) employing a procedure similar to that outlined in Preparation 55: RT=4.35 min, m/z (ES⁺)=638.2 [M+H]⁺.

### Preparation 65: (S)-7-Hydroxy-3,4-dihydro-1H-isoquinoline-2,3-dicarboxylic acid 2-tert-butyl ester 3-methyl ester

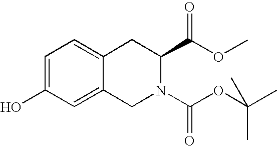

Di-(tert-butyl) dicarbonate (218 mg, 5.70 mmol) and triethylamine (770 μL, 5.40 mmol) were added to a solution of (S)-7-hydroxy-1,2,3,4-tetrahydroisoquinoline-3-carboxylic acid (1.00 g, 5.18 mmol) in dioxane (40 mL) and H₂O (20 mL) and the resulting solution was stirred at ambient temperature overnight. The dioxane was removed in vacuo and EtOAc (150 mL) was added. The organics were washed with 1M citric acid (150 mL) and brine (100 mL), before being dried (MgSO₄) and concentrated in vacuo to afford crude (S)-7-hydroxy-3,4-dihydro-1H-isoquinoline-2,3-dicarboxylic acid 2-tert-butyl ester. This compound was dissolved in DCM (40 mL) and MeOH (10 mL) and trimethylsilyl diazomethane was added dropwise until the solution remained yellow in colour. The resulting solution was stirred at ambient temperature for 1 h before adding a few drops of acetic acid and removing the solvent in vacuo to afford the title compound: RT=3.32 min, m/z (ES⁺)=308.1 [M+H]⁺.

### Preparation 66: (S)-7-{(R)-3-[1-(3-isopropyl-[1,2,4]oxadiazol-5-yl)piperidin-4-yl]butoxy}-3,4-dihydro-1H-isoquinoline-2,3-dicarboxylic acid 2-tert-butyl ester 3-methyl ester

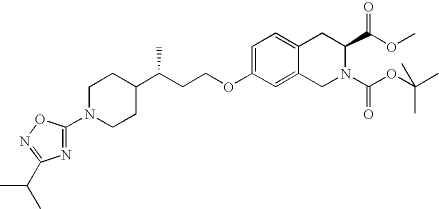

The title compound was synthesized from (S)-7-hydroxy-3,4-dihydro-1H-isoquinoline-2,3-dicarboxylic acid 2-tert-butyl ester 3-methyl ester (Preparation 65, 307 mg, 5.18 mmol) and (R)-3-[1-(3-isopropyl-[1,2,4]oxadiazol-5-yl)piperidin-4-yl]butan-1-ol (Preparation 8, 267 mg, 5.18 mmol) employing a procedure similar to that outlined in Preparation 25: RT=4.65 min, m/z (ES⁺)=557.3 [M+H]⁺.

### Preparation 67: (S)-7-{(R)-3-[1-(3-Isopropyl-[1,2,4]oxadiazol-5-yl)piperidin-4-yl]butoxy}-3,4-dihydro-1H-isoquinoline-2,3-dicarboxylic acid 2-tert-butyl ester

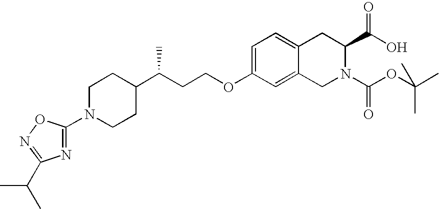

The title compound was synthesized from (S)-7-{(R)-3-[1-(3-isopropyl-[1,2,4]oxadiazol-5-yl)piperidin-4-yl]butoxyl-3,4-dihydro-1H-isoquinoline-2,3-dicarboxylic acid 2-tert-butyl ester 3-methyl ester (Preparation 66, 70.0 mg, 126 μmol) employing a procedure similar to that outlined in Preparation 23: RT=2.63 min, m/z (ES⁺)=543.4 [M+H]⁺.

### Preparation 68: (S)-3-(3,3-Difluoropyrrolidine-1-carbonyl)-7-{(R)-3-[1-(3-isopropyl-[1,2,4]oxadiazol-5-yl)piperidin-4-yl]butoxy}-3,4-dihydro-1H-isoquinoline-2-carboxylic acid text-butyl ester

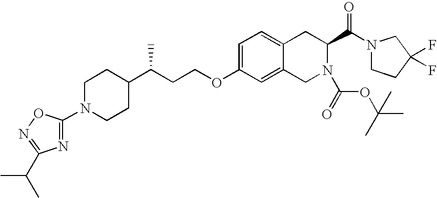

The title compound was synthesized from (S)-7-{(R)-3-[1-(3-isopropyl-[1,2,4]oxadiazol-5-yl)piperidin-4-yl]butoxy}-3,4-dihydro-1H-isoquinoline-2,3-dicarboxylic acid 2-tert-butyl ester (Preparation 67, 70.0 mg, 129 μmol) and 3,3-difluoropyrrolidine (38.0 mg, 258 μmol) employing a procedure similar to that outlined in Preparation 55: RT=4.39 min, m/z (ES⁺)=632.4 [M+H]⁺.

### Preparation 69: (S)-3-((S)-2-Carbamoylpyrrolidine-1-carbonyl)-7-{(R)-3-[1-(3-isopropyl-[1,2,4]oxadiazol-5-yl)piperidin-4-yl{butoxy}-3,4-dihydro-1H-isoquinoline-2-carboxylic acid tert-butyl ester

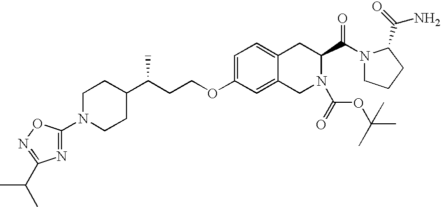

The title compound was synthesized from (S)-7-{(R)-3-[1-(3-isopropyl-[1,2,4]oxadiazol-5-yl)piperidin-4-yl]butoxy}-3,4-dihydro-1H-isoquinoline-2,3-dicarboxylic acid 2-tert-butyl ester (Preparation 67, 150 mg, 277 μmol) and (S)-pyrrolidine-2-carboxylic acid amide (62.0 mg, 415 μmol) employing a procedure similar to that outlined in Preparation 55: RT=3.87 min, m/z (ES⁺)=639.4 [M+H]⁺.

### Preparation 70: (S)-3-((S)-2-Cyanopyrrolidine-1-carbonyl)-7-{(R)-3-[1-(3-isopropyl-[1,2,4]oxadiazol-5-yl)piperidin-4-yl]butoxy}-3,4-dihydro-1H-isoquinoline-2-carboxylic acid tert-butyl ester

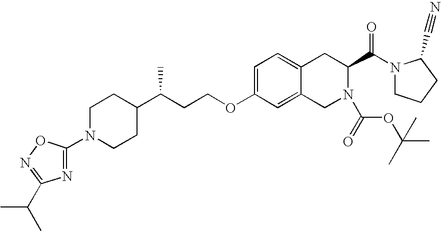

The title compound was synthesized from (S)-3-((S)-2-carbamoylpyrrolidine-1-carbonyl)-7-{(R)-3-[1-(3-isopropyl-[1,2,4]oxadiazol-5-yl)piperidin-4-yl]butoxy}-3,4-dihydro-1H-isoquinoline-2-carboxylic acid tert-butyl ester (Preparation 69, 101 mg, 315 μmol) employing a procedure similar to that outlined in Preparation 62: RT=4.36 min, m/z (ES⁺)=621.4 [M+H]⁺.

### Preparation 71: (S)-2-tert-Butoxycarbonylamino-3-(2-fluoro-4-{3-[1-(3-isopropyl-[1,2,4]oxadiazol-5-yl)piperidin-4-yl]propoxy}phenyl)propionic acid

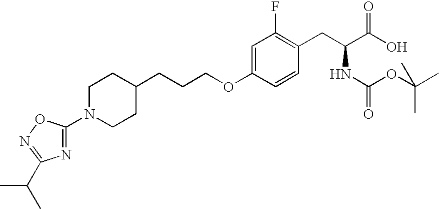

(S)-2-tert-Butoxycarbonylamino-3-(2-fluoro-4-1341-(3-isopropyl-[1,2,4]oxadiazol-5-yl)piperidin-4-yl]propoxy}phenyl)propionic acid methyl ester (408 mg, 744 μmol) was isolated from 2-tert-butoxycarbonylamino-3-(2-fluoro-4-{3-[1-(3-isopropyl-[1,2,4]oxadiazol-5-yl)-piperidin-4-yl]propoxy}phenyl)propionic acid methyl ester (Preparation 48) by chiral-HPLC and was dissolved in THF (20 mL). The solution was cooled to 0° C., LiOH.H₂O (95.0 mg, 2.26 mmol) was added and the resulting reaction mixture was stirred at 0° C. for 8 h and at ambient temperature for 16 h. The reaction mixture was acidified to pH 2-3 with 1M HCl, diluted with H₂O (300 mL) and extracted with EtOAc (4×100 mL). The combined organic extracts were washed with brine, dried (MgSO₄), filtered and concentrated in vacuo to afford the title compound: RT=4.03 min, m/z (ES⁺)=535.23 [M+H]⁺.

### Preparation 72: [(S)-2-(3,3-Difluoropyrrolidin-1-yl)-1-(2-fluoro-4-{3-[1-(3-isopropyl-[1,2,4]oxadiazol-5-yl)piperidin-4-yl]propoxy}benzyl)-2-oxoethyl]carbamic acid tert-butyl ester

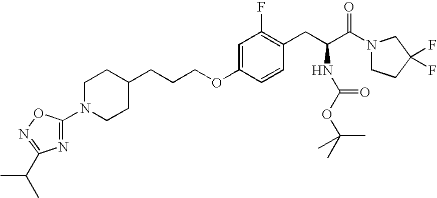

EDCI (59.3 mg, 309 μmol), HOBt (41.7 mg, 272 μmol) and 3,3-difluoropyrrolidine hydrochloride (42.7 mg, 297 μmol) were added to a solution of (S)-2-tert-butoxycarbonylamino-3-(2-fluoro-4-{3-[1-(3-isopropyl-[1,2,4]oxadiazol-5-yl)piperidin-4-yl]propoxy}phenyl)-propionic acid (Preparation 71, 133 mg, 248 μmol) in DMF (10 mL) and DIPEA (140 μL, 804 μmol) and the resulting solution was stirred at ambient temperature for 40 h. The reaction mixture was diluted with EtOAc, washed with H₂O and brine, dried (MgSO₄), filtered and concentrated in vacuo. Purification by column chromatography (EtOAc-IH, 2:1) afforded the title compound: RT=4.43 min; m/z (ES⁺)=624.3 [M+H]⁺.

### Preparation 73: [(S)-1-(2-Fluoro-4-{3-[1-(3-isopropyl-[1,2,4]oxadiazol-5-yl)piperidin-4-yl]-propoxy}benzyl)-2-((S)-3-fluoropyrrolidin-1-yl)-2-oxoethyl]carbamic acid tert-butyl ester

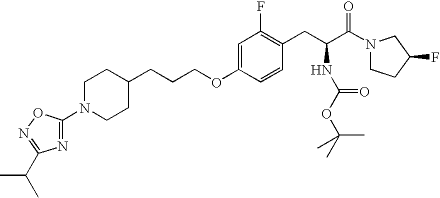

The title compound was synthesized from (S)-2-tert-butoxycarbonylamino-3-(2-fluoro-4-{3-[1-(3-isopropyl-[1,2,4]oxadiazol-5-yl)piperidin-4-yl]propoxy}phenyl)propionic acid (Preparation 71, 133 mg, 248 μmol) and (S)-3-fluoropyrrolidine hydrochloride (37.0 mg, 295 μmol) employing a procedure similar to that outlined in Preparation 72: RT=4.33 min; m/z (ES⁺)=606.32 [M+H]⁺.

### Preparation 74: (E)-3-(4-Benzyloxy-2-fluorophenyl)acrylic acid methyl ester

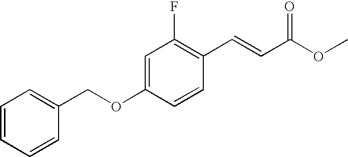

(Triphenyl-lambda*5*-phosphanylidene)acetic acid methyl ester (25.0 g, 74.8 mmol) was added to a solution of 4-benzyloxy-2-fluorobenzaldehyde (9.10 g, 39.5 mmol) in THF (400 mL) and the resulting solution was stirred under reflux conditions for 16 h, before being absorbed onto silica and purified by column chromotogarphy (EtOAc-IH, 1:3) to afford the title compound: RT=4.15 min; m/z (ES⁺)=287.17 [M+H]⁺.

### Preparation 75: (E)-3-(4-Benzyloxy-2-fluorophenyl)acrylic acid

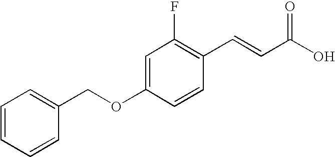

1M NaOH (40 mL, 39.8 mmol) was added to a solution of (E)-3-(4-benzyloxy-2-fluoro-phenyl)acrylic acid methyl ester (Preparation 74, 9.50 g, 33.2 mmol) in MeOH (300 mL) and the resulting suspension was stirred at ambient temperature before heating under reflux conditions for 1 h to afford a solution. The solvent was removed in vacuo and the residue was dissolved in EtOAc (300 mL) and H₂O (600 mL) before adding 1M HCl (50 mL) and stirring at ambient temperature for 30 min. The aqueous layer was separated and further extracted with EtOAc (2×). The combined organic layers were washed with brine, dried (MgSO₄), filtered and concentrated in vacuo to afford the title compound: RT=3.72 min; m/z (ES⁺)=562.31 [2M+NH₄]⁺.

### Preparation 76: (R)-3-[(E)-3-(4-Benzyloxy-2-fluorophenyl)acryloyl]-4-phenyloxazolidin-2-one

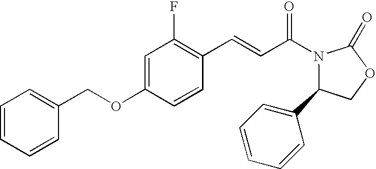

Triethylamine (4.60 mL, 33.0 mmol) and pivaloyl chloride (3.60 mL, 28.2 mmol) were added to a solution of (E)-3-(4-benzyloxy-2-fluorophenyl)acrylic acid (Preparation 75, 6.20 g, 22.8 mmol) in THF (200 mL) at −78° C. and stirred at this temperature for 15 min before stirring at 0° C. for 1 h. In a separate reaction flask, n-butyllithium (1.6M in hexane, 20 mL, 32.0 mmol) was added to a solution of R-(−)-4-phenyl-2-oxazolidinone (5.00 g, 30.6 mmol) in THF (200 mL) at −78° C. and stirred at this temperature for 20 min before adding the above solution, cooled to −78° C., via cannula. The resulting reaction mixture was stirred at −78° C. for 1.5 h and then at ambient temperature for 16 h. The reaction mixture was added to concentrated aqueous NH₄Cl solution (300 mL), then the aqueous layer was separated and further extracted with EtOAc (2×200 mL). The combined organic layers were washed with brine, dried (MgSO₄), filtered and concentrated in vacuo. Recrystallisation (EtOAc-IH, 1:1, 250 mL) afforded the title compound: RT=4.08 min; m/z (ES⁺)=418.24 [M+H]⁺.

### Preparation 77: (R)-3-[(R)-3-(4-Benzyloxy-2-fluorophenyl)butyryl]-4-phenyloxazolidin-2-one

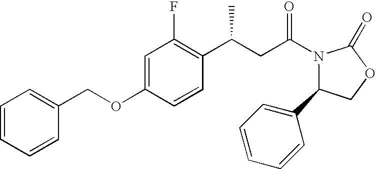

Dimethyl sulfide (30 mL) and methyl magnesium bromide (3.0 M solution in Et₂O, 13.0 mL, 39.0 mmol) were added to a suspension of copper(I) bromidedimethyl sulfide (8.80 g, 42.9 mmol) in THF (60 mL) at −40° C. and the resulting reaction mixture was stirred at this temperature for 30 min before warming to −20° C. to −15° C. A solution of (R)-3-[(E)-3-(4-benzyloxy-2-fluorophenyl)acryloyl]-4-phenyloxazolidin-2-one (Preparation 76, 40.0 g, 9.58 mmol) in THF (40 mL) was added dropwise maintaining the temperature between −25° C. and −15° C. and the solution was stirred at this temperature for 2.5 h, then at ambient temperature for 72 h. The reaction was quenched with concentrated aqueous NH₄Cl solution (50 mL) and filtered through celite. The filtrate was diluted with EtOAc, washed with H₂O and brine, dried (MgSO₄), filtered and concentrated in vacuo. Purification by column chromatography (EtOAc-IH, 1:3 to 1:2) afforded the title compound: RT=4.35 min; ink (ES⁺)=434.28 [M+H]⁺.

### Preparation 78: (R)-3-[(2R, 3S)-3-(4-Benzyloxy-5-bromo-2-fluorophenyl)-2-bromobutyryl]-4-phenyloxazolidin-2-one

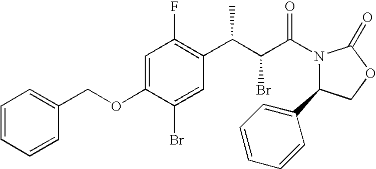

Dibutylborontriflate (10.0 mL, 10.0 mmol) and DIPEA (1.80 mL, 10.3 mmol) were added to a solution of (R)-3-[(R)-3-(4-benzyloxy-2-fluorophenyl)butyryl]-4-phenyloxazolidin-2-one (Preparation 77, 3.00 g, 6.92 mmol) in DCM (50 mL) at −78° C. The resulting reaction mixture was stirred at −78° C. for 2 h and at 0° C. for 2 h, before being quenched with 0.5 M aqueous NaHCO₃ solution. The DCM was removed in vacuo, EtOAc was added and the organic layer was washed with H₂O, brine, dried (MgSO₄), filtered and concentrated in vacuo. Purification by column chromatography (EtOAc-IH, 1:3) afforded the title compound: δ_{H} (CDCl₃) 1.52 (d, 3H), 3.63 (dd, 1H), 4.20 (dd, 1H), 4.59 (dd, 1H), 5.13 (s, 2H), 5.26 (dd, 1H), 6.12 (d, 1H), 6.68 (d, 1H), 7.30-7.51 (m, 11H).

### Preparation 79: (R)-3-[(2S,3S)-2-Azido-3-(4-benzyloxy-5-bromo-2-fluorophenyl)butyryl]-4-phenyloxazolidin-2-one

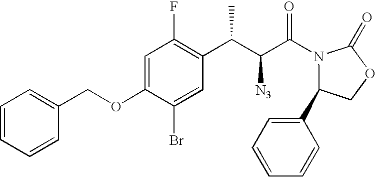

N,N,N′,N′-Tetramethylguanidinium azide (3.70 g, 23.4 mmol) was added to a solution of (R)-3-[(2R,3S)-3-(4-benzyloxy-5-bromo-2-fluorophenyl)-2-bromobutyryl]-4-phenyloxazolidin-2-one (Preparation 78, 3.40 g, 5.75 mmol) in MeCN (25 mL) and the resulting solution was stirred at ambient temperature for 16 h. The reaction mixture was diluted with EtOAc and washed with H₂O and brine, dried (MgSO₄), filtered and concentrated in vacuo. Purification by column chromatography (EtOAc-IH, 1:2) afforded the title compound: RT=4.58 min; m/z (ES⁺)=570.08, 572.08 [M+NH₄]⁺.

### Preparation 80: (2S,3S)-2-Azido-3-(4-benzyloxy-5-bromo-2-fluorophenyl)butyric acid

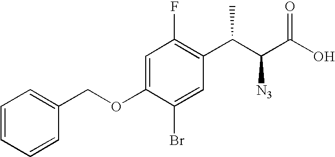

Hydrogen peroxide (35% aqueous solution, 5.00 mL) and LiOH.H₂O (810 mg, 19.3 mmol) were added to a solution of (R)-3-[(2S,3S)-2-azido-3-(4-benzyloxy-5-bromo-2-fluorophenyl)butyryl]-4-phenyloxazolidin-2-one (Preparation 79, 3.02 g, 5.46 mmol) in (THF:H₂O, 3:1) (100 mL) at 0° C. and the resulting solution was stirred at this temperature for 6 h. The reaction was quenched with 10% aqueous (w/v) Na₂SO₃ solution and stirred at ambient temperature for 1 h before quenching with H₂O (250 mL) and extracting with EtOAc (4×200 mL). The combined organics were washed with 0.5M HCl and brine, dried (MgSO₄), filtered and concentrated in vacuo to afford the title compound: RT=4.03 min; m/z (ES⁺)=425.02, 427.02 [M+NH₄]⁺.

### Preparation 81: (2S,3S)-3-(2-Fluoro-4-hydroxyphenyl)-2-methylbutyric acid hydrochloride

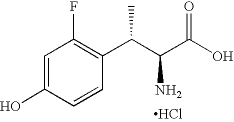

10% Palladium on carbon (1.65 g) was added to a solution of (2S,3S)-2-azido-3-(4-benzyloxy-5-bromo-2-fluorophenyl)butyric acid (Preparation 80, 2.23 g, 5.46 mmol) in (EtOH: H₂O, 9:1) (200 mL) and the resulting reaction mixture was stirred under an atmosphere of hydrogen for 72 h, before filtering through celite. The filtrate was concentrated in vacuo and the remainder dissolved in H₂O and 1M HCl, washed with EtOAc and concentrated in vacuo to afford the title compound: RT=1.71 min; m/z (ES⁺)=214.04 [M+H]⁺.

### Preparation 82: (2S,3S)-2-tert-Butoxycarbonylamino-3-(2-fluoro-4-hydroxyphenyl)butyric acid methyl ester

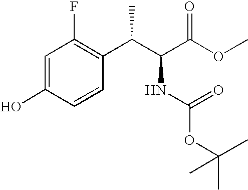

Triethylamine (320 μL, 2.30 mmol) and di-tert-butyldicarboante (500 mg, 2.30 mmol) were added to a solution of (2S,3S)-3-(2-fluoro-4-hydroxyphenyl)-2-methylbutyric acid hydrochloride (Preparation 81, 379 mg, 1.52 mmol) in (dioxane:H₂O, 10:1) (20 mL) at 0° C. and the resulting solution was warmed to ambient temperature and stirred for 16 h. The solvent was removed in vacuo and the remainder was dissolved in (EtOAc:H₂O, 3:1) (400 mL), made acidic with 1M HCl and stirred vigorously. The aqueous layer was further extracted with EtOAc (2×100 mL) and the combined organic layers were washed with brine, dried (MgSO₄), filtered and concentrated in vacuo. The remainder was dissolved in (toluene:MeOH, 4:1) (20 mL) and cooled to 0° C. before the addition of trimethylsilyl-diazomethane (2M in hexane, 1.00 mL, 2.00 mmol). The resulting reaction mixture was stirred from 0° C. to ambient temperature over 30 min, then quenched with AcOH (0.5 mL) and concentrated in vacuo. Purification by column chromatography (EtOAc-IH, 1:2) afforded the title compound: RT=3.33 min; m/z (ES⁺)=350.13 [M+Na].

### Preparation 83: (2S,3S)-2-tert-Butoxycarbonylamino-3-(2-fluoro-4-{(R)-3-[1-(3-isopropyl-[1,2,4]oxadiazol-5-yl)piperidin-4-yl]butoxy}phenyl)butyric acid methyl ester

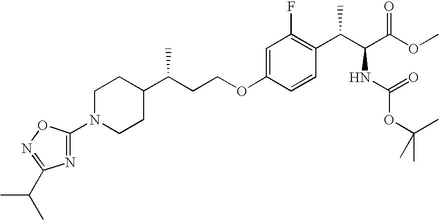

The title compound was synthesised from (2S,3S)-2-tert-butoxycarbonylamino-3-(2-fluoro-4-hydroxyphenyl)butyric acid methyl ester (Preparation 82, 480 mg, 1.47 mmol) and (R)-3-[1-(3-isopropyl-[1,2,4]oxadiazol-5-yl)piperidin-4-yl]butan-1-ol (Preparation 8, 602 mg, 2.25 mmol) employing a procedure similar to that outlined in Preparation 25: RT=4.50 min, m/z (ES⁺)=577.36 [M+H]⁺.

### Preparation 84: (2S,3S)-2-tert-Butoxycarbonylamino-3-(2-fluoro-4-{(R)-3-[1-(3-isopropyl-[[1,2,4]oxadiazol-5-yl)piperidin-4-yl]butoxy}phenyl)butyric acid

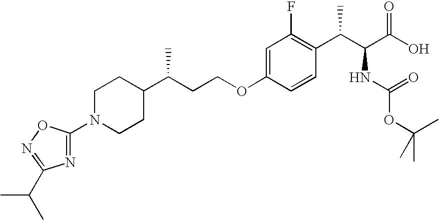

The title compound was synthesized from (2S,3S)-2-tert-butoxycarbonylamino-3-(2-fluoro-4-{(R)-3-[1-(3-isopropyl-[1,2,4]oxadiazol-5-yl)piperidin-4-yl]butoxy}phenyl)butyric acid methyl ester (Preparation 83, 525 mg, 910 μmol) employing a procedure similar to that outlined in Preparation 23: RT=4.16 min, m/z (ES⁺)=563.27 [M+H]⁺.

### Preparation 85: [(1S,2S)-1-(3,3-Difluoropyrrolidine-1-carbonyl)-2-(2-fluoro-4-{(R)-3-[1-(3-isopropyl-[1,2,4]oxadiazol-5-yl)piperidin-4-yl]butoxy}phenyl)propyl]carbamic acid tert-butyl ester

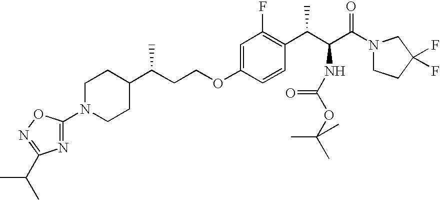

The title compound was synthesized from (2S,3S)-2-tert-butoxycarbonylamino-3-(2-fluoro-4-{(R)-3-[1-(3-isopropyl-[1,2,4]oxadiazol-5-yl)-piperidin-4-yl]-butoxy}-phenyl acid (Preparation 84, 240 mg, 426 μmol) employing a procedure similar to that outlined in Preparation 72: RT=4.44 min, m/z (ES⁺)=652.25 [M+H]⁺.

### Preparation 86: [(1S,2S)-1-((S)-2-Carbamoylpyrrolidine-1-carbonyl)-2-(2-fluoro-4-{(R)-3-[1-(3-isopropyl-[1,2,4]oxadiazol-5-yl)piperidin-4-yl]butoxy}phenyl)propyl]carbamic acid tert-butyl ester

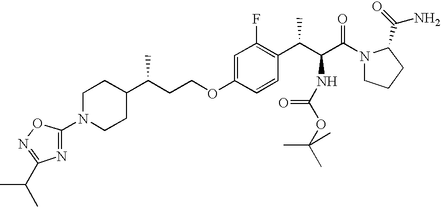

The title compound was synthesised from (2S,3S)-2-tert-butoxycarbonylamino-3-(2-fluoro-4-{(R)-3-[1-(3-isopropyl-[1,2,4]oxadiazol-5-yl)piperidin-4-yl]butoxy}phenyl)butyric acid (Preparation 84, 240 mg, 426 μmol) and (S)-pyrrolidine-2-carboxylic acid amide (102 mg, 894 μmol) employing a procedure similar to that outlined in Preparation 72: RT=3.97 min, m/z (ES⁺)=659.36 [M+H]⁺.

### Preparation 87: [(1S,2S)-1-((S)-2-Cyanopyrrolidine-1-carbonyl)-2-(2-fluoro-4-{(R)-3-[1-(3-isopropyl-[1,2,4]oxadiazol-5-yl)piperidin-4-yl]butoxy}phenyl)propyl]carbamic acid tert-butyl ester

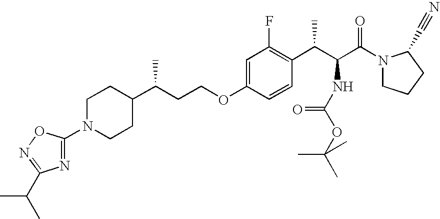

The title compound was synthesized from [(1S,2S)-1-((S)-2-carbamoylpyrrolidine-1-carbonyl)-2-(2-fluoro-4-{(R)-3-[1-(3-isopropyl-[1,2,4]oxadiazol-5-yl)piperidin-4-yl]butoxy}-phenyl)propyl]carbamic acid tert-butyl ester (Preparation 86, 218 mg, 331 μmol) employing a procedure similar to that outlined in Preparation 62: RT=4.38 min, m/z (ES⁺)=641.35 [M+H]⁺.

### Preparation 88: 4-{3-[4-((1S,2S)-2-tert-Butoxycarbonylamino-2-methoxycarbonyl-1-methyl-ethyl)-3-fluorophenoxy]propyl}piperidine-1-carboxylic acid isopropyl ester

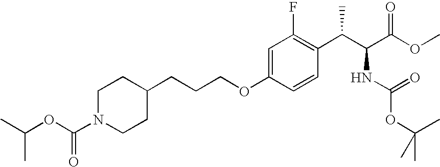

The title compound was synthesised from (2S,3S)-2-tert-butoxycarbonylamino-3-(2-fluoro-4-hydroxyphenyl)butyric acid methyl ester (Preparation 82, 148 mg, 452 μmol) and 4-(3-hydroxypropyl)piperidine-1-carboxylic acid isopropyl ester (170 mg, 741 μmol) employing a procedure similar to that outlined in Preparation 25: RT=4.40 min, m/z (ES⁺)=539.24 [M+H]⁺.

### Preparation 89: 4-{3-[4-((1S,2S)-2-tert-Butoxycarbonylamino-2-carboxy-1-methylethyl)-3-fluorophenoxy]propyl}piperidine-1-carboxylic acid isopropyl ester

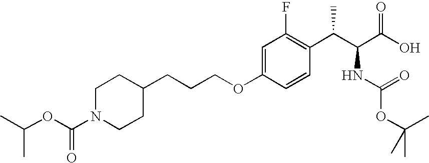

The title compound was synthesized from 4-{3-[4-((1S,2S)-2-tert-butoxycarbonylamino-2-methoxycarbonyl-1-methylethyl)-3-fluorophenoxy]propyl}piperidine-1-carboxylic acid isopropyl ester (Preparation 88, 92.0 mg, 171 μmol) employing a procedure similar to that outlined in Preparation 23: δ_{H} (CD₃OD) 1.09-1.86 (3m, 9H), 1.27 (d, 6H), 1.34 (d, 3H), 1.37 (s, 9H), 2.80 (m, 2H), 3.41 (m, 1H), 3.97 (dd, 2H),4.15 (m, 2H), 4.37 (d, 1H), 4.88 (sept, 1H), 6.65 (d, 1H), 6.71 (d, 1H), 7.20 (m, 1H).

### Preparation 90: 4-(3-{4-[(1S,2S)-2-tert-Butoxycarbonylamino-3-(3,3-difluoropyrrolidin-1-yl)-1-methyl-3-oxopropyl]-3-fluorophenoxy}propyl)piperidine-1-carboxylic acid isopropyl ester

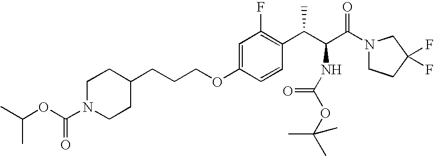

The title compound was synthesized from 4-{3-[4-((1S,2S)-2-tert-butoxycarbonylamino-2-carboxy-1-methylethyl)-3-fluorophenoxy]propyl}piperidine-1-carboxylic acid isopropyl ester (Preparation 89, 86.0 mg, 152 μmol) employing a procedure similar to that outlined in Preparation 72: RT=4.37 min, m/z (ES⁺)=614.30 [M+H]⁺.

### Preparation 91: (S)-tert-Butoxycarbonylamino-(4-{(R)-3-[1-(3-isopropyl-[1,2,4]oxadiazol-5-yl)piperidin-4-yl]butoxy}phenyl)acetic acid methyl ester

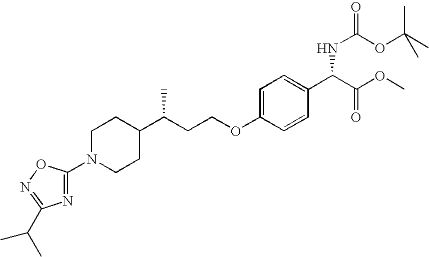

The title compound was synthesized from (R)-3-[1-(3-isopropyl-[1,2,4]oxadiazol-5-yl)piperidin-4-yl]butan-1-ol (Preparation 8, 478 mg, 1.79 mmol) and (S)-tert-butoxycarbonylamino-(4-hydroxyphenyl)acetic acid methyl ester (Preparation 15, 500 mg, 1.79 mmol) employing a procedure similar to that outlined in Preparation 25: RT=4.67 min, m/z (ES⁺)=531.26 [M+H]⁺.

### Preparation 92: (S)-tert-Butoxycarbonylamino-(4-{(R)-3-[1-(3-isopropyl-[1,2,4]oxadiazol-5-yl)piperidin-4-yl]butoxy}phenyl)acetic acid

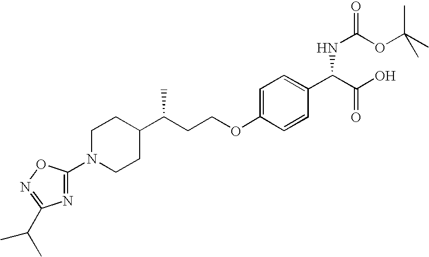

The title compound was synthesized from (S)-tert-butoxycarbonylamino-(4-{(R)-3-[1-(3-isopropyl-[1,2,4]oxadiazol-5-yl)piperidin-4-yl]butoxy}phenyl)acetic acid methyl ester (Preparation 91, 740 mg, 1.384 mmol) employing a procedure similar to that outlined in Preparation 23: RT=5.03 min, m/z (ES⁺)=517.23 [M+H]⁺.

### Preparation 93: [(S)-2-((S)-2-Carbamoylpyrrolidin-1-yl)-1-(4-{(R)-3-[1-(3-isopropyl-[1,2,4]oxadiazol-5-yl)piperidin-4-yl]butoxy}phenyl)-2-oxoethyl]carbamic acid tent-butyl ester

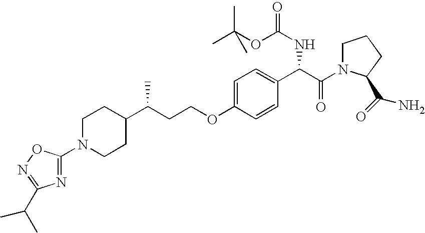

The title compound was synthesised from (S)-tert-butoxycarbonylamino-(4-{(R)-3-[1-(3-isopropyl-[1,2,4]oxadiazol-5-yl)piperidin-4-yl]butoxy}phenyl)acetic acid (Preparation 92, 300 mg, 581 μmol) and (S)-pyrrolidine-2-carboxylic acid amide (265 mg, 2.32 mmol) employing a procedure similar to that outlined in Preparation 72: RT=3.86 min, m/z (ES⁺)=613.29 [M+H]⁺.

### Preparation 94: [(S)-2-((S)-2-Cyanopyrrolidin-1-yl)-1-(4-{(R)-3-[1-(3-isopropyl-[1,2,4]oxadiazol-5-yl)piperidin-4-yl]butoxy}phenyl)-2-oxoethyl]carbamic acid tert-butyl ester

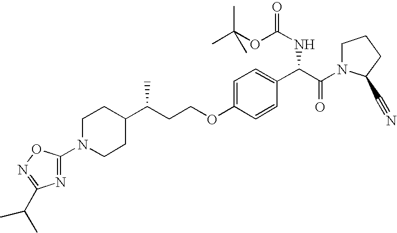

The title compound was synthesized from [(S)-2-((S)-2-carbamoylpyrrolidin-1-yl)-1-(4-{(R)-3-[1-(3-isopropyl-[1,2,4]oxadiazol-5-yl)piperidin-4-yl]butoxy}phenyl)-2-oxoethyl]-carbamic acid tert-butyl ester (Preparation 93, 350 mg, 571 μmol) employing a procedure similar to that outlined in Preparation 62: RT=4.32 min, m/z (ES⁺)=595.27 [M+H]⁺.

### Preparation 95: [(S)-2-(3,3-Difluoropyrrolidin-1-yl)-1-(4-{(R)-3-[1-(3-isopropyl-[1,2,4]oxadiazol-5-yl)piperidin-4-yl]butoxy}phenyl)-2-oxoethyl]carbamic acid tert-butyl ester

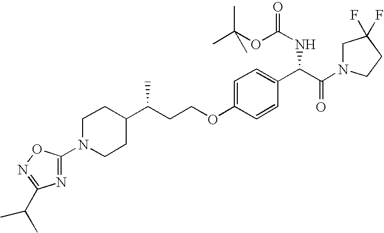

The title compound was synthesized from (S)-tert-butoxycarbonylamino-(4-{(R)-3-[1-(3-isopropyl-[1,2,4]oxadiazol-5-yl)piperidin-4-yl]butoxy}phenyl)acetic acid (Preparation 92, 180 mg, 348 μmol) employing a procedure similar to that outlined in Preparation 72: RT=4.28 min, m/z (ES⁺)=606.27 [M+H]⁺.

### Preparation 96: 4-[3-(4-Bromo-3-methylphenoxy)propyl]-1-(3-isopropyl-[1,2,4]oxadiazol-5-yl)piperidine

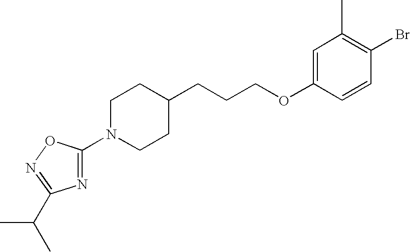

Triphenylphosphine (1.24 g, 4.74 mmol) and di-tert-butylazodicarboxylate (1.09 g, 4.74 mmol) were added to a solution of 4-bromo-3-methylphenol (886 mg, 4.74 mmol) and 3-[1-(3-isopropyl[1,2,4]oxadiazol-5-yl)piperidin-4-yl]propan-1-ol (Preparation 2, 1.00 g, 3.95 mmol) in THF (40 mL) and the resulting solution was stirred at ambient temperature for 16 h. The solvent was removed in vacuo and the residue was dissolved in EtOAc, washed with 1M NaOH, dried (MgSO₄), filtered and concentrated in vacuo. The triphenylphosphine oxide was removed by crystallization from Et₂O and the mother liquors purified by column chromatography (IH) to afford the title compound: RT=4.75 min, m/z (ES⁺)=422.10, 424.11 [M+H]⁺.

### Preparation 97: (S)-2-tert-Butoxycarbonylamino-3-(4-{3-[1-(3-isopropyl-[1,2,4]oxadiazol-5-yl)piperidin-4-yl]propoxy}-2-methylphenyl)propionic acid

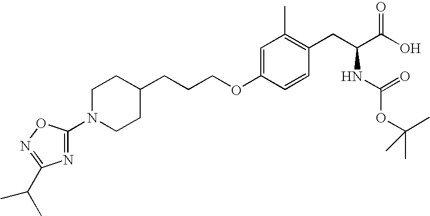

Zinc dust (207 mg, 3.16 mmol) and iodine (63.0 mg, 249 μmol) were heated under vacuum for 30 min, before cooling to 0° C. under argon and adding DMF (7 mL) and (R)-2-tert-butoxycarbonylamino-3-iodopropionic acid methyl ester (820 mg, 2.49 mmol). The resulting reaction mixture was stirred at ambient temperature for 30 min, then palladium acetate (56.0 mg, 249 μmol), PBu₃ (50.0 mg, 249 μmol) and 4-[3-(4-bromo-3-methylphenoxy)propyl]-1-(3-isopropyl-[1,2,4]oxadiazol-5-yl)piperidine (Preparation 96, 1.00 g, 2.37 mmol) were added and the resulting reaction mixture was stirred at 65° C. for 16 h. The reaction mixture was cooled to ambient temperature, diluted with EtOAc, washed with brine, dried (MgSO₄), filtered and concentrated in vacuo to afford crude (S)-2-tert-butoxycarbonylamino-3-(4-{3-[1-(3-isopropyl-[1,2,4]oxadiazol-5-yl)piperidin-4-yl]propoxy}-2-methylphenyl)propionic acid methyl ester. This crude material was suspended in MeOH (20 mL) and H₂O (5.0 mL) and cooled to 0° C. LiOH.H₂O (320 mg, 7.71 mmol) was added and the resulting reaction mixture was stirred at ambient temperature for 16 h. The solvent was removed in vacuo and the remainder was dissolved in DCM, washed with 0.1M citric acid, dried (MgSO₄), filtered and concentrated in vacuo. Purification by column chromatography afforded the title compound: RT=4.07 min, m/z (ES⁺)=531.32 [M+H]⁺.

### Preparation 98: [(S)-2-((S)-2-Carbamoylpyrrolidin-1-yl)-1-(4-{3-[1-(3-isopropyl-[1,2,4]oxadiazol-5-yl)piperidin-4-yl]propoxy}-2-methylbenzyl)-2-oxoethyl]carbamic acid tert-butyl ester

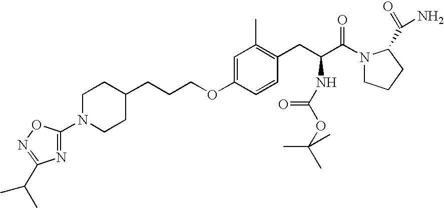

The title compound was synthesised from (S)-2-tert-butoxycarbonylamino-3-(4-{3-[1-(3-isopropyl-[1,2,4]oxadiazol-5-yl)piperidin-4-yl]propoxy}-2-methylphenyl)propionic acid (Preparation 97, 164 mg, 309 μmol) and (S)-pyrrolidine-2-carboxylic acid amide (71.0 mg, 619 μmol) employing a procedure similar to that outlined in Preparation 72: RT=3.85 min, m/z (ES⁺)=627.44 [M+H]⁺.

### Preparation 99: [(S)-2-((S)-2-Cyanopyrrolidin-1-yl)-1-(4-{3-[1-(3-isopropyl-[1,2,4]oxadiazol-5-yl)piperidin-4-yl]propoxy}-2-methylbenzyl)-2-oxoethyl]carbamic acid tert-butyl ester

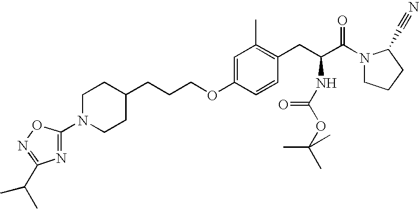

The title compound was synthesized from [(S)-2-((S)-2-carbamoylpyrrolidin-1-yl)-1-(4-{3-[1-(3-isopropyl-[1,2,4]oxadiazol-5-yl)piperidin-4-yl]propoxy}-2-methylbenzyl)-2-oxo-ethyl]carbamic acid tert-butyl ester (Preparation 98, 120 mg, 192 μmol) employing a procedure similar to that outlined in Preparation 62: RT=2.62 min, m/z (ES⁺)=609.42 [M+H]⁺.

### Preparation 100: Amino-(4-benzyloxy-2-fluorophenyl)acetonitrile

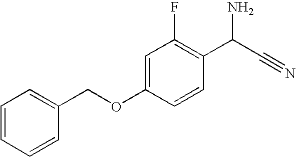

Ammonium chloride (948 mg, 17.72 mmol) was added to a solution of sodium cyanide (579 mg, 11.8 mmol) in ammonia (19 mL) and the resulting reaction mixture was cooled to 0° C. A solution of 4-benzyloxy-2-fluorobenzaldehyde (1.70 g, 7.38 mmol) in MeOH (70 mL) was added dropwise over 20 min and the reaction mixture was stirred at ambient temperature for 16 h. The solvent was removed in vacuo and the remainder was partitioned between EtOAc (100 mL) and H₂O (80 mL). The aqueous phase was extracted with EtOAc (30 mL) and the combined organics were washed with H₂O (50 mL), saturated aqueous NaHCO₃ solution (2×60 mL) and brine (60 mL), dried (MgSO₄), filtered and concentrated in vacuo to afford the title compound: δ_{H} (CDCl₃) 5.01-5.07 (m, 1H), 5.08 (s, 2H), 6.73-6.79 (m, 1H), 6.80-6.85 (m, 1H), 7.33-7.45 (m, 6H).

### Preparation 101: Amino-(2-fluoro-4-hydroxyphenyl)acetic acid hydrochloride

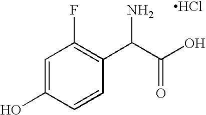

A suspension of amino-(4-benzyloxy-2-fluorophenyl)acetonitrile (Preparation 100, 2.28 g, 8.90 mmol) in 6M HCl (40 mL) was heated at 100° C. for 4 h. The solvent was removed in vacuo and the remainder was triturated with Et₂O (2×10 mL) to afford the title compound: δ_{H} (CD₃OD) 5.18 (s, 1H), 6.62-6.67 (m, 1H), 6.68-6.73 (m, 1H), 7.25-7.31 (m, 1H).

### Preparation 102: tent-Butoxycarbonylamino-(2-fluoro-4-hydroxyphenyl)acetic acid

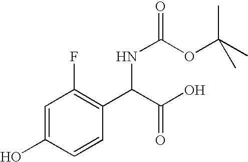

A solution of Na₂CO₃ (1.89 g, 17.8 mmol) in H₂O (25 mL) was added to a suspension of amino-(2-fluoro-4-hydroxyphenyl)acetic acid hydrochloride (Preparation 101, 2.40 g, 8.90 mmol) in dioxane (50 mL) and the resulting reaction mixture was stirred at ambient temperature for 2 min before the addition of di-tert-butyldicarbonate (2.14 g, 9.80 mmol). The reaction mixture was stirred at ambient temperature for 4 h, then EtOAc (100 mL) and 1M HCl (100 mL) were added. The aqueous phase was extracted with EtOAc and the combined organics were washed with 1M HCl (50 mL) and brine (50 mL), dried (MgSO₄), filtered and concentrated in vacuo to afford the title compound: RT=2.76 min, m/z (ES⁺)=571.2 [2M+H]⁺.

### Preparation 103: tert-Butoxycarbonylamino-(2-fluoro-4-hydroxyphenyl)acetic acid methyl ester

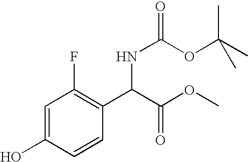

The title compound was synthesized from tert-butoxycarbonylamino-(2-fluoro-4-hydroxyphenyl)acetic acid (Preparation 102, 2.54 g, 8.90 mmol) employing a procedure similar to that outlined in Preparation 47: RT=3.29 min, m/z (ES⁺)=599.2 [2M+H]⁺.

### Preparation 104: (S)-tert-Butoxycarbonylamino-(2-fluoro-4-hydroxyphenyl)acetic acid methyl ester

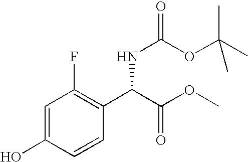

The title compound was isolated from tert-butoxycarbonylamino-(2-fluoro-4-hydroxy-phenyl)acetic acid methyl ester (Preparation 103, 2.67 g, 9.36 mmol) by chiral-HPLC: RT=3.29 min, m/z (ES⁺)=599.2 [2M+H]⁺.

### Preparation 105: (S)-tert-Butoxycarbonylamino-(2-fluoro-4-{(R)-3-[1-(3-isopropyl-[1,2,4]oxadiazol-5-yl)piperidin-4-yl]butoxy}phenyl)acetic acid methyl ester

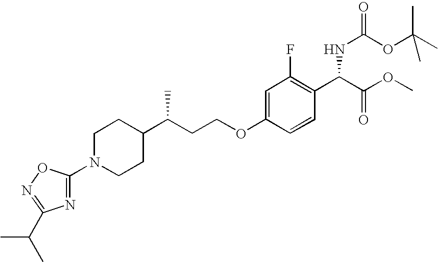

The title compound was synthesized from (R)-3-[1-(3-isopropyl-[1,2,4]oxadiazol-5-yl)piperidin-4-yl]butan-1-ol (Preparation 8, 625 mg, 2.34 mmol) and (S)-tert-butoxycarbonylamino-(2-fluoro-4-hydroxyphenyl)acetic acid methyl ester (Preparation 104, 700 mg, 2.34 mmol) employing a procedure similar to that outlined in Preparation 25: RT=4.49 min, m/z (ES⁺)=549.28 [M+H]⁺.

### Preparation 106: (S)-tert-Butoxycarbonylamino-(2-fluoro-4-{(R)-3-[1-(3-isopropyl-[1,2,4]oxadiazol-5-yl)piperidin-4-yl]butoxy}phenyl)acetic acid

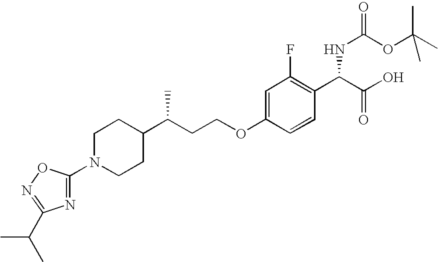

The title compound was synthesized from (S)-tert-butoxycarbonylamino-(2-fluoro-4-{(R)-3-[1-(3-isopropyl-[1,2,4]oxadiazol-5-yl)piperidin-4-yl]butoxy}phenyl)acetic acid methyl ester (Preparation 105, 780 mg, 1.42 mmol) employing a procedure similar to that outlined in Preparation 23: RT=4.05 min, m/z (ES⁺)=535.3 [M+H]⁺.

### Preparation 107: [(S)-2-(3,3-Difluoropyrrolidin-1-yl)-1-(2-fluoro-4-{(R)-3-[1-(3-isopropyl-[1,2,4]oxadiazol-5-yl)piperidin-4-yl]butoxy}phenyl)-2-oxoethyl]carbamic acid tert-butyl ester

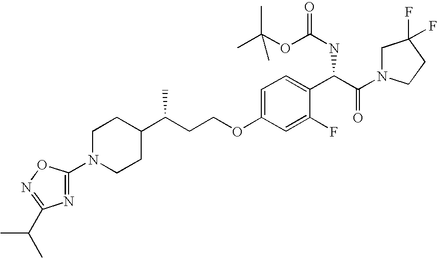

The title compound was synthesized from (S)-tert-butoxycarbonylamino-(2-fluoro-4-{(R)-3-[1-(3-isopropyl-[1,2,4]oxadiazol-5-yl)piperidin-4-yl]butoxy}phenyl)acetic acid (Preparation 106, 50.0 mg, 90.0 μmol) employing a procedure similar to that outlined in Preparation 72: RT=4.33 min, m/z (ES⁺)=624.4 [M+H]⁺.

### Preparation 108: [(S)-2-((S)-2-Carbamoylpyrrolidin-1-yl)-1-(2-fluoro-4-{(R)-3-[1-(3-isopropyl-[1,2,4]oxadiazol-5-yl)piperidin-4-yl]butoxy}phenyl)-2-oxoethyl]carbamic acid tert-butyl ester

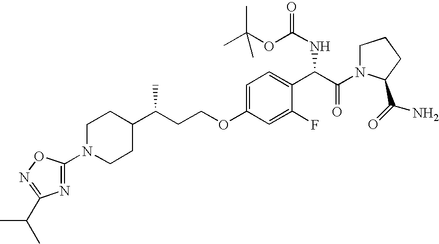

The title compound was synthesised from (S)-tert-butoxycarbonylamino-(2-fluoro-4-{(R)-3-[1-(3-isopropyl-[1,2,4]oxadiazol-5-yl)piperidin-4-yl]butoxy}phenyl)acetic acid (Preparation 106, 500 mg, 940 μmol) and (S)-pyrrolidine-2-carboxylic acid amide (214 mg, 1.87 mmol) employing a procedure similar to that outlined in Preparation 72: RT=3.91 min, m/z (ES⁺)=631.4 [M+H]⁺.

### Preparation 109: [(S)-2-((S)-2-Cyanopyrrolidin-1-yl)-1-(2-fluoro-4-{(R)-3-[1-(3-isopropyl-[1,2,4]oxadiazol-5-yl)piperidin-4-yl]butoxy}phenyl)-2-oxoethyl]carbamic acid tert-butyl ester

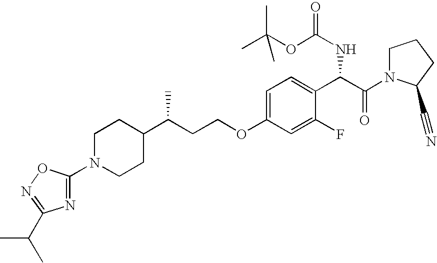

The title compound was synthesized from [(S)-2-((S)-2-carbamoylpyrrolidin-1-yl)-1-(2-fluoro-4-{(R)-3-[1-(3-isopropyl-[1,2,4]oxadiazol-5-yl)piperidin-4-yl]butoxy}phenyl)-2-oxo-ethyl]carbamic acid tert-butyl ester (Preparation 108, 360 mg, 570 μmol) employing a procedure similar to that outlined in Preparation 62: RT=4.27 min, m/z (ES⁺)=613.4 [M+H]⁺.

### Preparation 110: Amino-(4-benzyloxy-2-methylphenyl)acetonitrile

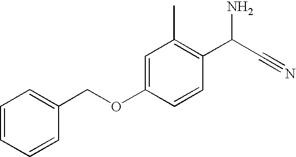

The title compound was synthesized from 2-methyl-4-benzyloxybenzaldehyde (4.00 g, 17.68 mmol) employing a procedure similar to that outlined in Preparation 100: δ_{H} (CDCl₃) 2.42 (s, 3H), 4.93-5.01 (br s, 1H), 5.08 (s, 2H), 6.83-6.89 (m, 2H), 7.30-7.52 (m, 6H).

### Preparation 111: Amino-(4-hydroxy-2-methylphenyl)acetic acid hydrochloride

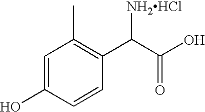

The title compound was synthesized from amino-(4-benzyloxy-2-methylphenyl)-acetonitrile (Preparation 110, 4.29 g, 17.0 mmol) employing a procedure similar to that outlined in Preparation 101: δ_{H} (DMSO) 2.33 (s, 3H), 4.94-5.03 (br s, 1H), 6.68 (s, 1H), 6.68 (s, 1H), 7.15-7.22 (m, 2H), 8.63-8.73 (br s, 2H), 9.71-9.78 (br s, 1H).

### Preparation 112: tert-Butoxycarbonylamino-(4-hydroxy-2-methylphenyl)acetic acid

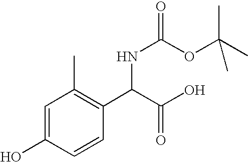

The title compound was synthesized from amino-(4-hydroxy-2-methylphenyl)acetic acid hydrochloride (Preparation 111, 3.68 g, 16.9 mmol) employing a procedure similar to that outlined in Preparation 102: RT=2.75 min, m/z (ES⁺)=282.1 [M+H]⁺.

### Preparation 113: tert-Butoxycarbonylamino-(4-hydroxy-2-methylphenyl)acetic acid methyl ester

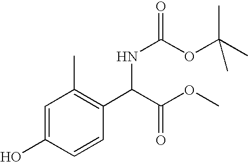

The title compound was synthesized from tert-butoxycarbonylamino-(4-hydroxy-2-methylphenyl)acetic acid (Preparation 112, 4.75 g, 16.9 mmol) employing a procedure similar to that outlined in Preparation 47: RT=3.06 min, m/z (ES⁺)=296.1 [2M+H]⁺.

### Preparation 114: tert-Butoxycarbonylamino-(4-{(R)-3-[1-(3-isopropyl-[1,2,4]oxadiazol-5-yl)-piperidin-4-yl]butoxy}-2-methylphenyl)acetic acid methyl ester

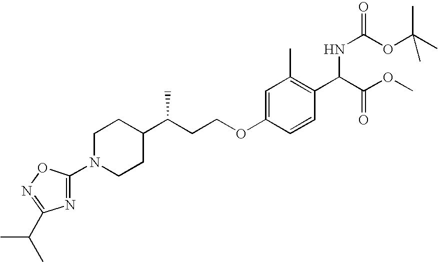

The title compound was synthesized from (R)-3-[1-(3-isopropyl-[1,2,4]oxadiazol-5-yl)piperidin-4-yl]butan-1-ol (Preparation 8, 1.99 g, 7.45 mmol) and tert-butoxycarbonylamino-(4-hydroxy-2-methylphenyl)acetic acid methyl ester (Preparation 113, 2.20 g, 7.45 mmol) employing a procedure similar to that outlined in Preparation 25: RT=4.43 min, m/z (ES⁺)=545.3 [M+H]⁺.

### Preparation 115: tert-Butoxycarbonylamino-(4-{(R)-3-[1-(3-isopropyl-[1,2,4]oxadiazol-5-yl)-piperidin-4-yl]butoxy}-2-methylphenyl)acetic acid

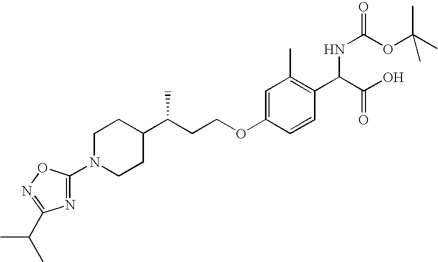

The title compound was synthesized from tert-butoxycarbonylamino-(4-{(R)-3-[1-(3-isopropyl-[1,2,4]oxadiazol-5-yl)piperidin-4-yl]butoxy}-2-methylphenyl)acetic acid methyl ester (Preparation 114, 2.39 g, 4.39 mmol) employing a procedure similar to that outlined in Preparation 23: RT=4.05 min, m/z (ES⁺)=531.2 [M+H]⁺.

### Preparation 116: [2-((S)-2-Carbamoylpyrrolidin-1-yl)-1-(4-{(R)-3-[1-(3-isopropyl-[1,2,4]oxadiazol-5-yl)piperidin-4-yl]butoxy}-2-methylphenyl)-2-oxoethy]carbamic acid tert-butyl ester

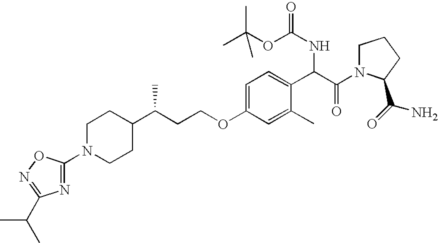

The title compound was synthesised from tert-butoxycarbonylamino-(4-{(R)-3-[1-(3-isopropyl-[1,2,4]oxadiazol-5-yl)piperidin-4-yl]butoxy}-2-methylphenyl)acetic acid (Preparation 115, 500 mg, 940 μmol) and (S)-pyrrolidine-2-carboxylic acid amide (215 mg, 1.88 mmol) employing a procedure similar to that outlined in Preparation 72: RT=3.91 min, m/z (ES⁺)=627.3 [M+H]⁺.

### Preparation 117: [2-(((S)-2-Cyanopyrrolidin-1-yl)-1-(4-{(R)-3-[1-(3-isopropyl-[1,2,4]oxadiazol-5-yl)piperidin-4-yl]butoxy}-2-methylphenyl)-2-oxoethyl]carbamic acid tert-butyl ester

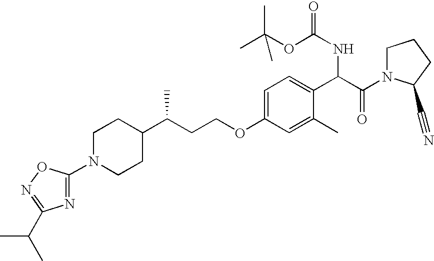

The title compound was synthesized from [2-((S)-2-carbamoylpyrrolidin-1-yl)-1-(4-{(R)-3-[1-(3-isopropyl-[1,2,4]oxadiazol-5-yl)piperidin-4-yl]butoxy}-2-methylphenyl)-2-oxo-ethy]carbamic acid tert-butyl ester (Preparation 116, 320 mg, 510 μmol) employing a procedure similar to that outlined in Preparation 62: RT=4.30 min, m/z (ES⁺)=609.4 [M+H]⁺.

### Preparation 118: [(S)-2-((S)-2-Cyanopyrrolidin-1-yl)-1-(4-{(R)-3-[1-(3-isopropyl-[1,2,4]oxadiazol-5-yl)piperidin-4-yl]butoxy}-2-methylphenyl)-2-oxoethyl]carbamic acid tert-butyl ester

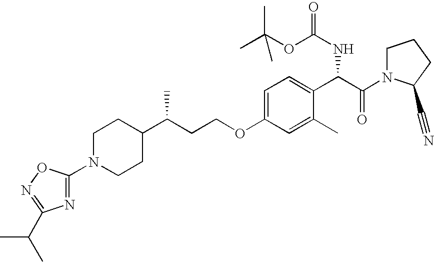

The title compound was isolated from [2-((S)-2-cyanopyrrolidin-1-yl)-1-(4-{(R)-3-[1-(3-isopropyl-[1,2,4]oxadiazol-5-yl)piperidin-4-yl]butoxy}-2-methylphenyl)-2-oxoethyl]-carbamic acid tert-butyl ester (Preparation 117, 296 mg, 486 μmol) by chiral-HPLC: RT=4.30 min, m/z (ES⁺)=609.4 [M+H]⁺.

### Preparation 119: (R)-3-[1-(5-Chloropyrimidin-2-yl)piperidin-4-yl{-butan-1-ol

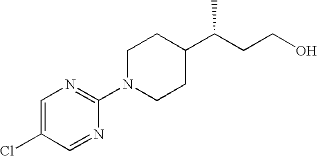

TFA (75 mL) was added to a solution of tert-butyl 4-((R)-3-hydroxy-1-methylpropyl)piperidine-1-carboxylate (Preparation 6, 30.0 g, 117 mmol) in DCM (150 mL) at 0° C. and the resulting solution was stirred at this temperature for 0.5 h. The solvent was removed in vacuo and the remainder dissolved in DCM then washed with saturated aqueous NaHCO₃ solution, dried (MgSO₄), filtered and concentrated in vacuo to afford (R)-3-piperidin-4-yl-butan-1-ol. To a portion of this material (10.0 g, 63.7 mmol) in DMSO (65 mL) was added DBU (14.3 mL, 95.5 mmol) and 2,5-dichloropyrimidine (14.3 g, 95.5 mmol) and the resulting reaction mixture was heated at 100° C. for 1.5 h. The reaction mixture was cooled to ambient temperature, quenched with H₂O and extracted with EtOAc. The organic extracts were washed with 1M HCl and brine, dried (MgSO₄), filtered and concentrated in vacuo. Purification by column chromatography (EtOAc-IH; 1:4 to 7:13) afforded the title compound: RT=3.58 min, m/z (ES⁺)=270.08 [M+H]⁺.

### Preparation 120: (R)-3-[1-(5-Chloropyrimidin-2-yl)piperidin-4-yl]butyraldehyde

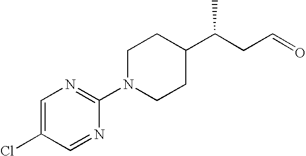

Dess-Martin Periodinane (7.50 g, 17.8 mmol) was added to a solution of (R)-3-[1-(5-chloropyrimidin-2-yl)piperidin-4-yl]butan-1-ol (Preparation 119, 4.00 g, 14.8 mmol) in DCM (100 mL) and the resulting reaction mixture stirred at ambient temperature for 16 h. Saturated aqueous NaHCO₃ solution (100 mL) and Na₂S₆O₃(5.00 g) were added and the reaction mixture was stirred for 0.5 h. The organic phase was separated and washed with H₂O and brine, dried (MgSO₄), filtered and concentrated in vacuo. Purifictaion by column chromatography (EtOAc-IH, 1:4 to 3:7) afforded the title compound: RT=3.85 min, m/z (ES⁺)=268.18 [M+H]⁺.

### Preparation 121: 5-Chloro-2-[4-((R)-1-methylbut-3-ynyl)piperidin-1-yl]pyrimidine

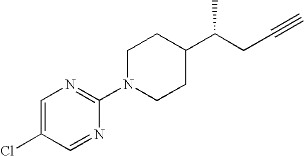

nBuLi (2.5M in hexane, 8.00 mL, 20.0 mmol) was added to a solution of diisopropylamine (3.10 mL, 22.0 mmol) in THF (8.90 mL) at −30° C. and the resulting solution was stirred at this temperature for 1 h. A portion of this solution (7.46 mL, 7.46 mmol) was transferred to a separate flask at −78° C. and trimethylsilyldiazomethane (2.0M in Et₂O, 3.73 mL, 7.47 mmol) was added. The resulting orange solution was stirred at −78° C. for 45 min before the addition of (R)-3-[1-(5-chloropyrimidin-2-yl)piperidin-4-yl]butyraldehyde (Preparation 120, 2.00 g, 7.47 mmol) in THF (40 mL). The reaction mixture was stirred at −78° C. for 1 h and then at 0° C. for 30 min before quenching with saturated aqueous ammonium chloride solution and diluting with Et₂O. The organics were separated, washed with 1M citric acid, H₂O and brine, dried (MgSO₄), filtered and concentrated in vacuo. Purification by column chromatography (EtOAc-IH, 1:9) afforded the title compound: RT=2.66 min, m/z (ES⁺)=264.06 [M+H]⁺.

### Preparation 122: (S)-2-tert-Butoxycarbonylamino-3-(2-fluoro-4-trifluoromethanesulfonyloxy-phenyl)propionic acid methyl ester

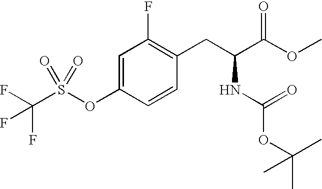

N-Phenyl-bis(trifluoromethylsulfonimide) (2.85 g, 7.98 mmol) was added to a solution of (S)-2-tert-butoxycarbonylamino-3-(2-fluoro-4-hydroxyphenyl)propionic acid methyl ester (Preparation 58, 2.00 g, 6.38 mmol) and DIPEA (1.12 mL, 7.98 mmol) in MeCN (100 mL) at 0° C. and the resulting reaction mixture was stirred at ambient temperature for 16 h. The solvent was removed in vacuo, then the remainder was dissolved in EtOAc, washed with H₂O, 1M HCl and brine and dried (MgSO₄). Removal of the solvent in vacuo and purification by column chromatography (EtOAc-IH, 1:4 to 2:3) afforded the title compound: (S_{H} (CDCl₃) 1.40 (s, 9H), 3.06 (dd, 1H), 3.24 (dd, 1H), 3.74 (s, 3H), 4.63-4.57 (m, 1H), 5.08 (d, 1H), 7.08-7.02 (m, 2H), 7.30-7.25 (m, 1H).

### Preparation 123: (S)-2-tert-Butoxycarbonylamino-3-(4-{(R)-4-[1-(5-chloropyrimidin-2-yl)-piperidin-4-yl]pent-1-ynyl}-2-fluorophenyl)propionic acid methyl ester

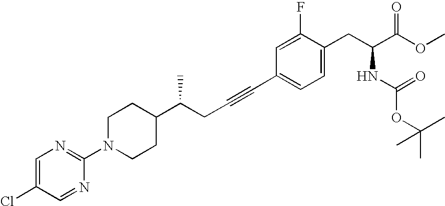

5-Chloro-2-[4-((R)-1-methylbut-3-ynyl)piperidin-1-yl]pyrimidine (Preparation 121, 622 mg, 2.36 mmol) in DMF (3 mL) was added to a solution of (S)-2-tert-butoxycarbonylamino-3-(2-fluoro-4-trifluoromethanesulfonyloxyphenyl)propionic acid methyl ester (Preparation 122, 700 mg, 2.36 mmol) and triethylamine (4.40 mL, 31.3 mmol) in DMF (10 mL) and the resulting solution was degassed with argon for 20 min. Pd(PPh₃)₄ (182 mg, 160 μmol) and CuI (60.0 mg, 310 mmol) were added and the resulting reaction mixture was stirred at 70° C. for 16 h. The solvent was removed in vacuo and the crude material was purified by column chromatography (EtOAc-IH, 1:4) to afford the title compound: RT=4.87 min, m/z (ES⁺)=559.27 [M+H]⁺.

### Preparation 124: (S)-2-tent-Butoxycarbonylamino-3-(4-{(R)-4-{1-(5-chloropyrimidin-2-yl)-piperidin-4-yl]pent-1-ynyl}-2-fluorophenyl)propionic acid

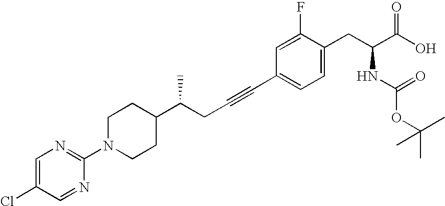

The title compound was synthesized from (S)-2-tent-butoxycarbonylamino-3-(4-{(R)-4-[1-(5-chloropyrimidin-2-yl)piperidin-4-yl]pent-1-ynyl}-2-fluorophenyl)propionic acid methyl ester (Preparation 123, 850 mg, 1.52 mmol) employing a procedure similar to that outlined in Preparation 23: RT=4.62 min, m/z (ES⁺)=545.20 [M+H]⁺.

### Preparation 125: [(S)-1-(4-{(R)-4-[1-(5-Chloropyrimidin-2-yl)piperidin-4-yl]pent-1-ynyl}-2-fluorobenzyl)-2-(3,3-difluoropyrrolidin-1-yl)-2-oxoethyl]carbamic acid tert-butyl ester

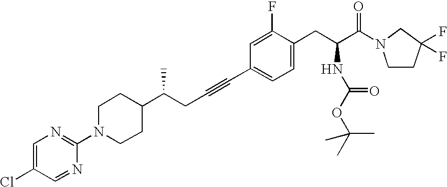

The title compound was synthesized from (S)-2-tert-butoxycarbonylamino-3-(4-{(R)-4-[1-(5-chloropyrimidin-2-yl)piperidin-4-yl]pent-1-ynyl}-2-fluorophenyl)propionic acid (Preparation 124, 828 mg, 1.52 mmol) employing a procedure similar to that outlined in Preparation 72: RT=4.85 min, m/z (ES⁺)=634.22 [M+H]⁺.

### Preparation 126: [(S)-1-(4-{(R)-4-[1-(5-Chloropyrimidin-2-yl)piperidin-4-yl]pent}-2-fluorobenzyl)-2-(3,3-difluoropyrrolidin-1-yl)-2-oxoethyl]carbamic acid tert-butyl ester

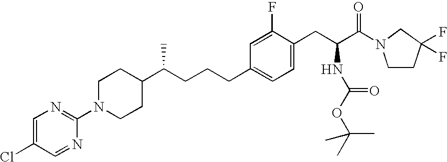

Pd(OH)₂ on carbon (10.0 mg) was added to a solution of [(S)-1-(4-{(R)-4-[1-(5-chloropyrimidin-2-yl)piperidin-4-yl]pent-1-ynyl}-2-fluorobenzyl)-2-(3,3-difluoropyrrolidin-1-yl)-2-oxoethyl]carbamic acid tert-butyl ester (Preparation 125, 170 mg, 269 μmol) in EtOH (3 mL) and the resulting reaction mixture was stirred under an atmosphere of hydrogen for 20 h at ambient temperature. The solvent was removed in vacuo and the crude material was purified by chiral-HPLC to afford the title compound: RT=2.94 min, m/z (ES⁺)=638.29 [M+H[⁺.

### Preparation 127: 4-{(R)-3-[1-(3-Isopropyl-[1,2,4]oxadiazol-5-yl)piperidin-4-yl]butoxy}-benzaldehyde

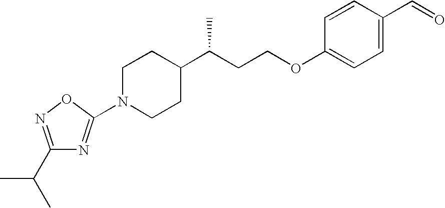

The title compound was synthesized from methanesulfonic acid (R)-3-[1-(3-isopropyl-[1,2,4[oxadiazol-5-yl)piperidin-4-yl]butyl ester (Preparation 9, 3.60 g, 10.4 mmol) and 4-hydroxybenzaldehyde (1.16 g, 9.50 mmol) employing a procedure similar to that outlined in Preparation 16: RT=4.12 min, m/z (ES⁺)=372.33 [M+H[⁺.

### Preparation 128: (S)-tert-Butoxycarbonylamino-(4-{(R)-3-[1-(5-chloropyrimidin-2-yl)-piperidin-4-yl]butoxy}phenyl)acetic acid methyl ester

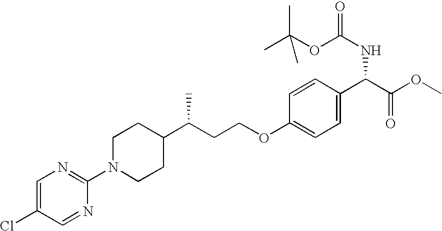

The title compound was synthesized from (R)-3-[1-(5-chloropyrimidin-2-yl)piperidin-4-yl]butan-1-ol (Preparation 119, 1.00 g, 3.70 mmol) and (S)-tert-butoxycarbonylamino-(4-hydroxyphenyl)acetic acid methyl ester (Preparation 15, 1.00 g, 3.70 mmol) employing a procedure similar to that outlined in Preparation 25: RT=5.00 min, m/z (ES⁺)=533.17 [M+H]⁺.

### Preparation 129: (S)-tert-Butoxycarbonylamino-(4-{(R)-3-[1-(5-chloropyrimidin-2-yl)-piperidin-4-yl]butoxy}phenyl)acetic acid

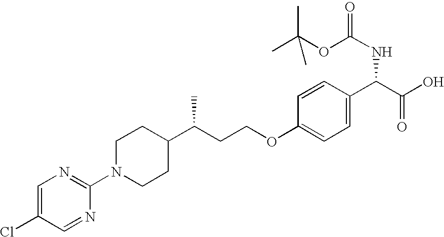

The title compound was synthesized from (S)-tert-butoxycarbonylamino-(4-{(R)-3-[1-(5-chloropyrimidin-2-yl)piperidin-4-yl]butoxy}phenyl)acetic acid methyl ester (Preparation 128, 1.30 g, 2.40 mmol) employing a procedure similar to that outlined in Preparation 23: RT=4.39 min, m/z (ES⁺)=519.14 [M+H]⁺.

### Preparation 130: [(S)-1-(4-{(R)-3-[1-(5-Chloropyrimidin-2-yl)piperidin-4-yl]butoxy}phenyl)-2-(3,3-difluoropyrrolidin-1-yl)-2-oxoethyl]carbamic acid tert-butyl ester

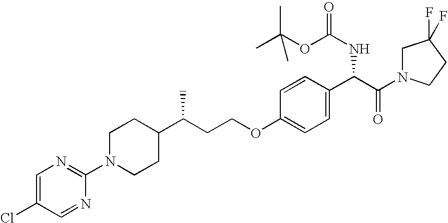

The title compound was synthesized from (S)-tert-butoxycarbonylamino-(4-{(R)-3-[1-(5-chloropyrimidin-2-yl)piperidin-4-yl]butoxy}phenyl)acetic acid (Preparation 129, 200 mg, 390 μmol) employing a procedure similar to that outlined in Preparation 72: RT=4.69 min, m/z (ES⁺)=608.30 [M+H]⁺.

### Preparation 131: (R)-3-[1-(3-Ethyl-[1,2,4]oxadiazol-5-yl)piperidin-4-yl]butan-1-ol

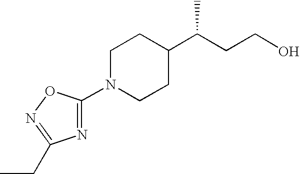

The title compound was synthesized from 4-((R)-3-hydroxy-1-methylpropyl)piperidine-1-carbonitrile (Preparation 7) and N-hydroxypropionamidine employing a procedure similar to that outlined in Preparation 2: RT=2.87 min, m/z (ES⁺)=254.13 [M+H]⁺.

### Preparation 132: (S)-tent-Butoxycarbonylamino-(4-{(R)-3-[1-(3-ethyl-[1,2,4]oxadiazol-5-yl)-piperidin-4-yl]butoxy}phenyl)acetic acid methyl ester

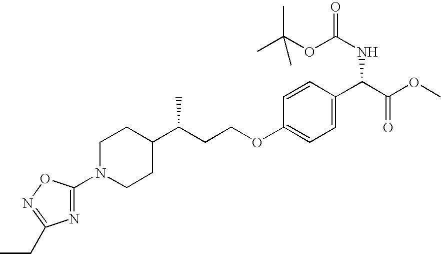

The title compound was synthesized from (R)-3-[1-(3-ethyl-[1,2,4]oxadiazol-5-yl)-piperidin-4-yl]butan-1-ol (Preparation 131, 1.57 g, 6.20 mmol) and (S)-tert-butoxycarbonylamino-(4-hydroxyphenyl)acetic acid methyl ester (Preparation 15, 2.60 g, 9.30 mmol) employing a procedure similar to that outlined in Preparation 25: RT=4.28 min, m/z (ES⁺)=517.25 [M+H]⁺.

### Preparation 133: (S)-tent-Butoxycarbonylamino-(4-{(R)-3-[1-(3-ethyl-[1,2,4]oxadiazol-5-yl)-piperidin-4-yl]butoxy}phenyl)acetic acid

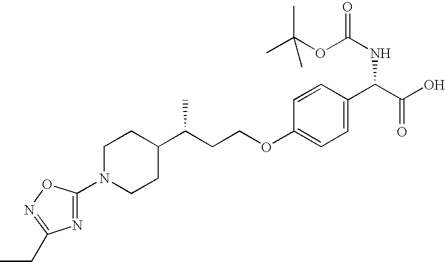

The title compound was synthesized from (S)-tert-butoxycarbonylamino-(4-{(R)-3-[1-(3-ethyl-[1,2,4]oxadiazol-5-yl)piperidin-4-yl]butoxy}phenyl)acetic acid methyl ester (Preparation 132, 1.21 g, 2.30 mmol) employing a procedure similar to that outlined in Preparation 23: RT=3.82 min, m/z (ES⁺)=503.25 [M+H]⁺.

### Preparation 134: [(S)-2-((S)-2-Carbamoylpyrrolidin-1-yl)-1-(4-{(R)-3-[1-(3-ethyl-[1,2,4]oxadiazol-5-yl)piperidin-4-yl]butoxy}phenyl)-2-oxoethyl]carbamic acid tert-butyl ester

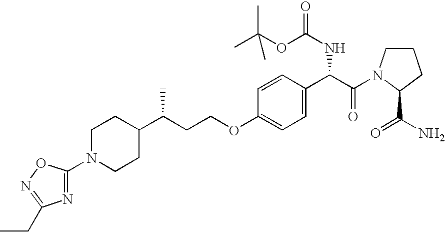

The title compound was synthesised from (S)-tert-butoxycarbonylamino-(4-{(R)-3-[1-(3-ethyl-[1,2,4]oxadiazol-5-yl)piperidin-4-yl]butoxy}phenyl)acetic acid (Preparation 133, 770 mg, 1.53 mmol) and (S)-pyrrolidine-2-carboxylic acid amide (350 mg, 3.06 mmol) employing a procedure similar to that outlined in Preparation 72: RT=3.71 min, m/z (ES⁺)=599.4 [M+H]⁺.

### Preparation 135: [(S)-2-((S)-2-Cyanopyrrolidin-1-yl)-1-(4-{(R)-3-[1-(3-ethyl-[1,2,4]oxodiazol-5-yl)piperidin-4-yl]butoxy}phenyl)-2-oxoethyl]carbamic acid tert-butyl ester

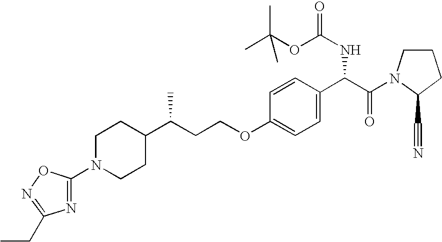

The title compound was synthesized from [(S)-2-((S)-2-carbamoylpyrrolidin-1-yl)-1-(4-{(R)-3-[1-(3-ethyl-[1,2,4]oxadiazol-5-yl)piperidin-4-yl]butoxy}phenyl)-2-oxoethyl]carbamic acid tert-butyl ester (Preparation 134, 563 mg, 940 μmol) employing a procedure similar to that outlined in Preparation 62: RT=4.05 min, m/z (ES⁺)=581.38 [M+H]⁺.

### Preparation 136: [2-((S)-2-Carbamoylpyrrolidin-1-yl)-1-(2-fluoro-4-{3-[1-(3-isopropyl-[[1,2,4]oxadiazol-5-yl)piperidin-4-yl]propoxy}phenyl)-2-oxoethyl]-(4-methoxyphenyl)carbamic acid tert-butyl ester

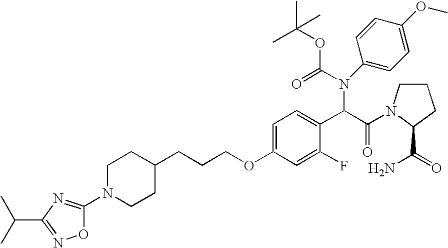

The title compound was synthesized from [tert-butoxycarbonyl-(4-methoxyphenyl)-amino]-(2-fluoro-4-{3-[1-(3-isopropyl-[1,2,4]oxadiazol-5-yl)piperidin-4-yl]propoxy}phenyl)-acetic acid (Preparation 40, 200 mg, 319 μmol) and (S)-pyrrolidine-2-carboxylic acid amide (44.0 mg, 383 μmol) employing a procedure similar to that outlined in Preparation 41: RT=4.27 min; m/z (ES⁺)=723.29 [M+H]⁺.

### Preparation 137: [2-(S)-2-Cyanopyrrolidin-1-yl)-1-(2-fluoro-4-{3-[1-(3-isopropyl-[1,2,4]oxadiazol-5-yl)piperidin-4-yl]propoxy}phenyl)-2-oxoethyl]-(4-methoxyphenyl)carbamic acid tert-butyl ester

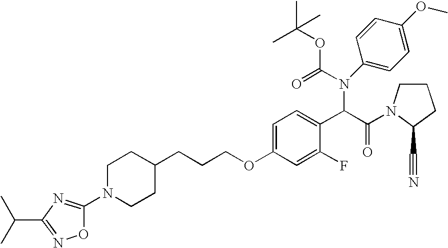

The title compound was synthesized from [2-((S)-2-carbamoylpyrrolidin-1-yl)-1-(2-fluoro-4-{3-[1-(3-isopropyl-[1,2,4]oxadiazol-5-yl)piperidin-4-yl]propoxy}phenyl)-2-oxoethyl]-(4-methoxyphenyl)carbamic acid tert-butyl ester (Preparation 136, 163 mg, 226 μmol) employing a procedure similar to that outlined in Preparation 62: RT=4.41 min; m/z (ES⁺)=705.49 [M+H]⁺.

### Preparation 138: (S)-1-[2-(2-Fluoro-4-{3-[1-(3-isopropyl-[1,2,4]oxadiazol-5-yl)piperidin-4-yl]-propoxy}phenyl)-2-(4-methoxyphenylamino)acetyl]pyrrolidine-2-carbonitrile

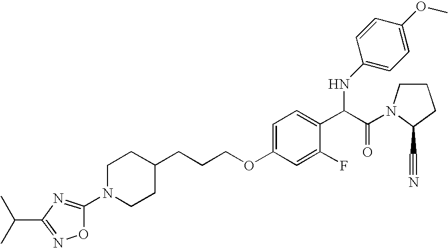

The title compound was synthesized from [2-((S)-2-cyanopyrrolidin-1-yl)-1-(2-fluoro-4-{3-[1-(3-isopropyl-[1,2,4]oxadiazol-5-yl)piperidin-4-yl]propoxy}phenyl)-2-oxoethyl]-(4-methoxyphenyl)carbamic acid tert-butyl ester (Preparation 137, 133 mg, 189 μmol) employing a procedure similar to that outlined in Preparation 42: RT=4.29 min; m/z (ES⁺)=605.28 [M+H]⁺.

### Example 1

### (S)-2-Amino-3-(4-{3-[1-(3-isopropyl-[1,2,4]oxadiazol-5-yl)piperidin-4-yl]-propoxy}phenyl)-1-thiazolidin-3-ylpropan-1-one

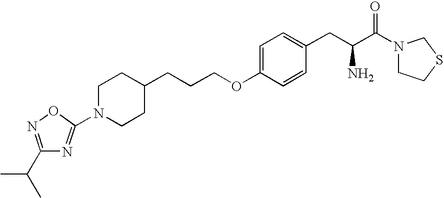

TFA (500 μL) was added to a solution of [(S)-1-(4-{3-[1-(3-isopropyl-[1,2,4]oxadiazol-5-yl)piperidin-4-yl]propoxy}benzyl)-2-oxo-2-thiazolidin-3-ylethyl]carbamic acid tert-butyl ester (Preparation 12, 60.0 mg, 100 μmol) in DCM (4.5 mL) at 0° C. and stirred at ambient temperature for 5 h. The reaction mixture was diluted with DCM (20 mL), washed with saturated aqueous Na₂CO₃ solution, dried (MgSO₄) and concentrated in vacuo. Purification by column chromatography (MeOH-DCM, 1:19) afforded the title compound: RT=2.92 min; m/z (ES⁺)=488.24 [M+H]⁺.

### Example 2

### (S)-2-Amino-1-((S)-3-fluoropyrrolidin-1-yl)-3-(4-{3-[1-(3-isopropyl-[1,2,4]oxadiazol-5-yl)piperidin-4-yl]propoxy}phenyl)propan-1-one

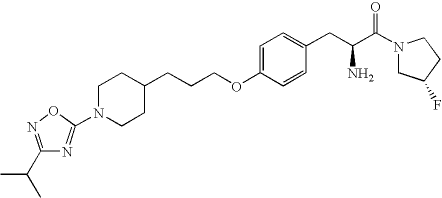

The title compound was synthesised from [(S)-2-((S)-3-fluoropyrrolidin-1-yl)-1-(4-{3-[1-(3-isopropyl-[1,2,4]oxadiazol-5-yl)piperidin-4-yl]propoxy}benzyl)-2-oxoethyl]carbamic acid tert-butyl ester (Preparation 13, 90.0 mg, 150 μmol) employing a procedure similar to that outlined in Example 1: RT=2.97 min; m/z (ES⁺)=488.00 [M+H]⁺.

### Example 3

### (S)-1-[(S)-2-Amino-2-(4-{3-[1-(3-isopropyl-[1,2,4]oxadiazol-5-yl)piperidin-4-yl]-propoxy}phenyl)acetyl]pyrrolidine-2-carbonitrile

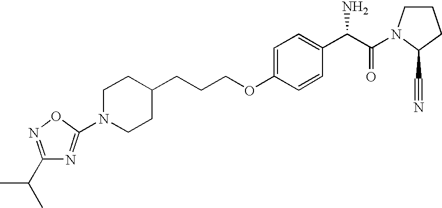

TFA (1 mL) was added to a solution of [(S)-2-((S)-2-cyanopyrrolidin-1-yl)-1-(4-{3-[1-(3-isopropyl-[1,2,4]oxadiazol-5-yl)piperidin-4-yl]propoxy}phenyl)-2-oxoethyl]carbamic acid tert-butyl ester (Preparation 20, 30.0 mg, 50.0 μmol) in DCM (5 mL) and the resulting solution was stirred for 30 min. The reaction was diluted with DCM (20 mL) and adjusted to pH 8 by the addition of saturated aqueous NaHCO₃ solution. The organic layer was dried (MgSO₄), filtered and concentrated in vacuo to afford the title compound: RT=2.90 min, m/z (ES⁺)=481.3 [M+H]⁺.

### Example 4

### (S)-1-[(R)-2-Amino-2-(4-{3-[1-(3-isopropyl-[1,2,4]oxadiazol-5-yl)piperidin-4-yl]-propoxy}phenyl)acetyl]pyrrolidine-2-carbonitrile

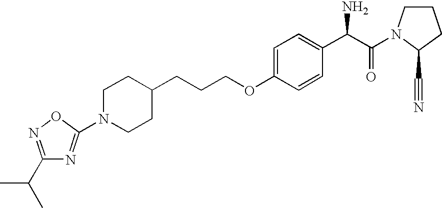

The title compound was synthesized from [(R)-2-((S)-2-cyanopyrrolidin-1-yl)-1-(4-{3-[1-(3-isopropyl-[1,2,4]oxadiazol-5-yl)piperidin-4-yl]propoxy}phenyl)-2-oxoethyl]carbamic acid tert-butyl ester (Preparation 21, 30.0 mg, 50.0 μmol) employing a procedure similar to that outlined in Example 3: RT=2.87 min, m/z (ES⁺)=481.3 [M+H]⁺.

### Example 5

### (S)-2-Amino-3-(4-{(R)-3-[1-(3-isopropyl-[1,2,4]oxadiazol-5-yl)piperidin-4-yl]-butoxy}phenyl)-1-thiazolidin-3-ylpropan-1-one

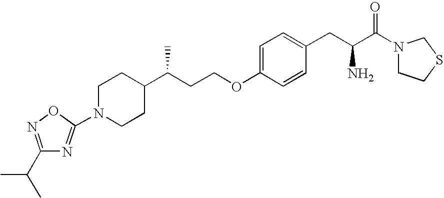

[(S)-1-(4-{(R)-3-[1-(3-Isopropyl-[1,2,4]oxadiazol-5-yl)piperidin-4-yl]butoxy}benzyl)-2-oxo-2-thiazolidin-3-ylethyl]carbamic acid tert-butyl ester was isolated from [1-(4-{(R)-3-[1-(3-isopropyl-[1,2,4]oxadiazol-5-yl)piperidin-4-yl]butoxy}benzyl)-2-oxo-2-thiazolidin-3-ylethyl]-carbamic acid tert-butylester (Preparation 24) by chiral-HPLC. To a solution of this compound (25.0 mg, 42 μmol) in DCM (2 mL) was added 4M HCl in dioxane (40 μL) and the resulting solution was stirred at ambient temperature for 1 h. Further 4M HCl in dioxane (40 μL) was added and the reaction mixture was stirred at ambient temperature for a further 72 h. The reaction mixture was concentrated in vacuo and the residue was dissolved in DCM (5 mL), before adding MP-carbonate Resin. The reaction mixture was stirred at ambient temperature for 2 h, prior to filtration and concentration of the filtrate in vacuo to afford the title compound: RT=3.08 min, m/z (ES⁺)=502.3 [M+H⁺.

### Example 6

### (S)-1-[(S)-2- Amino-3-(4-{(R)-3-[1-(3-isopropyl-[1,2,4]oxadiazol-5-yl)piperidin-4-yl]butoxy}phenyl)propionyl]pyrrolidine-2-carbonitrile

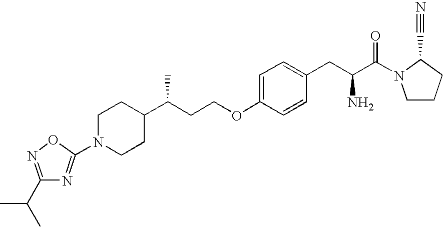

10% TFA in DCM (10 mL) was added to [(S)-2-((S)-2-cyanopyrrolidin-1-yl)-1-(4-{(R)-3-[1-(3-isopropyl-[1,2,4]oxadiazol-5-yl)piperidin-4-yl]butoxy}benzyl)-2-oxoethyl]carbamic acid tert-butyl ester (Preparation 28, 180 mg, 296 μmol) and the resulting solution was stirred at ambient temperature for 1.5 h. The reaction mixture was concentrated in vacuo, azeotroping with toluene (2×50 mL), then the residue was dissolved in DCM (10 mL) and MeOH (5 mL). MP-Carbonate was added and the resulting mixture was stirred at ambient temperature for 2 h. The resin was removed by filtration and the filtrate was concentrated in vacuo to afford the title compound: RT=2.88 min, m/z (ES⁺)=509.3 [M+H]⁺.

### Example 7

### (S)-1-[(S)-2- Amino-3-(4-{3-[1-(3-isopropyl-[1,2,4]oxadiazol-5-yl)piperidin-4-yl]-propoxy}phenyl)propionyl]pyrrolidine-2-carbonitrile

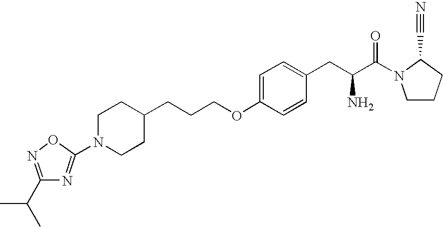

The title compound was synthesised from [2-((S)-2-cyanopyrrolidin-1-yl)-1-(4-{3-[1-(3-isopropyl-[1,2,4]oxadiazol-5-yl)piperidin-4-yl]propoxy}benzyl)-2-oxoethyl]carbamic acid tert-butyl ester (Preparation 32, 70.0 mg, 118 μmol) employing a procedure similar to that outlined in Example 1. Purification by column chromatography (MeOH-DCM, 3:97 to 1:19) afforded a single diasteroisomer: RT=2.85 min, m/z (ES⁺)=495.26 [M+H]⁺.

### Example 8

### (S)-2-Amino-3-(4-{3-[1-(3-isopropyl-[1,2,4]oxadiazol-5-yl)piperidin-4-yl]-propoxy}phenyl)-1-pyrrolidin-1-ylpropan-1-one

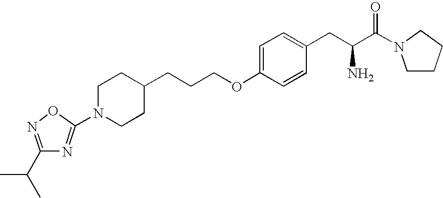

The title compound was synthesized from [(S)-1-(4-{3-[1-(3-isopropyl-[1,2,4]oxadiazol-5-yl)piperidin-4-yl]propoxy}benzyl)-2-oxo-2-pyrrolidin-1-ylethyl]carbamic acid tert-butyl ester (Preparation 33, 120 mg, 210 μmol) employing a procedure similar to that outlined in Example 1: RT=2.90 min, m/z (ES⁺)=470.50 [M+H]⁺.

### Example 9

### (S)-2-Amino-2-(2-fluoro-4-{3-[1-(3-isopropyl-[1,2,4]oxadiazol-5-yl)piperidin-4-yl]propoxy}phenyl)-1-pyrrolidin-1-ylethanone

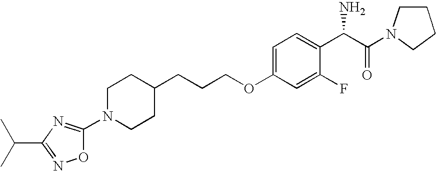

Ceric ammonium nitrate (49.0 mg, 90.0 μmol) was added to a solution of 2-(2-fluoro-4-{3-[1-(3-isopropyl-[1,2,4]oxadiazol-5-yl)piperidin-4-yl]propoxy}phenyl)-2-(4-methoxy-phenylamino)-1-pyrrolidin-1-ylethanone (Preparation 42, 26.0 mg, 45.0 μmol) in MeCN (3 mL) at 0° C., and the resulting solution was stirred at this temperature for 30 min. Saturated aqueous Na₂S₂O₃ solution was added and the mixture partitioned between EtOAc (30 mL) and H₂O (10 mL). The aqueous layer was extracted with EtOAc (2×20 mL) and the combined organics were washed with brine, dried (MgSO₄), filtered and concentrated in vacuo. Purification by column chromatography (MeOH-DCM-NH₄OH, 2:97:1), followed by separation of the enantiomers by chiral HPLC afforded the title compound: RT=2.80 min, m/z (ES⁺)=474.19 [M+H]⁺.

### Example 10

### (S)-1-[(S)-2- Amino-3-(2-fluoro-4-{3-[1-(3-isopropyl-[1,2,4]oxadiazol-5-yl)-piperidin-4-yl]propoxy}phenyl)propionyl]pyrrolidine-2-carbonitrile hydrochloride

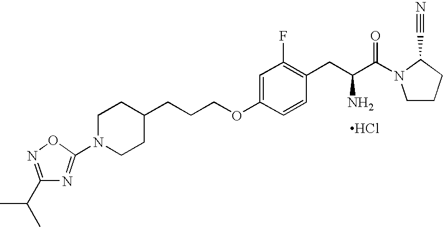

TFA (8 mL) was added to a solution of [(S)-2-((S)-2-cyanopyrrolidin-1-yl)-1-(2-fluoro-4-{3-[1-(3-isopropyl-[1,2,4]oxadiazol-5-yl)piperidin-4-yl]propoxy}benzyl)-2-oxoethyl]-carbamic acid tert-butyl ester (Preparation 51, 500 mg, 815 μmol) in DCM (40 mL) at 0° C. and stirred at ambient temperature for 3 h. Saturated aqueous Na₂CO₃ solution was added until the aqueous layer remained at pH 10, then dilute NaOH was added to adjust the pH to pH 12. The organic layer was washed with brine, dried (MgSO₄), filtered and concentrated in vacuo. Purification by column chromatography (MeOH-DCM, 1:19) afforded (S)-1-[(S)-2-amino-3-(2-fluoro-4-{3-[1-(3-isopropyl-[1,2,4]oxadiazol-5-yl)piperidin-4-yl]propoxy}phenyl)propionyl]-pyrrolidine-2-carbonitrile. This compound was dissolved in Et₂O/dioxane (5:1) and excess 4M HCl in dioxane was added. The solvent was removed in vacuo and the remainder was triturated with Et₂O to afford the title compound: RT=2.97 min; m/z (ES⁺)=513.22 [M+H]⁺.

### Example 11

### 2-Amino-2-(4-{3-[1-(3-isopropyl-[1,2,4]oxadiazol-5-yl)piperidin-4-yl]propoxy}-phenyl)-1-pyrrolidin-1-ylethanone

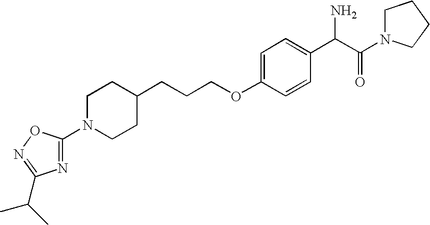

The title compound was synthesized from [1-(4-{3-[1-(3-isopropyl-[1,2,4]oxadiazol-5-yl)piperidin-4-yl]propoxy}phenyl)-2-oxo-2-pyrrolidin-1-ylethyl]carbamic acid tert-butyl ester (Preparation 52, 80.0 mg, 140 μmol) employing a procedure similar to that outlined in Example 6: RT=2.90 min; m/z (ES⁺)=456.36 [M+H]⁺

### Example 12

### (S)-2-Amino-3-(2-fluoro-4-{(R)-3-[1-(3-isopropyl-[1,2,4]oxadiazol-5-yl)-piperidin-4-yl]butoxy}phenyl)-1-thiazolidin-3-ylpropan-1-one hydrochloride

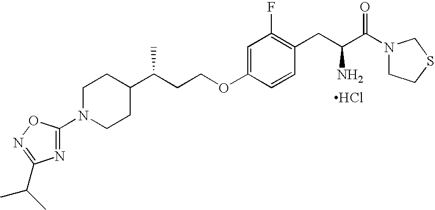

[(S)-1-(2-Fluoro-4-{(R)-3-[1-(3-isopropyl-[1,2,4]oxadiazol-5-yl)piperidin-4-yl]-butoxy}benzyl)-2-oxo-2-thiazolidin-3-ylethyl]carbamic acid tert-butyl ester was isolated from [1-(2-fluoro-4-{(R)-3-[1-(3-isopropyl-[1,2,4]oxadiazol-5-yl)piperidin-4-yl]butoxy}benzyl)-2-oxo-2-thiazolidin-3-ylethyl]carbamic acid tert-butyl ester (Preparation 55) by chiral-HPLC. To a solution of this compound (188 mg, 304 μmol) in DCM (9 mL) was added TFA (1 mL) and the resulting solution was stirred at ambient temperature for 1 h. The reaction mixture was concentrated in vacuo and the residue was dissolved in DCM (4 mL) and MeOH (4 mL), before adding MP-carbonate Resin (200 mg, 600 μmol). The reaction mixture was stirred at ambient temperature for 1 h, prior to filtration and concentration of the filtrate in vacuo to afford (S)-2-amino-3-(2-fluoro-4-{(R)-3-[1-(3-isopropyl-[1,2,4]oxadiazol-5-yl)piperidin-4-yl]butoxy}-phenyl)-1-thiazolidin-3-ylpropan-1-one. This compound was dissolved in Et₂O (5 mL) and 4M HCl in dioxane (200 μL) was added. The solvent was removed in vacuo and the remainder was washed with Et₂O (2×5 mL) to afford the title compound: RT=3.02 min; m/z (ES⁺)=520.2 [M+H]⁺.

### Example 13

### (S)-1-[(S)-2-Amino-3-(2-fluoro-4-{(R)-3-[1-(3-isopropyl-[1,2,4]oxadiazol-5-yl)-piperidin-4-yl]butoxy}phenyl)propionyl]pyrrolidine-2-carbonitrile hydrochloride

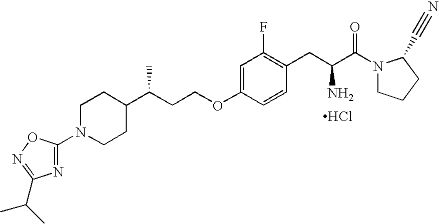

TFA (2.5 mL) was added to a solution of [(S)-2-((S)-2-cyanopyrrolidin-1-yl)-1-(2-fluoro-4-{(R)-3-[1-(3-isopropyl-[1,2,4]oxadiazol-5-yl)piperidin-4-yl]butoxy}benzyl)-2-oxo-ethyl]carbamic acid tert-butyl ester (Preparation 62, 500 mg, 799 μmol) in DCM (22.5 mL) and the resulting solution was stirred at ambient temperature for 1 h before adding additional DCM (200 mL) and washing with saturated aqueous NaHCO₃ solution (2×150 mL) and brine (200 mL). The organics were dried (MgSO₄), filtered, concentrated in vacuo and purified by column chromatography (MeOH-DCM, 1:49) to afford (S)-1-[(S)-2-amino-3-(2-fluoro-4-{(R)-3-[1-(3-isopropyl-[1,2,4]oxadiazol-5-yl)piperidin-4-yl]butoxy}phenyl)propionyl]pyrrolidine-2-carbonitrile. This compound was dissolved in Et₂O (20 mL) and 4M HCl in dioxane (1 mL) was added. The solvent was removed in vacuo to afford the title compound: RT=2.97 min; m/z (ES⁺)=527.2 [M+H]⁺.

### Example 14

### (S)-2-Amino-1-(3,3-difluoroazetidin-1-yl)-3-(2-fluoro-4-{(R)-3-[1-(3-isopropyl-[1,2,4]oxadiazol-5-yl)piperidin-4-yl]butoxy}phenyl)propan-1-one tosylate

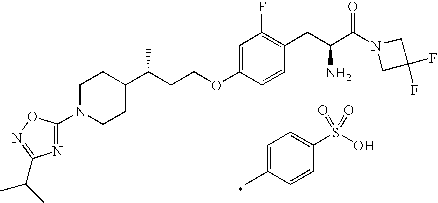

TFA (1 mL) was added to a solution of [(S)-2-(3,3-difluoroazetidin-1-yl)-1-(2-fluoro-4-{(R)-3-[1-(3-isopropyl-[1,2,4]oxadiazol-5-yl)piperidin-4-yl]butoxy}benzyl)-2-oxoethyl]-carbamic acid tert-butyl ester (Preparation 63, 258 mg, 414 μmol) in DCM (19 mL) and the resulting solution was stirred at ambient temperature for 1.5 h before adding additional DCM (100 mL) and washing with saturated aqueous NaHCO₃ solution (75 mL) and brine (50 mL). The organics were dried (MgSO₄), filtered, concentrated in vacuo and purified by column chromatography (MeOH-DCM, 1:24) to afford (S)-2-amino-1-(3,3-difluoroazetidin-1-yl)-3-(2-fluoro-4-{(R)-3-[1-(3-isopropyl-[1,2,4]oxadiazol-5-yl)piperidin-4-yl]butoxy}phenyl)propan-1-one. This compound was dissolved in (DCM-MeOH, 1:1) (10 mL) and toluene-4-sulfonic acid (58.0 mg, 337 μmol) was added. The solvent was removed in vacuo and the remainder was triturated with Et₂O to afford the title compound: RT=3.00 min; m/z (ES⁺)=524.2 [M+H]⁺.

### Example 15

### (S)-2-Amino-1-(3,3-difluoropyrrolidin-1-yl)-3-(2-fluoro-4-{(R)-3-[1-(3-isopropyl-[1,2,4]oxadiazol-5-yl)piperidin-4-yl]butoxy}phenyl)propan-1-one tosylate

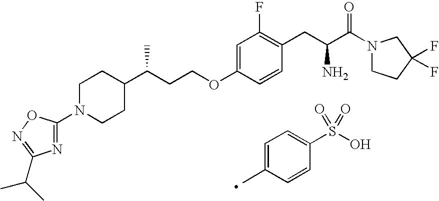

The title compound was synthesized from [(S)-2-(3,3-difluoropyrrolidin-1-yl)-1-(2-fluoro-4-{(R)-3-[1-(3-isopropyl-[1,2,4]oxadiazol-5-yl)piperidin-4-yl]butoxy}benzyl)-2-ethyl]carbamic acid tert-butyl ester (Preparation 64, 252 mg, 414 μmol) employing a procedure similar to that outlined in Example 14: RT=3.05 min; m/z (ES⁺)=538.2 [M+H]⁺.

### Example 16

### (3,3-Difluoropyrrolidin-1-yl)-((S)-7-{(R)-3-[1-(3-isopropyl-[1,2,4]oxadiazol-5-yl)piperidin-4-yl]butoxy}-1,2,3,4-tetrahydroisoquinolin-3-yl)methanone hydrochloride

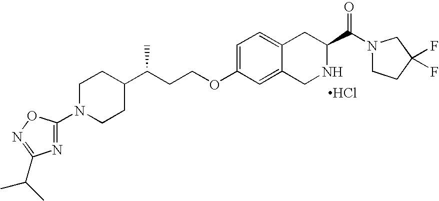

The title compound was synthesized from (S)-3-(3,3-difluoropyrrolidine-1-carbonyl)-7-{(R)-3-[1-(3-isopropyl-[1,2,4]oxadiazol-5-yl)piperidin-4-yl]butoxyl-3,4-dihydro-1H-isoquinoline-2-carboxylic acid tert-butyl ester (Preparation 68, 70.0 mg, 111 μmol) employing a procedure similar to that outlined in Example 13: RT=3.13 min; m/z (ES⁺)=532.4 [M+H]⁺.

### Example 17

### (S)-1-((S)-7-{(R)-3-[1-(3-Isopropyl-[1,2,4]oxadiazol-5-yl)piperidin-4-yl]butoxy}-1,2,3,4-tetrahydroisoquinoline-3-carbonyl)pyrrolidine-2-carbonitrile hydrochloride

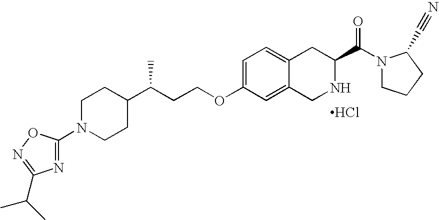

The title compound was synthesized from (S)-3-((S)-2-cyanopyrrolidine-1-carbonyl)-7-{(R)-3-[1-(3-isopropyl-[1,2,4]oxadiazol-5-yl)piperidin-4-yl]butoxy}-3,4-dihydro-1H-isoquinoline-2-carboxylic acid tert-butyl ester (Preparation 70, 22.0 mg, 35.0 μmol) employing a procedure similar to that outlined in Example 13: RT=2.90 min; m/z (ES⁺)=521.3 [M+H]⁺.

### Example 18

### (S)-2-Amino-1-(3,3-difluoropyrrolidin-1-yl)-3-(2-fluoro-4-{3-[1-(3-isopropyl-[1,2,4]oxadiazol-5-yl)piperidin-4-yl]propoxy}phenyl)propan-1-one tosylate

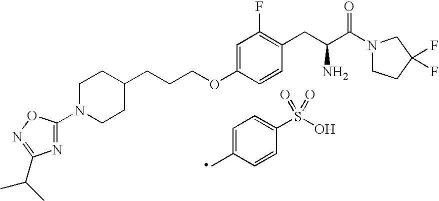

The title compound was synthesized from [(S)-2-(3,3-difluoropyrrolidin-1-yl)-1-(2-fluoro-4-{3-[1-(3-isopropyl-[1,2,4]oxadiazol-5-yl)piperidin-4-yl]propoxy}benzyl)-2-oxoethyl]-carbamic acid tert-butyl ester (Preparation 72, 136 mg, 218 μmol) employing a procedure similar to that outlined in Example 14: RT=4.43 min; m/z (ES⁺)=524.18 [M+H]⁺.

### Example 19

### (S)-2-Amino-3-(2-fluoro-4-{3-[1-(3-isopropyl-[1,2,4]oxadiazol-5-yl)piperidin-4-yl]propoxy}phenyl)-1-((S)-3-fluoropyrrolidin-1-yl)propan-1-one tosylate

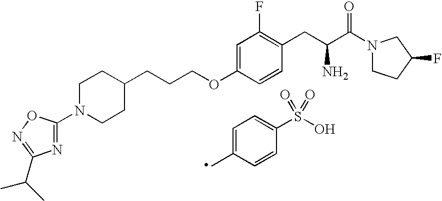

The title compound was synthesized from [(S)-1-(2-fluoro-4-{3-[1-(3-isopropyl-[1,2,4]oxadiazol-5-yl)piperidin-4-yl]propoxy}benzyl)-2-((S)-3-fluoropyrrolidin-1-yl)-2-oxo-ethyl]carbamic acid tert-butyl ester (Preparation 73, 140 mg, 231 μmol) employing a procedure similar to that outlined in Example 14: RT=2.93 min; m/z (ES⁺)=506.17 [M+H]⁺.

Example 20

### (2S,3S)-2-Amino-1-(3,3-difluoropyrrolidin-1-yl)-3-(2-fluoro-4-{(R)-3-[1-(3-isopropyl-[1,2,4]oxadiazol-5-yl)piperidin-4-yl]butoxy}phenyl)butan-1-one tosylate

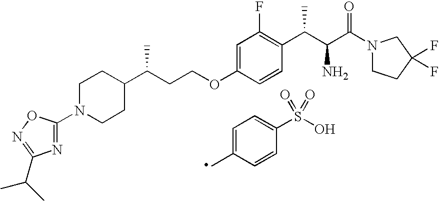

The title compound was synthesized from [(1S,2S)-1-(3,3-difluoropyrrolidine-1-carbonyl)-2-(2-fluoro-4-{(R)-3-[1-(3-isopropyl-[1,2,4]oxadiazol-5-yl)piperidin-4-yl]butoxy}-phenyl)propyl]carbamic acid tert-butyl ester (Preparation 85) employing a procedure similar to that outlined in Example 14: RT=3.04 min; m/z (ES⁺)=552.22 [M+H]⁺.

### Example 21

### (S)-1-[(2S,3S)-2-Amino-3-(2-fluoro-4-{(R)-3-[1-(3-isopropyl-[1,2,4]oxadiazol-5-yl)piperidin-4-yl]butoxy}phenyl)butyryl]pyrrolidine-2-carbonitrile

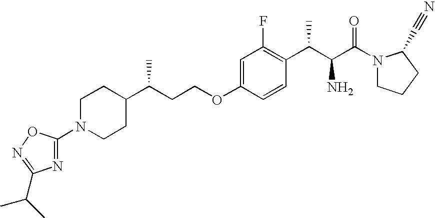

The title compound was synthesized from [(1S,2S)-1-((S)-2-cyanopyrrolidine-1-carbonyl)-2-(2-fluoro-4-{(R)-3-[1-(3-isopropyl-[1,2,4]oxadiazol-5-yl)piperidin-4-yl]butoxy}-phenyl)propyl]carbamic acid tert-butyl ester (Preparation 87, 184 mg, 287 μmol) employing a procedure similar to that outlined in Example 6: RT=3.17 min; m/z (ES⁺)=541.37 [M+H]⁺.

### Example 22

### 4-(3-{4-[(1S,2S)-2-Amino-3-(3,3-difluoropyrrolidin-1-yl)-1-methyl-3-oxo-propyl]-3-fluorophenoxy}propyl)piperidine-1-carboxylic acid isopropyl ester hydrochloride

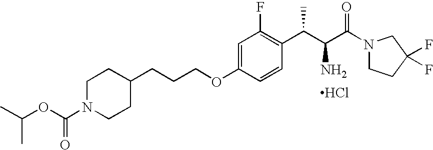

The title compound was synthesized from 4-(3-{4-[(1S,2S)-2-tert-butoxycarbonylamino-3-(3,3-difluoropyrrolidin-1-yl)-1-methyl-3-oxopropyl]-3-fluoro-phenoxy}propyl)piperidine-1-carboxylic acid isopropyl ester (Preparation 90, 67.0 mg, 110 μmol) employing a procedure similar to that outlined in Example 13: RT=3.06 min; m/z (ES⁺)=514.24 [M+H]⁺.

### Example 23

### (S)-1-[(S)-2-Amino-2-(4-{(R)-3-[1-(3-isopropyl-[1,2,4]oxadiazol-5-yl)piperidin-4-yl]butoxy}phenyl)acetyl]pyrrolidine-2-carbonitrile tosylate

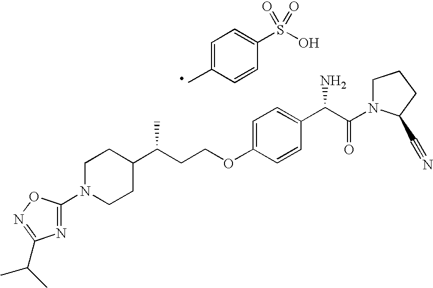

The title compound was synthesized from [(S)-2-((S)-2-cyanopyrrolidin-1-yl)-1-(4-{(R)-3-[1-(3-isopropyl-[1,2,4]oxadiazol-5-yl)piperidin-4-yl]butoxy}phenyl)-2-oxoethyl]carbamic acid tert-butyl ester (Preparation 94, 322 mg, 541 μmol) employing a procedure similar to that outlined in Example 14: RT=2.97 min; m/z (ES⁺)=495.27 [M+H]⁺.

### Example 24

### (S)-2-Amino-1-(3,3-difluoropyrrolidin-1-yl)-2-(4-{(R)-3-[1-(3-isopropyl-[1,2,4]oxadiazol-5-yl)piperidin-4-yl]butoxy}phenyl)ethanone tosylate

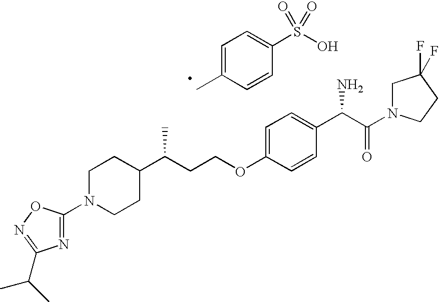

The title compound was synthesized from [(S)-2-(3,3-difluoropyrrolidin-1-yl)-1-(4-{(R)-3-[1-(3-isopropyl-[1,2,4]oxadiazol-5-yl)piperidin-4-yl]butoxy}phenyl)-2-oxoethyl]-carbamic acid tert-butyl ester (Preparation 95, 88.0 mg, 145 μmol) employing a procedure similar to that outlined in Example 14: RT=3.13 min; m/z (ES⁺)=506.35 [M+H]⁺.

### Example 25

### (S)-1-[(S)-2-Amino-3-(4-{3-[1-(3-isopropyl-[1,2,4]oxadiazol-5-yl)piperidin-4-yl]-propoxy}-2-methylphenyl)propionyl]pyrrolidine-2-carbonitrile tosylate

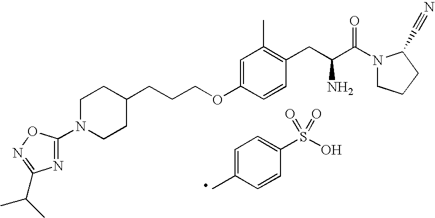

The title compound was synthesized from [(S)-2-((S)-2-cyanopyrrolidin-1-yl)-1-(4-{3-[1-(3-isopropyl-[1,2,4]oxadiazol-5-yl)piperidin-4-yl]propoxy}-2-methylbenzyl)-2-oxoethyl]-carbamic acid tert-butyl ester (Preparation 99, 90.0 mg, 148 μmol) employing a procedure similar to that outlined in Example 14: RT=2.93 min; m/z (ES⁺)=509.31 [M+H]⁺.

### Example 26

### (S)-2-Amino-1-(3,3-difluoropyrrolidin-1-yl)-2-(2-fluoro-4-{(R)-3-[1-(3-isopropyl-[1,2,4]oxadiazol-5-yl)piperidin-4-yl]butoxy}phenyl)ethanone tosylate

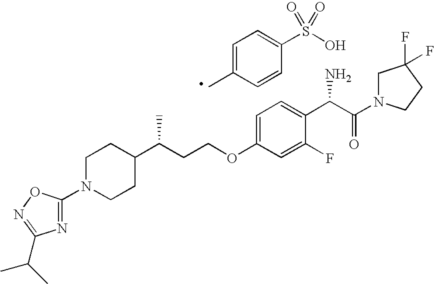

The title compound was synthesized from [(S)-2-(3,3-difluoropyrrolidin-1-yl)-1-(2-fluoro-4-{(R)-3-[1-(3-isopropyl-[1,2,4]oxadiazol-5-yl)piperidin-4-yl]butoxy}phenyl)-2-oxo-ethyl]carbamic acid tert-butyl ester (Preparation 107, 36.0 mg, 60 μmol) employing a procedure similar to that outlined in Example 14: RT=3.00 min; m/z (ES⁺)=524.3 [M+H]⁺.

### Example 27

### (S)-1-[(S)-2-Amino-2-(2-fluoro-4-{(R)-3-[1-(3-isopropyl-[1,2,4]oxadiazol-5-yl)-piperidin-4-yl]butoxy}phenyl)acetyl]pyrrolidine-2-carbonitrile tosylate

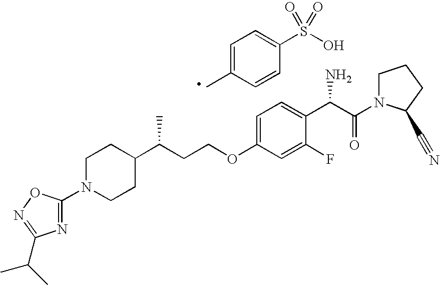

The title compound was synthesized from [(S)-2-((S)-2-cyanopyrrolidin-1-yl)-1-(2-fluoro-4-{(R)-3-[1-(3-isopropyl-[1,2,4]oxadiazol-5-yl)piperidin-4-yl]butoxy}phenyl)-2-oxo-ethyl]carbamic acid tert-butyl ester (Preparation 109, 115 mg, 190 μmol) employing a procedure similar to that outlined in Example 14: RT=3.06 min; m/z (ES⁺)=513.3 [M+H]⁺.

### Example 28

### (S)-1-[(S)-2-Amino-2-(4-{(R)-3-[1-(3-isopropyl-[1,2,4]oxadiazol-5-yl)piperidin-4-yl]butoxy}-2-methylphenyl)acetyl]pyrrolidine-2-carbonitrile trifluoroacetate

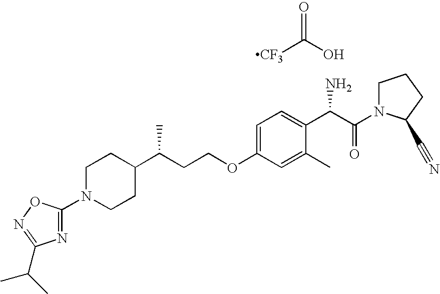

TFA (1 mL) was added to a solution of [(S)-2-((S)-2-cyanopyrrolidin-1-yl)-1-(4-{(R)-3-[1-(3-isopropyl-[1,2,4]oxadiazol-5-yl)piperidin-4-yl]butoxy}-2-methylphenyl)-2-oxoethyl]-carbamic acid tert-butyl ester (Preparation 118, 125 mg, 210 μmol) in DCM (8 mL) at 0° C. and the resulting solution was stirred at this temperature for 2.5 h, before the addition of further TFA (1 mL). Stirring was continued at 0° C. for 45 min before adding additional DCM (40 mL) and washing with saturated aqueous NaHCO₃ solution (50 mL) and brine (30 mL). The organics were dried (MgSO₄), filtered and concentrated in vacuo. The crude product was purified by HPLC to afford the title compound: RT=3.11 min; m/z (ES⁺)=509.4 [M+H]⁺.

### Example 29

### (S)-2-Amino-3-(4-{(R)-4-[1-(5-chloropyrimidin-2-yl)piperidin-4-yl]pentyl}-2-fluorophenyl)-1-(3,3-difluoropyrrolidin-1-yl)propan-1-one hydrochloride

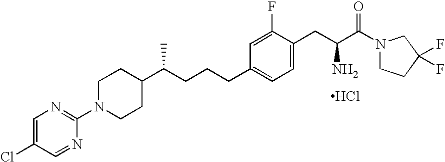

4M HCl in dioxane (1 mL) was added to [(S)-1-(4-{(R)-4-[1-(5-chloropyrimidin-2-yl)-piperidin-4-yl]pentyl}-2-fluorobenzyl)-2-(3,3-difluoropyrrolidin-1-yl)-2-oxoethyl]carbamic acid tert-butyl ester (Preparation 126, 4.00 mg, 6.20 μmol) and the resulting reaction mixture was stirred at ambient temperature for 0.5 h. The solvent was removed in vacuo to afford the title compound: RT=3.58 min; m/z (ES⁺)=538.34 [M+H]⁺.

### Example 30

### (R)-2-(2,5-Difluorophenyl)-1-(4-{(R)-3-[1-(3-isopropyl-[1,2,4]oxadiazol-5-yl)-piperidin-4-yl]butoxy}phenyl)ethylamine

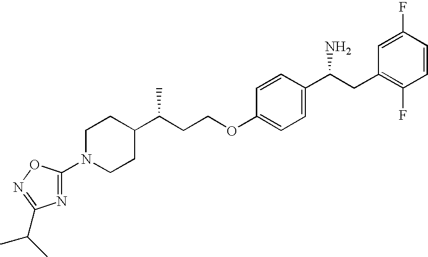

LiHMDS (1M in THF, 1.62 mL, 1.62 mmol) was added to a solution of 4-{(R)-3-[1-(3-isopropyl-[1,2,4]oxadiazol-5-yl)piperidin-4-yl]butoxy}benzaldehyde (Preparation 127, 500 mg, 1.35 mmol) in THF (0.5 mL) at 0° C. and the resulting solution was stirred at this temperature for 0.5 h. 2,5-Difluorobenzylmagnesium bromide (6.46 mL, 1.62 mmol) in THF (0.5 mL) was added and the resulting reaction mixture was stirred at ambient temperature for 3 h before quenching with sauturated aqueous ammonium chloride solution. The reaction mixture was extracted with DCM (3×) and the combined organic extracts were dried (MgSO₄), filtered and concentrated in vacuo. Purification by column chromatography (EtOAc-IH, 2:3 to 4:1) then (DCM-MeOH-NH₃, 95:5:1) afforded 2-(2,5-difluorophenyl)-1-(4-{(R)-3-[1-(3-isopropyl-[1,2,4]oxadiazol-5-yl)piperidin-4-yl]butoxy}phenyl)ethylamine. Purification by chiral-HPLC afforded the title compound: RT=2.18 min; m/z (ES⁺)=499.31 [M+H]⁺.

### Example 31

### (S)-2-Amino-2-(4-{(R)-3-[1-(5-chloropyrimidin-2-yl)piperidin-4-yl]butoxy}-phenyl)-1-(3,3-difluoropyrrolidin-1-yl)ethanone

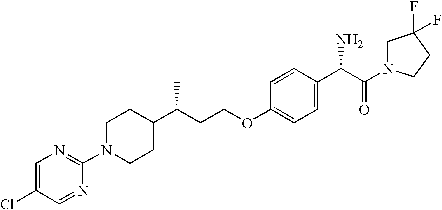

The title compound was synthesised from [(S)-1-(4-{(R)-3-[1-(5-chloropyrimidin-2-yl)-piperidin-4-yl]butoxy}phenyl)-2-(3,3-difluoropyrrolidin-1-yl)-2-oxoethyl]carbamic acid tert-butyl ester (Preparation 130, 77.0 mg, 127 μmol) employing a procedure similar to that outlined in Example 3: RT=3.23 min; m/z (ES⁺)=508.11 [M+H]⁺.

### Example 32

### (S)-1-[(S)-2-Amino-2-(4-{(R)-3-[1-(3-ethyl-[1,2,4]oxadiazol-5-yl)piperidin-4-yl]-butoxy}phenyl)acetyl]pyrrolidine-2-carbonitrile

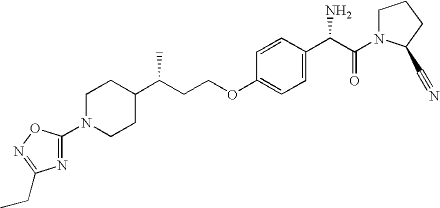

The title compound was synthesised from [(S)-2-((S)-2-cyanopyrrolidin-1-yl)-1-(4-{(R)-3-[1-(3-ethyl-[1,2,4]oxadiazol-5-yl)piperidin-4-yl]butoxy}phenyl)-2-oxoethyl]carbamic acid tent-butyl ester (Preparation 135, 467 mg, 804 μmol) employing a procedure similar to that outlined in Example 3: RT=3.04 min; m/z (ES⁺)=481.25 [M+H]⁺.

### Example 33

### (S)-1-[(S)-2-Amino-2-(2-fluoro-4-{3-[1-(3-isopropyl-[1,2,4]oxadiazol-5-yl)-piperidin-4-yl]propoxy}phenyl)acetyl]pyrrolidine-2-carbonitrile

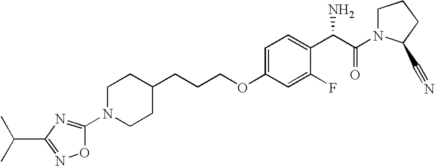

A solution of ceric ammonium nitrate (211 mg, 384 μmol) in H₂O (2 mL) was added to a solution of (S)-1-[2-(2-fluoro-4-{3-[1-(3-isopropyl-[1,2,4]oxadiazol-5-yl)piperidin-4-yl]-propoxy}phenyl)-2-(4-methoxyphenylamino)acetyl]pyrrolidine-2-carbonitrile (Preparation 138, 116 mg, 192 μmol) in MeCN (10 mL), and the resulting solution was stirred at ambient temperature for 30 min. Saturated aqueous Na₂S₂O₃ solution was added and the mixture was extracted with EtOAc (3×30 mL). The combined organic extracts were extracted with 1M HCl (2×20 mL), then the combined acidic extracts were neutralized with 2M NaOH and extracted with EtOAc (5×20 mL). The combined organic extracts were washed with brine (20 mL), dried (MgSO₄), filtered and concentrated in vacuo. Purification by column chromatography (MeOH-DCM-NH₃, 10:389:1) afforded the title compound: RT=2.83 min, m/z (ES⁺)=499.26 [M+H]⁺.

The following compounds can be prepared by analogous processes to those described above:

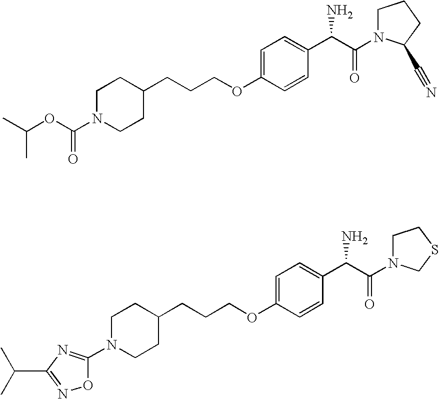

The biological activity of the compounds of the invention may be tested in the following assay systems:

### GPR119 Yeast Reporter Assay

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Ste3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μL of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di((β-D-galactopyranoside) was used (FDG), a substrate for the enzyme that releases fluorescein, allowing a fluorimetric read-out. 20 μL per well of 500 μM FDG/2.5% Triton X100 was added (the detergent was necessary to render the cells permeable). After incubation of the cells with the substrate for 60 min, 20 μL per well of 1M sodium carbonate was added to terminate the reaction and enhance the fluorescent signal. The plates were then read in a fluorimeter at 485/535 nm.

The compounds of the invention give an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line was used to investigate the effect of compounds of the invention on intracellular levels of cyclic AMP (cAMP). The cell monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol.

Compounds of the invention produced a concentration-dependent increase in intracellular cAMP level and generally had an EC₅₀ of <10 μM. Compounds showing and EC₅₀ of less than 1 μM in the cAMP assay may be preferred.

### DPP-IV Assay Method

DPP-IV activity was measured by monitoring the cleavage of the fluorogenic peptide substrate, H-Gly-Pro-7-amino-4-methylcoumarin (GP-AMC) whereby the product 7-amino-4-methylcoumarin is quantified by fluorescence at excitation 380 nm and emission 460 nm. Assays were carried out in 96-well plates (Black OptiPlate-96F) in a total volume of 100 μL per well consisting of 50 mM Tris pH 7.6, 100 μM GP-AMC, 10-25 μU recombinant human DPP-IV and a range of inhibitor dilutions in a final concentration of 1% DMSO. Plates were read in a fluorimeter after 30 min incubation at 37° C. Recombinant human DPP-IV residues Asn29-Pro766 was purchased from BioMol.

Compounds of the invention generally had a Ki of <10 μM.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 mL/well and cultured for 24 h and the medium was then discarded. Cells were incubated for 15 min at room temperature with 100 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 uL lysis buffer (5 mM HEPES,0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μL reactions were set up containing 8 μL sample, 5 μL acceptor bead mix and 12 μL detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC₅₀ of less than 10 μM. Compounds showing an EC₅₀ of less than 1 μM in the cAMP assay may be preferred.

### Insulin Secretion Assay

HIT-T15 cells are plated in standard culture medium in 12-well plates at 106 cells/1 ml/well and cultured for 3 days and the medium then discarded. Cells are washed ×2 with supplemented Krebs-Ringer buffer (KRB) containing 119 mM NaCl, 4.74 mM KCl, 2.54 mM CaCl₂, 1.19 mM MgSO₄, 1.19 mM KH₂PO₄, 25 mM NaHCO₃, 10 mM HEPES at pH 7.4 and 0.1% bovine serum albumin. Cells are incubated with 1 ml KRB at 37° C. for 30 min which is then discarded. This is followed by a second incubation with KRB for 30 min, which is collected and used to measure basal insulin secretion levels for each well. Compound dilutions (0, 0.1, 0.3, 1, 3, 10 μM) are then added to duplicate wells in 1 ml KRB, supplemented with 5.6 mM glucose. After 30 min incubation at 37° C. samples are removed for determination of insulin levels. Measurement of insulin was done using the Mercodia Rat insulin ELISA kit, following the manufacturers' instructions, with a standard curve of known insulin concentrations. For each well, insulin levels are corrected by subtraction of the basal secretion level from the pre-incubation in the absence of glucose. Data is analysed using XLfit 3 software.

Compounds of the invention preferably increase insulin secretion at an EC₅₀ of less than 10 μM.

### Oral Glucose Tolerance Tests

The effects of compounds of the invention on oral glucose (Glc) tolerance were evaluated in male Sprague-Dawley rats. Food was withdrawn 16 h before administration of Glc and remained withdrawn throughout the study. Rats had free access to water during the study. A cut was made to the animals' tails, then blood (1 drop) was removed for measurement of basal Glc levels 60 min before administration of the Glc load. Then, the rats were weighed and dosed orally with test compound or vehicle (20% aqueous hydroxypropyl-β-cyclodextrin) 45 min before the removal of an additional blood sample and treatment with the Glc load (2 g kg⁻¹ p.o.). Blood samples were then taken from the cut tip of the tail 5, 15, 30, 60, 120, and 180 min after Glc administration. Blood glucose levels were measured just after collection using a commercially available glucose-meter (OneTouch® Ultra™ from Lifescan). Representative compounds of the invention statistically reduced the Glc excursion at doses of <100 mg kg⁻¹.

The effects of compounds of the invention on oral glucose (Glc) tolerance may also be evaluated in male C57B1/6 or male oblob mice. Food is withdrawn 5 h before administration of Glc and remained withdrawn throughout the study. Mice have free access to water during the study. A cut was made to the animals' tails, then blood (20 μL) is removed for measurement of basal Glc levels 45 min before administration of the Glc load. Then, the mice are weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 uL) are then taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood are then added to 180 μL of Trinders glucose reagent (Sigma enzymatic (Trinder) colorimetric method) in a 96-well assay plate. After mixing, the samples are left at room temperature for 30 min before being read against Glc standards (Sigma glucose/urea nitrogen combined standard set). Compounds of the invention preferably statistically reduce the Glc excursion at doses ≦100 mg kg⁻¹.

What is claimed is:

1. A compound which is an agonist of GPR119 and an inhibitor of DPP-IV, or a pharmaceutically acceptable salt thereof.

2. A compound according to claim 1 which comprises an α-aminoacylpyrrolidine or α-aminoacylthiazolidine group.

3. A compound of formula (I), or a pharmaceutically acceptable salt thereof: 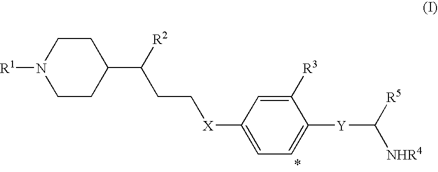
wherein R¹ is —C(O)—O—C₂₋₄ alkyl, or R¹ is: 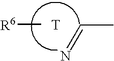
where T together with the —N═C— to which it is attached forms a 5- or 6-membered heteroaryl ring optionally containing up to 2 additional heteroatoms selected from N, O and S;
when T together with the —N═C— to which it is attached forms a 5-membered heteroaryl ring, R⁶ is C₂₋₄ alkyl, and when T together with the —N═C— to which it is attached forms a 6-membered heteroaryl ring, R⁶ is C₂₋₄ alkyl, fluoro or chloro;
R² is hydrogen or methyl;
R³ is hydrogen, fluoro or chloro, or when R⁷ is cyano, R³ may be methyl;
R⁴ is hydrogen or, when Y is —CH₂— or —CHMe-, R⁴ can be —CH₂— linked to position * on the phenyl ring to form a fused 6-membered N-containing heterocycle;
R⁵ is benzyl optionally substituted by one or more fluoro, chloro, cyano or methyl groups, or R⁵ is: 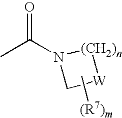
where n is 1 or 2 and m is 0, 1 or 2;
W is CH₂ or, when n is 2, W may be S;
when W is CH₂, R⁷ is fluoro or cyano, and when W is S, R⁷ is cyano;
X is —O— or —CH₂—; and
Y is a bond, —CH₂— or —CHMe-.

4. A compound according to claim 3, or a pharmaceutically acceptable salt thereof, wherein R¹ is —C(O)—O-isopropyl or —C(O)—O-tert-butyl, or R¹ is: 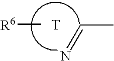
where T together with the —N═C— to which it is attached forms a 5- or 6-membered heteroaryl ring optionally containing up to 2 additional heteroatoms selected from N and O.

5. A compound according to claim 3, or a pharmaceutically acceptable salt thereof, wherein when R² is methyl the stereochemistry at the carbon to which it is attached is in the (R)-configuration.

6. A compound according to claim 3, or a pharmaceutically acceptable salt thereof, wherein R³ is hydrogen or fluoro.

7. A compound according to claim 3, or a pharmaceutically acceptable salt thereof, wherein R⁵ is:

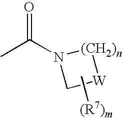

8. A compound according to claim 7, or a pharmaceutically acceptable salt thereof, wherein n is 2.

9. A compound according to claim 7, or a pharmaceutically acceptable salt thereof, wherein W is CH₂.

10. A compound according to of claim 3, or a pharmaceutically acceptable salt thereof, wherein Y is —CH₂— or —CHMe-.

11. A compound according to claim 3, or a pharmaceutically acceptable salt thereof, wherein the stereochemistry of the compound is as shown below:

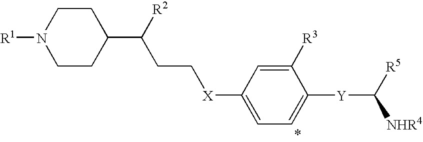

12. A compound according to claim 3, selected from:
(S)-2-Amino-3-(4-{3-[1-(3-isopropyl-[1,2,4]oxadiazol-5-yl)piperidin-4-yl]-propoxy}phenyl)-1-thiazolidin-3-ylpropan-1-one;
(S)-2-Amino-1-((S)-3-fluoropyrrolidin-1-yl)-3-(4-{3-[1-(3-isopropyl-[1,2,4]oxadiazol-5-yl)piperidin-4-yl]propoxy}phenyl)propan-1-one;
(S)-1-[(S)-2-Amino-2-(4-{3-[1-(3-isopropyl-[1,2,4]oxadiazol-5-yl)piperidin-4-yl]-propoxy}phenyl)acetyl]pyrrolidine-2-carbonitrile;
(S)-1-[(R)-2-Amino-2-(4-{3-[1-(3-isopropyl-[1,2,4]oxadiazol-5-yl)piperidin-4-yl]-propoxy}phenyl)acetyl]pyrrolidine-2-carbonitrile;
(S)-2-Amino-3-(4-{(R)-3-[1-(3-isopropyl-[1,2,4]oxadiazol-5-yl)piperidin-4-yl]-butoxy}phenyl)-1-thiazolidin-3-ylpropan-1-one;
(S)-1-[(S)-2-Amino-3-(4-{(R)-3-[1-(3-isopropyl-[1,2,4]oxadiazol-5-yl)piperidin-4-yl]butoxy}phenyl)propionyl]pyrrolidine-2-carbonitrile;
(S)-1-[(S)-2-Amino-3-(4-{3-[1-(3-isopropyl-[1,2,4]oxadiazol-5-yl)piperidin-4-yl]-propoxy}phenyl)propionyl]pyrrolidine-2-carbonitrile;
(S)-2-Amino-3-(4-{3-[1-(3-isopropyl-[1,2,4]oxadiazol-5-yl)piperidin-4-yl]-propoxy}phenyl)-1-pyrrolidin-1-ylpropan-1-one;
(S)-2-Amino-2-(2-fluoro-4-{3-[1-(3-isopropyl-[1,2,4]oxadiazol-5-yl)piperidin-4-yl]propoxy}phenyl)-1-pyrrolidin-1-ylethanone;
(S)-1-[(S)-2-Amino-3-(2-fluoro-4-{3-[1-(3-isopropyl-[1,2,4]oxadiazol-5-yl)piperidin-4-yl]propoxy}phenyl)propionyl]pyrrolidine-2-carbonitrile;
2-Amino-2-(4-{3-[1-(3-isopropyl-[1,2,4]oxadiazol-5-yl)piperidin-4-yl]propoxy}-phenyl)-1-pyrrolidin-1-ylethanone;
(S)-2-Amino-3-(2-fluoro-4-{(R)-3-[1-(3-isopropyl-[1,2,4]oxadiazol-5-yl)piperidin-4-yl]butoxy}phenyl)-1-thiazolidin-3-ylpropan-1-one;
(S)-1-[(S)-2-Amino-3-(2-fluoro-4-{(R)-3-[1-(3-isopropyl-[1,2,4]oxadiazol-5-yl)-piperidin-4-yl]butoxy}phenyl)propionyl]pyrrolidine-2-carbonitrile;
(S)-2-Amino-1-(3,3-difluoroazetidin-1-yl)-3-(2-fluoro-4-{(R)-3-[1-(3-isopropyl-[1,2,4]oxadiazol-5-yl)piperidin-4-yl]butoxy}phenyl)propan-1-one;
(S)-2-Amino-1-(3,3-difluoropyrrolidin-1-yl)-3-(2-fluoro-4-{(R)-3-[1-(3-isopropyl-[1,2,4]oxadiazol-5-yl)piperidin-4-yl]butoxy}phenyl)propan-1-one;
(3,3-Difluoropyrrolidin-1-yl)-((S)-7-{(R)-3-[1-(3-isopropyl-[1,2,4]oxadiazol-5-yl)piperidin-4-yl]butoxy}-1,2,3,4-tetrahydroisoquinolin-3-yl)methanone;
(S)-1-((S)-7-{(R)-3-[1-(3-Isopropyl-[1,2,4]oxadiazol-5-yl)piperidin-4-yl]butoxy}-1,2,3,4-tetrahydroisoquinoline-3-carbonyl)pyrrolidine-2-carbonitrile;
(S)-2-Amino-1-(3,3-difluoropyrrolidin-1-yl)-3-(2-fluoro-4-{3-[1-(3-isopropyl-[1,2,4]oxadiazol-5-yl)piperidin-4-yl]propoxy}phenyl)propan-1-one;
(S)-2-Amino-3-(2-fluoro-4-{3-[1-(3-isopropyl-[1,2,4]oxadiazol-5-yl)piperidin-4-yl]propoxy}phenyl)-1-((S) -3-fluoropyrrolidin-1-yl)propan-1-one;
(2S,3S)-2-Amino-1-(3,3-difluoropyrrolidin-1-yl)-3-(2-fluoro-4-{(R)-3-[1-(3-isopropyl-[1,2,4]oxadiazol-5-yl)piperidin-4-yl]butoxy}phenyl)butan-1-one;
(S)-1-[(2S,3S)-2-Amino-3-(2-fluoro-4-{(R)-3-[1-(3-isopropyl-[1,2,4]oxadiazol-5-yl)piperidin-4-yl]butoxy}phenyl)butyryl]pyrrolidine-2-carbonitrile;
4-(3-{4-[(1S,2S)-2-Amino-3-(3,3-difluoropyrrolidin-1-yl)-1-methyl-3-oxopropyl]-3-fluorophenoxy}propyl)piperidine-1-carboxylic acid isopropyl ester;
(S)-1-[(S)-2-Amino-2-(4-{(R)-3-[1-(3-isopropyl-[1,2,4]oxadiazol-5-yl)piperidin-4-yl]butoxy}phenyl)acetyl]pyrrolidine-2-carbonitrile;
(S)-2-Amino-1-(3,3-difluoropyrrolidin-1-yl)-2-(4-{(R)-3-[1-(3-isopropyl-[1,2,4]oxadiazol-5-yl)piperidin-4-yl]butoxy}phenyl)ethanone;
(S)-1-[(S)-2-Amino-3-(4-{3-[1-(3-isopropyl-[1,2,4]oxadiazol-5-yl)piperidin-4-yl]-propoxy}-2-methylphenyl)propionyl]pyrrolidine-2-carbonitrile;
(S)-2-Amino-1-(3,3-difluoropyrrolidin-1-yl)-2-(2-fluoro-4-{(R)-3-[1-(3-isopropyl-[1,2,4]oxadiazol-5-yl)piperidin-4-yl]butoxy}phenyl)ethanone;
(S)-1-[(S)-2-Amino-2-(2-fluoro-4-{(R)-3-[1-(3-isopropyl-[1,2,4]oxadiazol-5-yl)-piperidin-4-yl]butoxy}phenyl)acetyl]pyrrolidine-2-carbonitrile;
(S)-1-[(S)-2-Amino-2-(4-{(R)-3-[1-(3-isopropyl-[1,2,4]oxadiazol-5-yl)piperidin-4-yl]butoxy}-2-methylphenyl)acetyl]pyrrolidine-2-carbonitrile;
(S)-2-Amino-3-(4-{(R)-4-[1-(5-chloropyrimidin-2-yl)-piperidin-4-yl]pentyl}-2-fluorophenyl)-1-(3,3-difluoropyrrolidin-1-yl)propan-1-one;
(R)-2-(2,5-Difluorophenyl)-1-(4-{(R)-3-[1-(3-isopropyl-[1,2,4]oxadiazol-5-yl)-piperidin-4-yl]butoxy}phenyl)ethylamine;
(S)-2-Amino-2-(4-{(R)-3-[1-(5-chloropyrimidin-2-yl)piperidin-4-yl]butoxy}phenyl)-1-(3,3-difluoropyrrolidin-1-yl)ethanone;
(S)-1-[(S)-2-Amino-2-(4-{(R)-3-[1-(3-ethyl-[1,2,4]oxadiazol-5-yl)piperidin-4-yl]-butoxy}phenyl)acetyl]pyrrolidine-2-carbonitrile;
(S)-1-[(S)-2-Amino-2-(2-fluoro-4-{3-[1-(3-isopropyl-[1,2,4]oxadiazol-5-yl)piperidin-4-yl]propoxy}phenyl)acetyl]pyrrolidine-2-carbonitrile;
and pharmaceutically acceptable salts thereof.

13. A pharmaceutical composition comprising a compound according to claim 1, or a pharmaceutically acceptable salt thereof; and a pharmaceutically acceptable carrier.

14. A method for the treatment of a disease or condition in which GPR119 and DPP-IV play a role comprising a step of administering to a subject in need thereof an effective amount of a compound according to claim 1, or a pharmaceutically acceptable salt thereof.

15. A method for the treatment of type II diabetes comprising a step of administering to a subject in need thereof an effective amount of a compound according to claim 1, or a pharmaceutically acceptable salt thereof.

16. A method for the treatment of obesity, metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound according to claim 1, or a pharmaceutically acceptable salt thereof.

17. A compound of any one of formulae (IV), (V), (VI), (XLIII), (XLV), (LI), (LII), (LIII) or (LIV), wherein:
R¹, R², R³, R⁷, X, Y, W, m and n are as defined in claim 1;
R⁴ is as defined in claim 1 or a protecting group;
PG is a protecting group;
Ak is C₁₋₂ alkyl; and
G is 5- or 6-membered heteroaryl:

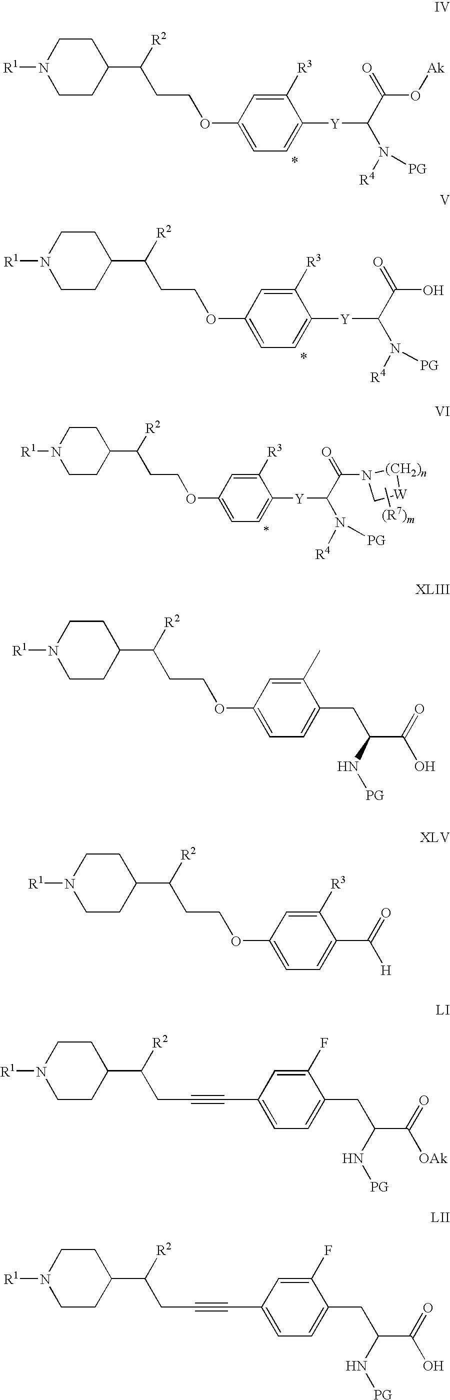

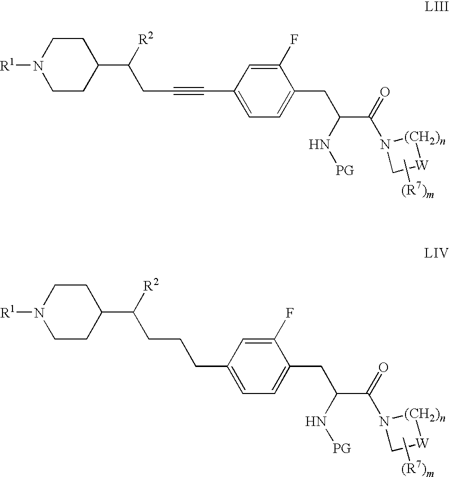

18. A pharmaceutical composition comprising a compound according to claim 3, or a pharmaceutically acceptable salt thereof; and a pharmaceutically acceptable carrier.

19. A method for the treatment of a disease or condition in which GPR119 and DPP-IV play a role comprising a step of administering to a subject in need thereof an effective amount of a compound according to claim 3, or a pharmaceutically acceptable salt thereof.

20. A method for the treatment of type II diabetes comprising a step of administering to a subject in need thereof an effective amount of a compound according to claim 3, or a pharmaceutically acceptable salt thereof.

21. A method for the treatment of obesity, metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound according to claim 3, or a pharmaceutically acceptable salt thereof.
